SEQUENCE LISTING (27 sequences)

SEQ ID NO: 1 - DNA, 288 bp, Homo sapiens
  CDS (1)...(365)
atggcccccccgcccgcgtgccggtccccgatgtcaccgccgccgccgccgctgctgctgctgctgctgagtctggcgctgctgggcgcccgggcccgcgccgagcccgccgggagtgccgtccccgcgcagagccgcccatgcgtggactgccacgccttcgagttcatgcagcgcgccctgcaggacctgcggaagacagcctgcagcctggacgcgcggacggagaccctactgctgcaggcagagcgccgtgccctgtgtgcctgctggccagcggggcactga

SEQ ID NO: 2 - protein, 95 aa, Homo sapiens
MAPPPACRSPMSPPPPPLLLLLLSLALLGARARAEPAGSAVPAQSRPCVDCHAFEFMQRALQDLRKTACSLDARTETLLLQAERRALCACWPAGH

SEQ ID NO: 3 - DNA, 285 bp, Artificial Sequence
  Degenerate nucleotide sequence
atggcnccnccnccngcntgymgnwsnccnatgwsnccnccnccnccnccnytnytnytnytnytnytnwsnytngcnytnytnggngcnmgngcnmgngcngarccngcnggnwsngcngtnccngcncarwsnmgnccntgygtngaytgycaygcnttygarttyatgcarmgngcnytncargayytnmgnaaracngcntgywsnytngaygcnmgnacngaracnytnytnytncargcngarmgnmgngcnytntgygcntgytggccngcnggncay

SEQ ID NO: 4 - DNA, 258 bp, Homo sapiens
  CDS (1)...(258)
atgtcaccgccgccgccgccgctgctgctgctgctgctgagtctggcgctgctgggcgcccgggcccgcgccgagcccgccgggagtgccgtccccgcgcagagccgcccatgcgtggactgccacgccttcgagttcatgcagcgcgccctgcaggacctgcggaagacagcctgcagcctggacgcgcggacggagaccctactgctgcaggcagagcgccgtgccctgtgtgcctgctggccagcggggcactga

SEQ ID NO: 5 - protein, 85 aa, Homo sapiens
MSPPPPPLLLLLLSLALLGARARAEPAGSAVPAQSRPCVDCHAFEFMQRALQDLRKTACSLDARTETLLLQAERRALCACWPAGH

SEQ ID NO: 6 - DNA, 255 bp, Artificial Sequence
  Degenerate nucleotide sequence
atgwsnccnccnccnccnccnytnytnytnytnytnytnwsnytngcnytnytnggngcnmgngcnmgngcngarccngcnggnwsngcngtnccngcncarwsnmgnccntgygtngaytgycaygcnttygarttyatgcarmgngcnytncargayytnmgnaaracngcntgywsnytngaygcnmgnacngaracnytnytnytncargcngarmgnmgngcnytntgygcntgytggccngcnggncay

SEQ ID NO: 7 - DNA, 403 bp, Homo sapiens
  CDS (108)...(365)
  variation (127)...(127): nucleic acid can be C or T
ggaacgtctgccggtgtccccgcgctgctggtcccggggtccctgaaccgcgggcggccccgctccctctgctggccatggcccccccgcccgcgtgccggtccccgatgtcaccgccgccgccgccgctgctgctgctgctgctgagtctggcgctgctgggcgcccgggcccgcgccgagcccgccgggagtgccgtccccgcgcagagccgcccatgcgtggactgccacgccttcgagttcatgcagcgcgccctgcaggacctgcggaagacagcctgcagcctggacgcgcggacggagaccctactgctgcaggcagagcgccgtgccctgtgtgcctgctggccagcggggcactgaggaccacgctgctccgtgtgaataaatgcccagtggca

SEQ ID NO: 8 - protein, 85 aa, Homo sapiens; identical to SEQ ID NO: 5
  VARIANT (17)...(17): PRO or LEU

SEQ ID NO: 9 - DNA, 18 bp, Artificial Sequence
  Oligonucleotide primer, ZC 39486
actgccacgccttcgagt

SEQ ID NO: 10 - DNA, 19 bp, Artificial Sequence
  Oligonucleotide primer, ZC 39487
agcagtagggtctccgtcc

SEQ ID NO: 11 - DNA, 23 bp, Artificial Sequence
  Oligonucleotide primer, ZC 10565
tttgcagaaaaggttgcaaatgc

SEQ ID NO: 12 - DNA, 20 bp, Artificial Sequence
  Oligonucleotide primer, ZC 10651
agcttttctgcagcagctct

SEQ ID NO: 13 - DNA, 49 bp, Artificial Sequence
  Polynucleotide encoding C terminal His tag
ggctcaggatctggtggcggccatcaccaccatcatcactaaatctaga

SEQ ID NO: 14 - DNA, 39 bp, Artificial Sequence
  Oligonucleotide primer, ZC48711
gaagaacgtctctcatgtcaccgccgccgccgccgctgc

SEQ ID NO: 15 - DNA, 39 bp, Artificial Sequence
  Oligonucleotide primer, ZC48196
gaagaacgtctctagccgtgccccgctggccagcaggca

SEQ ID NO: 16 - DNA, 21 bp, Artificial Sequence
  Oligonucleotide primer, ZC5020
cactggagtggcaacttccag

SEQ ID NO: 17 - DNA, 25 bp, Artificial Sequence
  Oligonucleotide primer, ZC14063
caccagacataatagctgacagact

SEQ ID NO: 18 - protein, 61 aa, Homo sapiens
  MOD_RES (5)...(5): Ortho glycolylation
EPAGSAVPAQSRPCVDCHAFEFMQRALQDLRKTACSLDARTETLLLQAERRALCACWPAGH

SEQ ID NO: 19 - protein, 27 aa, Artificial Sequence
  Synthetic C terminal polypeptide fragment of Zsig98
CSLDARTETLLLQAERRALCACWPAGH

SEQ ID NO: 20 - protein, 34 aa, Artificial Sequence
  Synthetic N terminal polypeptide fragment of Zsig98
EPAGSAVPAQSRPCVDCHAFEFMQRALQDLRKTA

SEQ ID NO: 21 - protein, 61 aa, Artificial Sequence; identical to SEQ ID NO: 18
  Synthetic ligated mature Zsig98 polypeptide

SEQ ID NO: 22 - DNA, 258 bp, Homo sapiens; identical to SEQ ID NO: 4

SEQ ID NO: 23 - protein, 61 aa, Artificial Sequence
  Consensus mature Zsig98 polypeptide
XXXGSAVPAQSRPCVDCHAFEFMQRALQDLRKTACSLDARTETLLLQAERRALCACWPAGH

SEQ ID NO: 24 - protein, 122 aa, Mus musculus
MRARAGRGRSSRVARAACSRFLEPRAAWAPSAMAPALRSLLSPRTLLLLLLSLALLGARAEPATGSAVPAQSRPCVDCHAFEFMQRALQDLRKTAYSLDARTETLLLQAERRALCACWPAGR

SEQ ID NO: 25 - protein, 138 aa, Rat
MTTTSGAPGWGPLGRMRARAGRGPSFPSRPRCCSRFCGPRAAWAPSAMAPAPRSLLSPPILLLLLLLSLALLGARAEPAAGSAVPAQSRPCVDCHAFEFMQRALQDLRKTAYSLDARTETLLLQAERRALCACWPAGR

SEQ ID NO: 26 - protein, 85 aa, Zebra fish
MRPSGALAAAAVMLMLLVCCQAEDSGSVIPAESRPCVDCHAFEFMQRALQDLKKTAFNLDSRTENLVLRAEKRALCDCMPASSLR

SEQ ID NO: 27 - protein, 95 aa, Chicken
TMLVPCLRRRLVLTVPLVFVAALLLMEPARSDQEAGTAIPAESRPCVDCHAFEFMQRALQDLKKTAYNLDTRTETLLLQVEKRNLCDCVTANLLN

(19) United States
(12) Patent Application Publication
(10) Pub. No.: US 2006/0258586 A1
(43) Pub. Date: Nov. 16, 2006

(54) Compositions and uses of secreted polypeptide, Zsig98

(76) Inventors: Paul O. Sheppard, Granite Falls, WA (US); Stephen R. Jaspers, Edmonds, WA (US); Michael R. Stamm, Everett, WA (US); Anitra Wolf, Seattle, WA (US); Jacob J. Kennedy, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 11/418,803

(22) Filed: May 5, 2006

Related U.S. Application Data
(60) Provisional application No. 60/678,882, filed on May 6, 2005.
(60) Provisional application No. 60/758,889, filed on Jan. 13, 2006.
(60) Provisional application No. 60/784,271, filed on Mar. 21, 2006.

(51) Int. Cl. C07K 14/705; A61K 38/17

(52) U.S. Cl. 514/12; 530/324

(57) ABSTRACT

The present invention provides methods of using Zsig98 polypeptides for treating intestinal motility disorders and improving gastrointestinal function, nutritient absorption, metabolism, and diabetes with Zsig98 polypeptides. The invention also provides methods for producing Zsig98 polynucleotides, polypeptides and antibodies.

### CROSS REFERENCE TO RELATED APPLICATIONS

This application claims the benefit of U.S. Provisional Application Ser. No. 60/678,882, filed May 6, 2005, U.S. Provisional Application Ser. No. 60/758,889, filed Jan. 13, 2006, and U.S. Provisional Application Ser. No. 60/784,271, filed Mar. 21, 2006, all of which are herein incorporated by reference.

### BACKGROUND OF THE INVENTION

Many of the regulatory peptides that are important in maintaining nutritional homeostasis are found in the gastrointestinal environment. These peptides may be synthesized in the digestive system and act locally, but can also be identified in the brain as well. In addition, the reverse is also found, i.e., peptides are synthesized in the brain, but found to regulate cells in the gastrointestinal tract. This phenomenon has been called the “brain-gut axis” and is important for signaling satiety, regulating body temperature and other physiological processes that require feedback between the brain and gut.

The gut peptide hormones include gastrin, cholecystokinin (CCK), secretin, gastric inhibitory peptide (GIP), vasoactive intestinal polypeptide (VIP), motilin, somatostatin, pancreatic peptide (PP), substance P neuropeptide Y (NPY), and ghrelin, and use several different mechanisms of action. For example, gastrin, motilin and CCK function as endocrine- and neurocrine-type hormones. Others, such as gastrin and GIP, are thought to act exclusively in an endocrine fashion. Other modes of action include a combination of endocrine, neurocrine and paracrine action (somatostatin); exclusively neurocrine action (NPY); and a combination of neurocrine and paracrine actions (VIP and Substance P). Most of the gut hormone actions are mediated by membrane-bound receptors and activate second messenger systems. For a review of gut peptides see, Mulvihill et al., in Basic and Clinical Endocrinology, pp. 551-570, 4th edition Greenspan F. S. and Baxter, J. D. editors., Appleton & Lange: Norwalk, Conn., 1994. In addition, see Konturek, S. J., et al., J. Physiol. and Pharmacol., 54:3: 294-317, (2003).

Many of these gut peptides are synthesized as inactive precursor molecules that require multiple peptide cleavages to be activated. The family known as the “glucagon-secretin” family which includes VIP, gastrin, secretin, motilin, glucagon and galanin exemplifies peptides regulated by multiple cleavages and post-translational modifications.

Optimal gastrointestinal function includes mixing and forward propulsion of contents in the stomach and intestine. Gastric emptying is frequently abnormal in patients with critical illness or who are recovering from surgery. Recovery of gastrointestinal function and resumption of oral intake are important determinants in recovery from an event that compromises gastrointestinal function. Several events can lead to dysfunction in the gastrointestinal system, including, for example, ileus (post-operative and paralytic), chronic constipation, gastroparesis (including diabetic gastroparesis), intestinal pseudo-obstruction, dyspepsia, gastroesophageal reflux, and emesis.

Diseases and disorders of impaired or compromised gastrointestinal function include ileus and gastroparesis. Post-operative ileus (POI) is a condition of reduced intestinal tract motility, including delayed gastric emptying, that occurs as a result of disrupted muscle tone following surgery. It is especially problematic following abdominal surgery. The problem may arise from the surgery itself, from the residual effects of anesthetic agents, and particularly, from pain-relieving narcotic and opiate drugs used during and after surgery. Post-operative ileus can be categorized as “uncomplicated”, lasting two to three days after surgery, or as “paralytic”, lasting more than three days after surgery. Thus, patients undergoing abdominal surgery who have a delay in recovery of gastrointestinal function have prolonged hospital stays, which can lead to increased medical costs and potentially to other complications. An estimated 750 million to one billion dollars is spent each year in increased hospitalization due to post-operative ileus. Currently there are no drugs that have been approved for treatment of this disease.

In addition to the need for a better therapeutic for post-operative ileus, there is a need for a better therapeutic for diabetic gastroparesis. Diabetic gastroparesis is paralysis of the stomach brought about by a motor abnormality in the stomach, as a complication of both type I and type II diabetes. Diabetic gastroparesis is characterized by delayed gastric emptying, post-prandial distention, nausea and vomiting. In diabetes, it is thought to be due to a neuropathy, though it is also associated with loss of interstitial cells of Cajal (ICC), which are the “pacemaker cells” of the gut.

In the U.S. alone, there are at least 16 million individuals with diabetes, affecting approximately 7% of the population. The prevalence is continuing to increase and is growing worldwide. Since up to two-thirds of individuals with diabetes suffer from some degree of gastroparesis, this problem is significant. Episodes are often acute, though long-term treatment is often required. Moreover, symptoms associated with diabetic gastroparesis, such as delayed gastric emptying, and emesis can cause water and electrolyte imbalances, poor glycemic control, and ensuing complications. If severe enough, it may require hospitalization for control of diabetes, and treatment with intravenous fluids and nutrition.

The often-acute nature of the episodes provides an opportunity to treat with a prokinetic. Currently there are very few drugs that can effectively treat diabetic gastroparesis, and those that are available have side effects and/or cannot be taken with other medications. Oral drugs may not be tolerated during severe episodes, and thus, would require intravenous administration of a prokinetic. In the United States, only two agents, erythromycin and metoclopramide, are available to treat gastroparesis.

Increased insulin sensitivity, and beta cell mass in Diabetics is a critical need for many diabetic patients. Novel growth factors that stimulate beta cell function and/or proliferation represent potential therapeutic molecules. Blood glucose homeostasis is controlled by the endocrine cells of the pancreas, located in the islets of Langerhans. Beta cells are the most numerous islet cells. The islet cells monitor the concentration of glucose in the blood, secreting hormones having opposite effects. After a meal the blood glucose concentration increases causing the beta cells to secrete the hormone insulin, thereby reducing blood glucose. Insulin stimulates the uptake of glucose by cells of the body as well as stimulates the conversion of glucose to glycogen in the liver. If the glucose level falls too far, islet alpha cells secrete the hormone glucagons. Glucagon stimulates the breakdown of glycogen to glucose in the liver, increasing blood glucose between meals. Blood glucose levels can be best controlled by minor changes in insulin production and secretion. In addition, blood glucose levels are controlled by the pancreatic beta cells, which can release insulin on demand and which have the capacity to secrete large amounts of insulin, for example, in response to a heavy meal. The need for the beta-cell mass to be closely regulated by glucose and hormonal effects on beta-cell replication, size, apoptotic elimination and, under certain conditions, neogenesis from progenitor cells is extremely important. Changes in body mass, pregnancy, insulin sensitivity of peripheral tissues, or tissue injury, if not successfully adapted may lead to the development of chronically elevated blood glucose, or diabetes. The prevalence of diabetes on a global level has stimulated efforts to develop new therapeutic strategies like beta-cell replacement or regenerative medicine. Existing therapies with exogenous insulin or hypoglycemic agents for type 1 and type 2 diabetes are unsatisfactory, and do not offer a cure and are mostly insufficient for preventing the secondary complications associated with diabetes. See Bouwens, L. et al., Physiol Rev 85:1255-1270, 2005.

Thus, a need still exists for therapeutic approaches to treatment of gastric function disorders and nutrient dysfunction.

### BRIEF SUMMARY OF THE INVENTION

The present invention provides proteins useful for the treatment in recovery of gastronintestinal function, gastric emptying and nutrient absorption. Other uses of Zsig98 polypeptides are described in more detail below.

Within one aspect the invention provides an isolated polypeptide comprises or consists of the amino acid sequence as shown in SEQ ID NO: 2 from amino acid 35 to amino acid 95.

Within another aspect the invention provides an isolated polypeptide comprises or consists of the amino acid sequence as shown in SEQ ID NO: 18 from amino acid 1 to amino acid 61.

Within another aspect the invention provides a polynucleotide encoding the polypeptide comprising or consisting of the amino acid sequence as shown in SEQ ID NO: 2 from amino acid 35 to amino acid 95.

Within another aspect the invention provides a polynucleotide encoding the polypeptide comprising or consisting of the amino acid sequence as shown in SEQ ID NO: 18 from amino acid 1 to amino acid 61.

Within another aspect the invention provides a composition for improving nutrient absorption wherein the composition comprises the polypeptide comprising or consisting of the amino acid sequence from amino acid 35 to amino acid 95 of SEQ ID NO: 2 and wherein the polypeptide is combined with a nutrient mixture. In an embodiment, the nutrient mixture is milk or a milk substitute.

Within another aspect the invention provides a composition for improving nutrient absorption wherein the composition comprises the polypeptide comprising or consisting of the amino acid sequence from amino acid 1 to amino acid 61 of SEQ ID NO: 18 and wherein the polypeptide is combined with a nutrient mixture. In an embodiment, the nutrient mixture is milk or a milk substitute.

Within another aspect the invention provides a method of modulating secretion of hormones in vitro or in vivo comprising administering the polypeptide comprising or consisting of residues 35 to 95 of SEQ ID NO:2 or the polypeptide comprising or consisting of residues 1 to 61 of SEQ ID NO: 18, wherein the polypeptide forms a peptide-receptor complex with a receptor, and wherein the formation of the peptide-receptor complex modulates the secretion of hormones in the cells.

Within another aspect the invention provides a method of improving neural development and/or utilization in a mammal comprising administering the peptide comprising or consisting of residues 35 to 95 of SEQ ID NO:2 or the polypeptide comprising or consisting of residues 1 to 61 of SEQ ID NO: 18, wherein neural development and/or utilization in the mammal is improved.

Within another aspect the invention provides a method of inducing or inhibiting contractility in gastrointestinal cells comprising administering the peptide comprising or consisting of residues 35 to 95 of SEQ ID NO:2 or the polypeptide comprising or consisting of residues 1 to 61 of SEQ ID NO: 18, or an antagonist thereof, wherein in the gastrointestinal cells contract.

Within another aspect the invention provides a method of improving nutrient uptake in gastrointestinal cells comprising administering the peptide comprising or consisting of residues 35 to 95 of SEQ ID NO:2 or the polypeptide comprising or consisting of residues 1 to 61 of SEQ ID NO: 18, wherein nutrient uptake is measured in the gastrointestinal cells.

Within another aspect the invention provides a method of inducing growth hormone secretion in pituitary cells comprising administering the polypeptide comprising or consisting of residues 35 to 95 of SEQ ID NO:2 or the polypeptide comprising or consisting of residues 1 to 61 of SEQ ID NO: 18, wherein growth hormone is secreted.

Within another aspect the invention provides a method of inducing secretion of digestive enzymes in gastrointestinal cells comprising administering the polypeptide comprising or consisting of residues 35 to 95 of SEQ ID NO:2 or the polypeptide comprising or consisting of residues 1 to 61 of SEQ ID NO: 18, wherein secretion of digestive enzymes in the gastrointestinal cells is observed.

Within another aspect the invention provides a method of inducing the secretion of digestive hormones in gastrointestinal cells comprising administering the polypeptide comprising or consisting of residues 35 to 95 of SEQ ID NO:2 or the polypeptide comprising or consisting of residues 1 to 61 of SEQ ID NO: 18, wherein the administration results in secretion of digestive hormones in the gastrointestinal cells.

Within another aspect the invention provides a method of inducing secretion of enzymes and hormones secreted from endocrine, exocrine, or gastrointestinal cells comprising administering the polypeptide comprising or consisting of residues 35 to 95 of SEQ ID NO:2 or the polypeptide comprising or consisting of residues 1 to 61 of SEQ ID NO: 18, wherein the administration results in secretion of enzymes and hormones. Within an embodiment, the cells are alpha, beta, or acinar cells.

Within another aspect the invention provides a method of inducing secretion of enzymes in pancreas cells comprising administering the the polypeptide comprising or consisting of residues 35 to 95 of SEQ ID NO:2 or the polypeptide comprising or consisting of residues 1 to 61 of SEQ ID NO: 18, wherein the administration results in secretion of enzymes in the pancreas cells.

Within another aspect the invention provides a method of inducing the secretion of hormones in pancreas cells comprising administering the polypeptide comprising or consisting of residues 35 to 95 of SEQ ID NO:2 or the polypeptide comprising or consisting of residues 1 to 61 of SEQ ID NO: 18 wherein the administration results in secretion of hormones in the pancreas cells.

Within another aspect the invention provides a method of inhibiting or reducing gastric reflux in gastrointestinal tissue comprising administering the polypeptide comprising or consisting of residues 35 to 95 of SEQ ID NO:2 or the polypeptide comprising or consisting of residues 1 to 61 of SEQ ID NO: 18 wherein the administration results a inhibition or in reduction or gastric reflux in the gastrointestinal tissue.

Within another aspect the invention provides a method of inducing the secretion of insulin-like growth factor-I in cells comprising administering the polypeptide comprising or consisting of residues 35 to 95 of SEQ ID NO:2 or the polypeptide comprising or consisting of residues 1 to 61 of SEQ ID NO: 18, wherein results in the secretion of insulin-like growth factor-I in the cells.

Within another aspect the invention provides a method of treating a mammal having a metabolic disorder, wherein the metabolic disorder is selected from the group consisting of: satiety regulation; glucose absorption; glucose metabolism; and neuropathy-associated gastrointestinal disorders and wherein the method comprises, administering the the polypeptide comprising or consisting of residues 35 to 95 of SEQ ID NO:2 or the polypeptide comprising or consisting of residues 1 to 61 of SEQ ID NO: 18, and wherein metabolic state of mammal having the metabolic disorder is improved.

Within another aspect the invention provides a method of stimulating glucose-induced insulin release in a mammal comprising administering the polypeptide comprising or consisting of residues 35 to 95 of SEQ ID NO:2 or the polypeptide comprising or consisting of residues 1 to 61 of SEQ ID NO: 18, wherein the administration induces insulin release in the mammal.

Within another aspect the invention provides an isolated polypeptide comrprising the amino acid sequence as shown in SEQ ID NO:23. Within an embodiment, the polypeptide consists of the amino acid sequence as shown in SEQ ID NO: 23. Within another embodiment, the polypeptide consists of the amino acid sequence as shown in SEQ ID NO: 18.

Within another aspect the invention provides a composition for improving nutrient absorption wherein the composition comprises the polypeptide comprising or consisting of residues 1 to 61 of SEQ ID NO: 18 and wherein the polypeptide is combined with a nutrient mixture. In an embodiment, the nutrient mixture is milk or a milk substitute.

Within another aspect the invention provides a method of inducing proliferation of cells in the pancreas comprising subjecting the cells to a polypeptide comprising or consisting of residues 35 to 95 of SEQ ID NO:2 or the polypeptide comprising or consisting of residues 1 to 61 of SEQ ID NO: 18. Within an embodiment the cells are endocrine cells. Within another embodiment, the cells are selected from: a) acinar cells; b) alpha cells; and c) beta cells.

Within another aspect the invention provides a method of increasing cell mass of cells of the pancreas comprising exposing the pancreas cells to a polypeptide comprising or consisting of residues 35 to 95 of SEQ ID NO:2 or the polypeptide comprising or consisting of residues 1 to 61 of SEQ ID NO: 18. Within an embodiment the cells are endocrine cells. Within another embodiment the cells are selected from: a) acinar cells; b) alpha cells; and c) beta cells.

Within another embodiment the invention provides a method of producing Zsig98 dimers. Within one embodiment the dimers are produced form host cells. Within a further embodiment the Zsig98 dimers are produced by chemical synthesis.

Within another aspect the invention provides an antibody that specifically binds to Zsig98 polypeptides. Within an embodiment the antibody specifically binds to monomers of the Zsig98 polypeptide. Within another embodiment, the antibody specifically binds to dimers of the Zsig98 polypeptides. Within another embodiment, the antibody specifically binds to multimers of the Zsig98 polypeptides.

Within another aspect the invention provides antibodies that specifically bind to monomers, dimers, or multimers of the Zsig98 polypeptides and such binding of the antibody modulates the secretion of enzymes and hormones from endocrine, exocrine, or gastrointestinal cells. Such antibodies are useful in treating, preventing, reducing, or limiting endocrine or exocrine dysfunction.

### DESCRIPTION OF THE INVENTION

### 1. Overview

The present invention is directed to novel compositions and uses of a secreted polypeptide, Zsig98. As discussed herein, Zsig98, as well as variants and fragments thereof, can be used to regulate gastrointestinal function, gastric emptying and nutrient absorption.

The present invention provides methods of using human Zsig98 polypeptides and nucleic acid molecules that encode human Zsig98 polypeptides. An illustrative nucleic acid molecule containing a sequence that encodes the Zsig98 polypeptide has the nucleotide sequence of SEQ ID NO:1 and the encoded polypeptide has the amino acid sequence as shown in SEQ ID NO:2. Thus, the Zsig98 nucleotide sequence described herein encodes a polypeptide 95 amino acids as shown in SEQ ID NO: 2. The putative signal sequences of Zsig98 polypeptide reside at amino acid residues 1 to 36, 1 to 28, 1 to 29, 1 to 30, 1 to 31, 1 to 33, and 1 to 34 SEQ ID NO:2. The mature form of the polypeptide comprises the amino acid sequence from amino acid 44 to 95 as shown in SEQ ID NO:2. In another embodiment, the mature form of the polypeptide comprises the amino acid sequence from amino acid 35 to 95 as shown in SEQ ID NO:2. The polynucletotide encoding the mature polypeptide starts at postion 103 of SEQ ID NO: 1 and ends at position 288 of SEQ ID NO: 1, or starts at position 128 of SEQ ID NO: 1 and ends at position 288 of SEQ ID NO: 1.

Variant forms of the sequence as shown in SEQ ID NO:2 are included in the invention described herein. A variant form has the amino acid sequence as shown in SEQ ID NO:5, and is coded for by the polynucleotide sequence as shown in SEQ ID NO: 4. The putative signal sequences of Zsig98 polypeptide reside at amino acid residue positions similar to those of SEQ ID NO:2.

As described below, the present invention provides isolated polypeptides comprising an amino acid sequence that is at least 70%, at least 75%, at least 80%, at least 85%, at least 90%, or at least 95% identical to amino acid residues 25 to 85 of SEQ ID NO:2 or to amino acid residues 35 to 95 of SEQ ID NO: 2. Certain of such isolated polypeptides can specifically bind with an antibody that specifically binds with a polypeptide consisting of the amino acid sequence of SEQ ID NOs:2 or 5. Particular polypeptides can increase or decrease gastric contractility, gastric emptying and/or intestinal transit. An illustrative polypeptide is a polypeptide that comprises the amino acid sequence of SEQ ID NO:2.

The present invention also provides polypeptides comprising an amino acid sequence selected from the group consisting of: amino acid residues 25 to 95 of SEQ ID NO:2; and/or amino acid residues 25 to 85 of SEQ ID NO: 5.

The present invention further provides antibodies and antibody fragments that specifically bind with such polypeptides. Exemplary antibodies include polyclonal antibodies, murine monoclonal antibodies, humanized antibodies derived from murine monoclonal antibodies, and human monoclonal antibodies. Illustrative antibody fragments include F(ab′)₂, F(ab)₂, Fab′, Fab, Fv, scFv, and minimal recognition units. The present invention also includes anti-idiotype antibodies that specifically bind with such antibodies or antibody fragments. The present invention further includes compositions comprising a carrier and a peptide, polypeptide, antibody, or anti-idiotype antibody described herein.

The present invention also provides isolated nucleic acid molecules that encode a Zsig98 polypeptide, wherein the nucleic acid molecule is selected from the group consisting of (a) a nucleic acid molecule comprising the nucleotide sequence of SEQ ID NO:3, (b) a nucleic acid molecule encoding the amino acid sequence of SEQ ID NO:2, (c) a nucleic acid molecule that remains hybridized following stringent wash conditions to a nucleic acid molecule consisting of the nucleotide sequence of nucleotides 66 to 161 of SEQ ID NO: 1, or to the complement thereof.

Illustrative nucleic acid molecules include those in which any difference between the amino acid sequence encoded by the nucleic acid molecule and the corresponding amino acid sequence of SEQ ID NO:2 is due to a conservative amino acid substitution. The present invention further contemplates isolated nucleic acid molecules that comprise a nucleotide sequence of nucleotides 132 to 389 of SEQ ID NO:1, nucleotides 147 to 389 of SEQ ID NO:1.

The present invention also includes vectors and expression vectors comprising such nucleic acid molecules. Such expression vectors may comprise a transcription promoter, and a transcription terminator, wherein the promoter is operably linked with the nucleic acid molecule, and wherein the nucleic acid molecule is operably linked with the transcription terminator. The present invention further includes recombinant host cells comprising these vectors and expression vectors. Illustrative host cells include bacterial, yeast, avian, fungal, insect, mammalian, and plant cells. Recombinant host cells comprising such expression vectors can be used to prepare Zsig98 polypeptides by culturing such recombinant host cells that comprise the expression vector and that produce the Zsig98 protein, and, optionally, isolating the Zsig98 protein from the cultured recombinant host cells. The present invention further includes products made by such processes.

In addition, the present invention provides pharmaceutical compositions comprising a pharmaceutically acceptable carrier and at least one of such an expression vector or recombinant virus comprising such expression vectors.

The present invention also contemplates methods for detecting the presence of Zsig98 RNA in a biological sample, comprising the steps of (a) contacting a Zsig98 nucleic acid probe under hybridizing conditions with either (i) test RNA molecules isolated from the biological sample, or (ii) nucleic acid molecules synthesized from the isolated RNA molecules, wherein the probe has a nucleotide sequence comprising a portion of the nucleotide sequence of SEQ ID NO: 1, or its complement, and (b) detecting the formation of hybrids of the nucleic acid probe and either the test RNA molecules or the synthesized nucleic acid molecules, wherein the presence of the hybrids indicates the presence of Zsig98 RNA in the biological sample.

The present invention further provides methods for detecting the presence of Zsig98 polypeptide in a biological sample, comprising the steps of: (a) contacting the biological sample with an antibody or an antibody fragment that specifically binds with a polypeptide either consisting of the amino acid sequence of SEQ ID NO:2 or consisting of the amino acid sequence of SEQ ID NO:5, wherein the contacting is performed under conditions that allow the binding of the antibody or antibody fragment to the biological sample, and (b) detecting any of the bound antibody or bound antibody fragment. Such an antibody or antibody fragment may further comprise a detectable label selected from the group consisting of radioisotope, fluorescent label, chemiluminescent label, enzyme label, bioluminescent label, and colloidal gold.

Illustrative biological samples include human tissue, such as an autopsy sample, a biopsy sample, body fluids and digestive components, and the like.

The present invention also provides kits for performing these detection methods. For example, a kit for detection of Zsig98 gene expression may comprise a container that comprises a nucleic acid molecule, wherein the nucleic acid molecule is selected from the group consisting of (a) a nucleic acid molecule comprising the nucleotide sequence of nucleotides 66 to 161 of SEQ ID NO:1, (b) a nucleic acid molecule comprising the nucleotide sequence of nucleotides 288 to 389 of SEQ ID NO:1, (c) a nucleic acid molecule comprising the complement of the nucleotide sequence of nucleic acid molecules (a) or (b), (d) a nucleic acid molecule that is a fragment of (a) consisting of at least eight nucleotides, (e) a nucleic acid molecule that is a fragment of (b) consisting of at least eight nucleotides, (f) a nucleic acid molecule that is a fragment of (c) consisting of at least eight nucleotides, and (g) a nucleic acid molecule that is a fragment of or consists of the nucleic acid sequence as shown in SEQ ID NO: 1, 3, 4, or 7. Such kits may also comprise a second container that comprises one or more reagents capable of indicating the presence of the nucleic acid molecule.

On the other hand, a kit for detection of Zsig98 protein may comprise a container that comprises an antibody, or an antibody fragment, that specifically binds with a polypeptide consisting of the amino acid sequence of SEQ ID NO:2 or consisting of the amino acid sequence of SEQ ID NO:5.

The present invention also contemplates anti-idiotype antibodies, or anti-idiotype antibody fragments, that specifically bind an antibody or antibody fragment that specifically binds a polypeptide consisting of the amino acid sequence of SEQ ID NO:2 or the amino acid sequence of SEQ ID NO:5.

The present invention further provides variant Zsig98 polypeptides, which comprise an amino acid sequence that shares an identity with the amino acid sequence of SEQ ID NO:2 selected from the group consisting of at least 70% identity, at least 80% identity, at least 90% identity, at least 95% identity, or greater than 95% identity, and wherein any difference between the amino acid sequence of the variant polypeptide and the amino acid sequence of SEQ ID NO:2 is due to one or more conservative amino acid substitutions.

The present invention also provides fusion proteins comprising a Zsig98 polypeptide moiety. Such fusion proteins can further comprise an immunoglobulin moiety. A suitable immunoglobulin moiety is an immunoglobulin heavy chain constant region, such as a human F_{C} fragment. The present invention also includes isolated nucleic acid molecules that encode such fusion proteins.

The present invention further provides a method of treating defective ileal contractility disease in a mammalian subject in need of such treatment, comprising: administering to the mammalian subject a Zsig98 polypeptide, wherein the Zsig98 polypeptide comprises the amino acid sequence of amino acid residues 44 to 95 of SEQ ID NO:2, or 44 to 85 of SEQ ID NO: 5. In one embodiment, the disease is diabetes mellitus. In another method, the disease is post-operative ileus. In another embodiment, the disease is sepsis-related gastrointestinal stasis or ileus. In another embodiment, the polypeptide comprises the amino acid sequence from amino acid 35 to 95 as shown in SEQ ID NO:2.

The present invention further provides a method of treating defective ileal contractility disease in a mammalian subject in need of such treatment, comprising: administering to the mammalian subject a Zsig98 polypeptide, wherein the Zsig98 polypeptide comprises the amino acid sequence of amino acid residues 44 to 95 of SEQ ID NO:2, or the amino acid sequence of amino acid residues 35 to 95 of SEQ ID NO: 2, or the amino acid sequence of amino acid residues 44 to 85 of SEQ ID NO:5. In one embodiment, the disease is diabetes mellitus. In another embodiment, the disease is post-operative ileus. In another embodiment, the disease is sepsis-related gastrointestinal stasis or ileus.

The invention further provides a method of modulating gastrointestinal contractility in a mammal in need thereof comprising administering to the mammal a polypeptide, wherein the polypeptide comprises the amino acid sequence of amino acid residues 44 to 95 of SEQ ID NO:2, or the amino acid sequence of amino acid residues 35 to 95 of SEQ ID NO: 2. In an embodiment, the the modulation is inhibition. In another embodiment, the polypeptide is administered in one or more administrations. In a further embodiment, one or more of the administrations of the polypeptide stimulates gastrointestinal contractility, and wherein one or more of the administrations of the polypeptide inhibits gastrointestinal contractility. In another embodiment, the one or more administrations that stimulate gastrointestinal contractility are administered before the one or more administrations that inhibit gastrointestinal contractility. In another embodiment, the the one or more administrations that inhibit gastrointestinal contractility are administered before the one or more administrations that stimulate gastrointestinal contractility. In a further embodiment, therapeutic control of gastric contractility is achieved. In another embodiment, the polypeptide is administered continually for a period of time.

The invention also provides a method of stimulating gastrointestinal contractility comprising administering to a mammal in need thereof a polypeptide comprising the amino acid sequence of amino acid residues 44 to 95 of SEQ ID NO:2, or the amino acid sequence of amino acid residues 35 to 95 of SEQ ID NO: 2, or the amino acid sequence of amino acid residues 44 to 85 of SEQ ID NO:5.

The invention also provides a method of modulating gastric emptying in a mammal in need thereof comprising administering to the mammal a polypeptide, wherein the polypeptide comprises the amino acid sequence of amino acid residues 44 to 95 of SEQ ID NO:2 or the amino acid sequence of amino acid residues 35 to 95 of SEQ ID NO: 2. In an embodiment, the modulation is inhibition. In another embodiment, the polypeptide is administered in one or more administration. In another embodiment, one or more of the administrations of the polypeptide stimulates gastric emptying, and wherein one or more of the administrations of the polypeptide inhibits gastric emptying. In a further embodiment, the one or more administrations that stimulate gastric emptying are administered before the one or more administrations that inhibit gastric emptying. In another further embodiment, the one or more administrations that inhibit gastric emptying are administered before the one or more administrations that stimulate gastric emptying. In another embodiment, therapeutic control of gastric emptying is achieved. In another embodiment the polypeptide is administered continually for a period of time.

The invention also provides a method of modulating intestinal transit in a mammal in need thereof comprising administering to the mammal a polypeptide, wherein the polypeptide comprises the amino acid sequence of amino acid residues 44 to 95 of SEQ ID NO:2 or the amino acid sequence of amino acid residues 35 to 95 of SEQ ID NO: 2. In an embodiment, the modulation is inhibition. In another embodiment, the polypeptide is administered in one or more administration. In a further embodiment, one or more of the administrations of the polypeptide stimulates intestinal transit, and wherein one or more of the administrations of the polypeptide are effective in inhibiting intestinal transit. In a further embodiment, the one or more administrations that stimulate intestinal transit are administered before the one or more administrations that inhibit intestinal transit. In another further embodiment, the one or more administrations that inhibit intestinal transit are administered before the one or more administrations that stimulate intestinal transit. In another embodiment, therapeutic control of intestinal transit is achieved. In another embodiment, the polypeptide is administered continually for a period of time.

The invention also provides a method of treating gastroparesis in a mammal in thereof comprising, administering to the mammal a polypeptide, wherein the polypeptide comprises the amino acid sequence of amino acid residues 44 to 95 of SEQ ID NO:2, or the amino acid sequence of amino acid residues 35 to 95 of SEQ ID NO: 2, and wherein gastrointestinal contractility, gastric emptying, or intestinal transit is improved. In an embodiment, the gastroparesis is related to surgery. In another embodiment, the polypeptide is administered to the mammal before or after the surgery. In another embodiment, the polypeptide is administered to the mammal before or after the mammal is fed a post-surgery meal. In another embodiment, the treatment is characterized by an increase in contractility in the ileus. In another embodiment, the gastroparesis is post-operative ileus, or paralytic ileus. In another embodiment, the gastroparesis is not related to surgery. In another embodiment, the gastroparesis is related to diabetes, intestinal pseudo-obstruction, chronic constipation, dyspepsia, gastroesophageal reflux, emesis, paralytic gastroparesis, sepsis, or consumption of medications.

The invention also provides a method of inducing or increasing appetite or weight gain in a mammal in need thereof comprising administering to the mammal a polypeptide, wherein the polypeptide comprises the amino acid sequence of amino acid residues 44 to 95 of SEQ ID NO:2, or the amino acid sequence of amino acid residues 35 to 95 of SEQ ID NO: 2, or the amino acid sequenc of amino acid residues 44 to 85 of SEQ ID NO:5.

The invention also provides a method of modulating gastrointestinal contractility, gastric emptying or intestinal transit in a mammail in need thereof, comprising administering to the mammal a polypeptide, wherein the polypeptide comprises the amino amino acid sequence selected from: amino acid residues 44 to 95 and of SEQ ID NO:2 or the amino acid sequence of amino acid residues 35 to 95 of SEQ ID NO: 2. Within an embodiment, the polypeptide is administered orally, intraperitoneally, intravenously, intramuscularly, or sub cutaneously.

The invention also provides an isolated nucleic acid comprising the nucleic acid sequence as shown in SEQ ID NO:7.

The invention also provides a method of producing a polypeptide, comprising the step of culturing recombinant host cells that comprise an expression vector, wherein the expression vector comprises the isolated nucleic acid of as shown in SEQ ID NO:14, a transcription promoter, and a transcription terminator, wherein the promoter is operably linked with the nucleic acid, and wherein the nucleic acid is operably linked with the transcription terminator, and wherein the protein encoded by the nucleic acid is produced by the recombinant cell. The invention also provides the polypeptide produced by the method.

In another aspect the invention provides a method for modulating glucose levels in the blood by affecting insulin secretion from the pancreas.

These and other aspects of the invention will become evident upon reference to the following detailed description. In addition, various references are identified below and are incorporated by reference in their entirety.

### 2. Definitions

In the description that follows, a number of terms are used extensively. The following definitions are provided to facilitate understanding of the invention.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

An “anti-idiotype antibody” is an antibody that binds with the variable region domain of an immunoglobulin. In the present context, an anti-idiotype antibody binds with the variable region of an anti-Zsig98 antibody, and thus, an anti-idiotype antibody mimics an epitope of Zsig98.

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-Zsig98 monoclonal antibody fragment binds with an epitope of Zsig98.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain.

Due to the imprecision of standard analytical methods, molecular weights and lengths of polymers are understood to be approximate values. When such a value is expressed as “about” X or “approximately” X, the stated value of X will be understood to be accurate to ±10%.

### 3. Production of Human Zsig98 Genes

A Zsig98 gene can be obtained by synthesizing nucleic acid molecules using mutually priming long oligonucleotides and the nucleotide sequences described herein (see, for example, Ausubel (1995) at pages 8-8 to 8-9). Established techniques using the polymerase chain reaction provide the ability to synthesize DNA molecules at least two kilobases in length (Adang et al., Plant Molec. Biol. 21:1131 (1993), Bambot et al., PCR Methods and Applications 2:266 (1993), Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993), and Holowachuk et al., PCR Methods Appl. 4:299 (1995)). The nucleic acid molecules of the present invention can also be synthesized with “gene machines” using protocols such as the phosphoramidite method. Such methods are known in the art. For reviews on polynucleotide synthesis, see, for example, Glick and Pasternak, Molecular Biotechnology, Principles and Applications of Recombinant DNA (ASM Press 1994), Itakura et al., Annu. Rev. Biochem. 53:323 (1984), and Climie et al., Proc. Nat'l Acad. Sci. USA 87:633 (1990).

Cloning of 5′ flanking sequences also facilitates production of Zsig98 proteins by “gene activation,” as disclosed in U.S. Pat. No. 5,641,670. Briefly, expression of an endogenous Zsig98 gene in a cell is altered by introducing into the Zsig98 locus a DNA construct comprising at least a targeting sequence, a regulatory sequence, an exon, and an unpaired splice donor site. The targeting sequence is a Zsig98 5′ non-coding sequence that permits homologous recombination of the construct with the endogenous Zsig98 locus, whereby the sequences within the construct become operably linked with the endogenous Zsig98 coding sequence. In this way, an endogenous Zsig98 promoter can be replaced or supplemented with other regulatory sequences to provide enhanced, tissue-specific, or otherwise regulated expression.

### 4. Production of Zsig98 Gene Variants

The present invention provides a variety of nucleic acid molecules, including DNA and RNA molecules, which encode the Zsig98 polypeptides disclosed herein. Those skilled in the art will readily recognize that, in view of the degeneracy of the genetic code, considerable sequence variation is possible among these polynucleotide molecules. SEQ ID NOs:3 and 6 are a degenerate nucleotide sequences that encompasses all nucleic acid molecules that encode the Zsig98 polypeptides of SEQ ID NOs:2 and 5, respectively.

Different species can exhibit “preferential codon usage.” In general, see, Grantham et al., Nuc. Acids Res. 8:1893 (1980), Haas et al. Curr. Biol. 6:315 (1996), Wain-Hobson et al., Gene 13:355 (1981), Grosjean and Fiers, Gene 18:199 (1982), Holm, Nuc. Acids Res. 14:3075 (1986), Ikemura, J. Mol. Biol. 158:573 (1982), Sharp and Matassi, Curr. Opin. Genet. Dev. 4:851 (1994), Kane, Curr. Opin. Biotechnol. 6:494 (1995), and Makrides, Microbiol. Rev. 60:512 (1996).

The present invention further provides variant polypeptides and nucleic acid molecules that represent counterparts from other species (orthologs). These species include, but are not limited to mammalian, avian, amphibian, reptile, fish, insect and other vertebrate and invertebrate species. Of particular interest are Zsig98 polypeptides from other mammalian species, including porcine, ovine, bovine, canine, feline, equine, and other primate polypeptides. Orthologs of human Zsig98 can be cloned using information and compositions provided by the present invention in combination with conventional cloning techniques. For example, a cDNA can be cloned using mRNA obtained from a tissue or cell type that expresses Zsig98. Suitable sources of mRNA can be identified by probing northern blots with probes designed from the sequences disclosed herein. A library is then prepared from mRNA of a positive tissue or cell line.

Zsig98 polypeptides from varies species are shown herein to have an odd number of cysteines. Expression of recombinant Zsig98 can result in a heterologous mixture of proteins composed of intramolecular disulfide binding in multiple conformations. The separation of these forms can be difficult and laborious. It is therefore desirable to provide Zsig98 molecules having a consistent intramolecular disulfide bonding pattern upon expression and methods for refolding and purifying these preparations to maintain homogeneity. As such, the cysteine at position 69 of SEQ ID NO: 2 (position 59 of SEQ ID NO: 5, and position 64 of SEQ ID NO:9) may be substituted by tyrosine, phenylalanine, isoleucine, leucine, valine, serine, or tryptophan. These substitutions will be useful during expression, refolding, and purification since it limits the number of disulfide bond, and multimeric forms. It is unlikely that this substitution will have a significant effect on the receptor binding potential of the molecule.

Within certain embodiments of the invention, the isolated nucleic acid molecules can hybridize under stringent conditions to nucleic acid molecules comprising nucleotide sequences disclosed herein. For example, such nucleic acid molecules can hybridize under stringent conditions to nucleic acid molecules consisting of the nucleotide sequence of SEQ ID NO:1, to nucleic acid molecules consisting of the nucleotide sequence of nucleotides 66 to 161 of SEQ ID NO:1, to nucleic acid molecules consisting of the nucleotide sequence of nucleotides 288 to 389 of SEQ ID NO:1, to nucleic acid molecules consisting of the nucleotide sequence of SEQ ID NO:4, to nucleic acid molecules consisting of the nucleotide sequence of nucleotides 334 to 405 of SEQ ID NO:4, or to nucleic acid molecules consisting of nucleotide sequences that are the complements of such sequences. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions include temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions that influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below. This allows for the maximum rate of hybridization for DNA-DNA and DNA-RNA hybrids.

The ionic concentration of the hybridization buffer also affects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1×SSC: 0.15 M sodium chloride, 15 mM sodium citrate) or SSPE (1×SSPE: 1.8 M NaCl, 10 mM NaH₂PO₄, 1 mM EDTA, pH 7.7). By decreasing the ionic concentration of the buffer, the stability of the hybrid is increased. Typically, hybridization buffers contain from between 10 mM-1 M Na⁺. The addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As an illustration, a nucleic acid molecule encoding a variant Zsig98 polypeptide can be hybridized with a nucleic acid molecule having the nucleotide sequence of SEQ ID NO:1 (or its complement) at 42° C. overnight in a solution comprising 50% formamide, 5×SSC (1×SSC: 0.15 M sodium chloride and 15 mM sodium citrate), 50 mM sodium phosphate (pH 7.6), 5× Denhardt's solution (100× Denhardt's solution: 2% (w/v) Ficoll 400, 2% (w/v) polyvinylpyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formamide. Moreover, premixed hybridization solutions are available (e.g., EXPRESSHYB Hybridization Solution from CLONTECH Laboratories, Inc.), and hybridization can be performed according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. For example, nucleic acid molecules encoding particular variant Zsig98 polypeptides can remain hybridized with a nucleic acid molecule consisting of the nucleotide sequence of nucleotides 66 to 161 of SEQ ID NO:1, the nucleotide sequence of nucleotides 288 to 389 of SEQ ID NO:1, or their complements, following washing under stringent washing conditions, in which the wash stringency is equivalent to 0.5×-2×SSC with 0.1% SDS at 55-65° C., including 0.5×SSC with 0.1% SDS at 55° C., or 2×SSC with 0.1% SDS at 65° C. One of skill in the art can readily devise equivalent conditions, for example, by substituting SSPE for SSC in the wash solution.

Typical highly stringent washing conditions include washing in a solution of 0.1×-0.2×SSC with 0.1% sodium dodecyl sulfate (SDS) at 50-65° C. As an illustration, nucleic acid molecules encoding particular variant Zsig98 polypeptides can remain hybridized with a nucleic acid molecule consisting of the nucleotide sequence of nucleotides 66 to 161 of SEQ ID NO:1, the nucleotide sequence of nucleotides 288 to 389 of SEQ ID NO:1, or their complements, following washing under highly stringent washing conditions, in which the wash stringency is equivalent to 0.1×-0.2×SSC with 0.1% SDS at 50-65° C., including 0.1×SSC with 0.1% SDS at 50° C., or 0.2×SSC with 0.1% SDS at 65° C.

The present invention also provides isolated Zsig98 polypeptides that have a substantially similar sequence identity to the polypeptides of SEQ ID NO:2, or their orthologs. The term “substantially similar sequence identity” is used herein to denote polypeptides having 85%, 90%, 95% or greater than 95% sequence identity to the sequences shown in SEQ ID NO: 2, or its orthologs.

The present invention also contemplates Zsig98 variant nucleic acid molecules that can be identified using two criteria: a determination of the similarity between the encoded polypeptide with the amino acid sequence of SEQ ID NOs:2 or 5, and a hybridization assay, as described above. For example, certain Zsig98 gene variants include nucleic acid molecules (1) that remain hybridized with a nucleic acid molecule consisting of the nucleotide sequence of nucleotides 66 to 161 of SEQ ID NO:1, the nucleotide sequence of nucleotides 288 to 389 of SEQ ID NO:1, or their complements, following washing under stringent washing conditions, in which the wash stringency is equivalent to 0.5×-2×SSC with 0.1% SDS at 55-65° C., and (2) that encode a polypeptide having 85%, 90%, 95% or greater than 95% sequence identity to the amino acid sequence of SEQ ID NO:2. Alternatively, certain Zsig98 variant genes can be characterized as nucleic acid molecules (1) that remain hybridized with a nucleic acid molecule consisting of the nucleotide sequence of nucleotides 66 to 161 of SEQ ID NO:1, the nucleotide sequence of nucleotides 288 to 389 of SEQ ID NO:1, or their complements, following washing under highly stringent washing conditions, in which the wash stringency is equivalent to 0.1×-0.2×SSC with 0.1% SDS at 50-65° C., and (2) that encode a polypeptide having 85%, 90%, 95% or greater than 95% sequence identity to the amino acid sequence of SEQ ID NO:2.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative Zsig98. The FASTA algorithm is described by Pearson and Lipman, Proc. Nat'l Acad. Sci. USA 85:2444 (1988), and by Pearson, Meth. Enzymol. 183:63 (1990).

The present invention includes nucleic acid molecules that encode a polypeptide having a conservative amino acid change, compared with the amino acid sequence of SEQ ID NOs:2 or 5. That is, variants can be obtained that contain one or more amino acid substitutions of SEQ ID NOs:2 or 5, in which an alkyl amino acid is substituted for an alkyl amino acid in a Zsig98 amino acid sequence, an aromatic amino acid is substituted for an aromatic amino acid in a Zsig98 amino acid sequence, a sulfur-containing amino acid is substituted for a sulfur-containing amino acid in a Zsig98 amino acid sequence, a hydroxy-containing amino acid is substituted for a hydroxy-containing amino acid in a Zsig98 amino acid sequence, an acidic amino acid is substituted for an acidic amino acid in a Zsig98 amino acid sequence, a basic amino acid is substituted for a basic amino acid in a Zsig98 amino acid sequence, or a dibasic monocarboxylic amino acid is substituted for a dibasic monocarboxylic amino acid in a Zsig98 amino acid sequence.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

Variants of Zsig98 are characterized by having at least 70%, at least 80%, at least 85%, at least 90%, at least 95% or greater than 95% sequence identity to a corresponding amino acid sequence disclosed herein (i.e., SEQ ID NO:2 or SEQ ID NO:5), wherein the variation in amino acid sequence is due to one or more conservative amino acid substitutions.

Conservative amino acid changes in a Zsig98 gene can be introduced by substituting nucleotides for the nucleotides recited in SEQ ID NO:1 and SEQ ID NO:4, respectively. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol. 202:301 (1991), Chung et al., Science 259:806 (1993), and Chung et al., Proc. Nat'l Acad. Sci. USA 90:10145 (1993).

In a second method, translation is carried out in Xenopus oocytes by microinjection of mutated mRNA and chemically aminoacylated suppressor tRNAs (Turcatti et al., J. Biol. Chem. 271:19991 (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470 (1994). Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395 (1993)).

A limited number of non-conservative amino acids, amino acids that are not encoded by the genetic code, non-naturally occurring amino acids, and unnatural amino acids may be substituted for Zsig98 amino acid residues.

Essential amino acids in the polypeptides of the present invention can be identified according to procedures known in the art, such as site-directed mutagenesis or alanine-scanning mutagenesis (Cunningham and Wells, Science 244:1081 (1989), Bass et al., Proc. Nat'l Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity, such as the ability to bind to an antibody, to identify amino acid residues that are critical to the activity of the molecule. See also, Hilton et al., J. Biol. Chem. 271:4699 (1996).

The location of Zsig98 receptor binding domains can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306 (1992), Smith et al., J. Mol. Biol. 224:899 (1992), and Wlodaver et al., FEBS Lett. 309:59 (1992). Moreover, Zsig98 labeled with biotin or FITC can be used for expression cloning of Zsig98 receptor(s).

Multiple amino acid substitutions can be made and tested using known methods of mutagenesis and screening, such as those disclosed by Reidhaar-Olson and Sauer (Science 241:53 (1988)) or Bowie and Sauer (Proc. Nat'l Acad. Sci. USA 86:2152 (1989)). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832 (1991), Ladner et al., U.S. Pat. No. 5,223,409, Huse, international publication No. WO 92/06204, and region-directed mutagenesis (Derbyshire et al., Gene 46:145 (1986), and Ner et al., DNA 7:127, (1988)).

The present invention also includes “functional fragments” of Zsig98 polypeptides and nucleic acid molecules encoding such functional fragments. Routine deletion analyses of nucleic acid molecules can be performed to obtain functional fragments of a nucleic acid molecule that encodes a Zsig98 polypeptide. As an illustration, DNA molecules having the nucleotide sequence of SEQ ID NO:1 can be digested with Bal31 nuclease to obtain a series of nested deletions. The fragments are then inserted into expression vectors in proper reading frame, and the expressed polypeptides are isolated and tested for the ability to bind anti-Zsig98 antibodies. One alternative to exonuclease digestion is to use oligonucleotide-directed mutagenesis to introduce deletions or stop codons to specify production of a desired fragment. Alternatively, particular fragments of a Zsig98 gene can be synthesized using the polymerase chain reaction.

The present invention also contemplates functional fragments of a Zsig98 gene that have amino acid changes, compared with the amino acid sequence of SEQ ID NO:2 or SEQ ID NO:5. A variant Zsig98 gene can be identified on the basis of structure by determining the level of identity with the particular nucleotide and amino acid sequences disclosed herein. An alternative approach to identifying a variant gene on the basis of structure is to determine whether a nucleic acid molecule encoding a potential variant Zsig98 gene can hybridize to a nucleic acid molecule having the nucleotide sequence of SEQ ID NO:1 or SEQ ID NO:4, as discussed above.

The present invention also provides polypeptide fragments or peptides comprising an epitope-bearing portion of a Zsig98 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein.

Antigenic epitope-bearing peptides and polypeptides can contain at least four to ten amino acids, at least ten to fifteen amino acids, or about 15 to about 30 amino acids of SEQ ID NOs:2 or 5. Such epitope-bearing peptides and polypeptides can be produced by fragmenting a Zsig98 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

Regardless of the particular nucleotide sequence of a variant Zsig98 gene, the gene encodes a polypeptide that may be characterized by its ability to bind specifically to an anti-Zsig98 antibody.

For any Zsig98 polypeptide, including variants and fusion proteins, one of ordinary skill in the art can readily generate a fully degenerate polynucleotide sequence encoding that variant using the information set forth in Tables 1 and 2 above. Moreover, those of skill in the art can use standard software to devise Zsig98 variants based upon the nucleotide and amino acid sequences described herein. Accordingly, the present invention includes a computer-readable medium encoded with a data structure that provides at least one of the following sequences: SEQ ID NO:1, SEQ ID NO:2, SEQ ID NO:3, SEQ ID NO:4, SEQ ID NO:5, and SEQ ID NO:6. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

### 5. Production of Zsig98 Fusion Proteins

Fusion proteins of Zsig98 can be used to express a Zsig98 polypeptide or peptide in a recombinant host, and to isolate expressed Zsig98 polypeptides and peptides. One type of fusion protein comprises a peptide that guides a Zsig98 polypeptide from a recombinant host cell. To direct a Zsig98 polypeptide into the secretory pathway of a eukaryotic host cell, a secretory signal sequence (also known as a signal peptide, a leader sequence, prepro sequence or pre sequence) is provided in the Zsig98 expression vector. While the secretory signal sequence may be derived from Zsig98, a suitable signal sequence may also be derived from another secreted protein or synthesized de novo. The secretory signal sequence is operably linked to a Zsig98-encoding sequence such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences may be positioned elsewhere in the nucleotide sequence of interest (see, e.g., Welch et al., U.S. Pat. No. 5,037,743; Holland et al., U.S. Pat. No. 5,143,830).

Although the secretory signal sequence of Zsig98, or another protein produced by mammalian cells (e.g., tissue-type plasminogen activator signal sequence, as described, for example, in U.S. Pat. No. 5,641,655) is useful for expression of Zsig98 in recombinant mammalian hosts, a yeast signal sequence is preferred for expression in yeast cells. Examples of suitable yeast signal sequences are those derived from yeast mating phermone α-factor (encoded by the MFα1 gene), invertase (encoded by the SUC2 gene), or acid phosphatase (encoded by the PHO5 gene). See, for example, Romanos et al., “Expression of Cloned Genes in Yeast,” in DNA Cloning 2: A Practical Approach, 2^{nd} Edition, Glover and Hames (eds.), pages 123-167 (Oxford University Press 1995).

In bacterial cells, it is often desirable to express a heterologous protein as a fusion protein to decrease toxicity, increase stability, and to enhance recovery of the expressed protein. For example, Zsig98 can be expressed as a fusion protein comprising a glutathione S-transferase polypeptide. Glutathione S-transferease fusion proteins are typically soluble, and easily purifiable from E. coli lysates on immobilized glutathione columns. In similar approaches, a Zsig98 fusion protein comprising a maltose binding protein polypeptide can be isolated with an amylose resin column, while a fusion protein comprising the C-terminal end of a truncated Protein A gene can be purified using IgG-Sepharose. Established techniques for expressing a heterologous polypeptide as a fusion protein in a bacterial cell are described, for example, by Williams et al., “Expression of Foreign Proteins in E. coli Using Plasmid Vectors and Purification of Specific Polyclonal Antibodies,” in DNA Cloning 2: A Practical Approach, 2^{nd} Edition, Glover and Hames (Eds.), pages 15-58 (Oxford University Press 1995). In addition, commercially available expression systems are available. For example, the PINPOINT Xa protein purification system (Promega Corporation; Madison, Wis.) provides a method for isolating a fusion protein comprising a polypeptide that becomes biotinylated during expression with a resin that comprises avidin.

Structural analysis of Zsig98 polypeptides indicates that the active form of Zsig98 may be a homodimer, consisting of two chains of the mature Zsig98 protein, each chain comprising or consisting of amino acids 35 to 95 of SEQ ID NO:2. The Zsig98 polypeptides may be covalently linked by one or more intermolecular disulfide bonds or they may dimerize by one or more non-covalent hydrophobic and electrostatic interaction.

Peptide tags that are useful for isolating heterologous polypeptides expressed by either prokaryotic or eukaryotic cells include polyHistidine tags (which have an affinity for nickel-chelating resin), c-myc tags, calmodulin binding protein (isolated with calmodulin affinity chromatography), substance P, the RYIRS tag (which binds with anti-RYIRS antibodies), the Glu-Glu tag (which binds with anti-Glu-Glu antibodies), and the FLAG tag (which binds with anti-FLAG antibodies). See, for example, Luo et al., Arch. Biochem. Biophys. 329:215 (1996), Morganti et al., Biotechnol. Appl. Biochem. 23:67 (1996), and Zheng et al., Gene 186:55 (1997). Nucleic acid molecules encoding such peptide tags are available, for example, from Sigma-Aldrich Corporation (St. Louis, Mo.).

Another form of fusion protein comprises a Zsig98 polypeptide and an immunoglobulin heavy chain constant region, typically an F_{C} fragment, which contains two constant region domains and a hinge region but lacks the variable region. As an illustration, Chang et al., U.S. Pat. No. 5,723,125, describe a fusion protein comprising a human interferon and a human immunoglobulin Fc fragment. The C-terminal of the interferon is linked to the N-terminal of the Fc fragment by a peptide linker moiety. An example of a peptide linker is a peptide comprising primarily a T cell inert sequence, which is immunologically inert. In this fusion protein, a preferred Fc moiety is a human γ4 chain, which is stable in solution and has little or no complement activating activity. Accordingly, the present invention contemplates a Zsig98 fusion protein that comprises a Zsig98 polypeptide moiety and a human Fc fragment, wherein the C-terminus of the Zsig98 polypeptide moiety is attached to the N-terminus of the Fc fragment via a peptide linker.

In another variation, a Zsig98 fusion protein comprises an IgG sequence, a Zsig98 polypeptide moiety covalently joined to the amino terminal end of the IgG sequence, and a signal peptide that is covalently joined to the amino terminal of the Zsig98 polypeptide moiety, wherein the IgG sequence consists of the following elements in the following order: a hinge region, a CH₂ domain, and a CH₃ domain. Accordingly, the IgG sequence lacks a CH₁ domain. The Zsig98 polypeptide moiety displays a Zsig98 activity, such as the ability to bind with a Zsig98 receptor. This general approach to producing fusion proteins that comprise both antibody and nonantibody portions has been described by LaRochelle et al., EP 742830 (WO 95/21258).

Fusion proteins comprising a Zsig98 polypeptide moiety and an Fc moiety can be used, for example, as an in vitro assay tool. For example, the presence of a Zsig98 receptor in a biological sample can be detected using these Zsig98-antibody fusion proteins, in which the Zsig98 moiety is used to target the cognate receptor, and a macromolecule, such as Protein A or anti-Fc antibody, is used to detect the bound fusion protein-ligand complex. In addition, antibody-Zsig98 fusion proteins, comprising antibody variable domains, are useful as therapeutic proteins, in which the antibody moiety binds with a target antigen, such as a tumor associated antigen.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. General methods for enzymatic and chemical cleavage of fusion proteins are described, for example, by Ausubel (1995) at pages 16-19 to 16-25.

### 6. Production of Zsig98 Polypeptides

The polypeptides of the present invention, including full-length polypeptides, functional fragments, and fusion proteins, can be produced in recombinant host cells following conventional techniques. To express a Zsig98 gene, a nucleic acid molecule encoding the polypeptide must be operably linked to regulatory sequences that control transcriptional expression in an expression vector and then, introduced into a host cell. In addition to transcriptional regulatory sequences, such as promoters and enhancers, expression vectors can include translational regulatory sequences and a marker gene, which is suitable for selection of cells that carry the expression vector.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. As discussed above, expression vectors can also include nucleotide sequences encoding a secretory sequence that directs the heterologous polypeptide into the secretory pathway of a host cell. For example, a Zsig98 expression vector may comprise a Zsig98 gene and a secretory sequence derived from a Zsig98 gene or another secreted gene.

Zsig98 proteins of the present invention may be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 [Chasin et al., Som. Cell. Molec. Genet. 12:555 1986]), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658. As exemplary cell line of the pancreas to test the activity of zsig98 is CRL-1682, an human pancreas adenocarcinoma cell line, (ATCC, Manassas, Va.).

For a mammalian host, the transcriptional and translational regulatory signals may be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

The Zsig98 polypeptides of the present invention were expressed in the L293 mammalian cell line and purified. N-terminal sequencing revealed that the mature Zsig98 polypeptide from mammalian expression began with position 35 of SEQ ID NO: 2 (position 25 of SEQ ID NO: 5). Additional transcripts resulted which had the mature protein starting at position 37 of SEQ ID NO: 2, position 27 of SEQ ID NO: 5, and at postion 39 of SEQ ID NO: 2, position 29 of SEQ ID NO: 5. The Zsig98 gene is located on human chromosome 4p16.3.

Transcriptional regulatory sequences include a promoter region sufficient to direct the initiation of RNA synthesis. Suitable eukaryotic promoters include the promoter of the mouse metallothionein I gene (Hamer et al., J. Molec. Appl. Genet. 1:273 (1982)), the TK promoter of Herpes virus (McKnight, Cell 31:355 (1982)), the SV40 early promoter (Benoist et al., Nature 290:304 (1981)), the Rous sarcoma virus promoter (Gorman et al., Proc. Nat'l Acad. Sci. USA 79:6777 (1982)), the cytomegalovirus promoter (Foecking et al., Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)).

Alternatively, a prokaryotic promoter, such as the bacteriophage T3 RNA polymerase promoter, can be used to control Zsig98 gene expression in mammalian cells if the prokaryotic promoter is regulated by a eukaryotic promoter (Zhou et al., Mol. Cell. Biol. 10:4529 (1990), and Kaufman et al., Nucl. Acids Res. 19:4485 (1991)).

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase, which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Zsig98 polypeptides can also be produced by cultured mammalian cells using a viral delivery system. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161 (1994), and Douglas and Curiel, Science & Medicine 4:44 (1997)). Advantages of the adenovirus system include the accommodation of relatively large DNA inserts, the ability to grow to high-titer, the ability to infect a broad range of mammalian cell types, and flexibility that allows use with a large number of available vectors containing different promoters.

Zsig98 genes may also be expressed in other higher eukaryotic cells, such as avian, fungal, insect, yeast, or plant cells. The baculovirus system provides an efficient means to introduce cloned Zsig98 genes into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. A second method of making recombinant baculovirus utilizes a transposon-based system described by Luckow (Luckow, et al., J. Virol. 67:4566 (1993)). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the Zsig98 polypeptide into a baculovirus genome maintained in E. coli as a large plasmid called a “bacmid.” See, Hill-Perkins and Possee, J. Gen. Virol. 71:971 (1990), Bonning, et al., J. Gen. Virol. 75:1551 (1994), and Chazenbalk, and Rapoport, J. Biol. Chem. 270:1543 (1995). In addition, transfer vectors can include an in-frame fusion with DNA encoding an epitope tag at the C- or N-terminus of the expressed Zsig98 polypeptide, for example, a Glu-Glu epitope tag (Grussenmeyer et al., Proc. Nat'l Acad. Sci. 82:7952 (1985)). Using a technique known in the art, a transfer vector containing a Zsig98 gene is transformed into E. coli, and screened for bacmids, which contain an interrupted lacZ gene indicative of recombinant baculovirus. The bacmid DNA containing the recombinant baculovirus genome is then isolated using common techniques.

Fungal cells, including yeast cells, can also be used to express the genes described herein. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). A suitable vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11-23 (1998), and in international publication Nos. WO 97/17450, WO 97/17451, WO 98/02536, and WO 98/02565. DNA molecules for use in transforming P. methanolica will commonly be prepared as double-stranded, circular plasmids, which can be linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A suitable selectable marker for use in Pichia methanolica is a P. methanolica ADE2 gene, which encodes phosphoribosyl-5-aminoimidazole carboxylase (AIRC; EC 4.1.1.21), and which allows ade2 host cells to grow in the absence of adenine. For large-scale, industrial processes where it is desirable to minimize the use of methanol, it is possible to use host cells in which both methanol utilization genes (AUG1 and AUG2) are deleted. For production of secreted proteins, host cells can be used that are deficient in vacuolar protease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Expression vectors can also be introduced into plant protoplasts, intact plant tissues, or isolated plant cells. Methods for introducing expression vectors into plant tissue include the direct infection or co-cultivation of plant tissue with Agrobacterium tumefaciens, microprojectile-mediated delivery, DNA injection, electroporation, and the like. See, for example, Horsch et al., Science 227:1229 (1985), Klein et al., Biotechnology 10:268 (1992), and Miki et al., “Procedures for Introducing Foreign DNA into Plants,” in Methods in Plant Molecular Biology and Biotechnology, Glick et al. (eds.), pages 67-88 (CRC Press, 1993).

Alternatively, Zsig98 genes can be expressed in prokaryotic host cells. Suitable promoters that can be used to express Zsig98 polypeptides in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277 (1987), Watson et al., Molecular Biology of the Gene, 4th Ed. (Benjamin Cummins 1987), and by Ausubel et al. (1995).

Suitable prokaryotic hosts include E. coli and Bacillus subtilus. Suitable strains of E. coli include BL21(DE3), BL21(DE3)pLysS, BL21(DE3)pLysE, DH1, DH4I, DH5, DH5I, DH5IF′, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (ed.), Molecular Biology Labfax (Academic Press 1991)). Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)).

When expressing a Zsig98 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995), Ward et al., “Genetic Manipulation and Expression of Antibodies,” in Monoclonal Antibodies: Principles and Applications, page 137 (Wiley-Liss, Inc. 1995), and Georgiou, “Expression of Proteins in Bacteria,” in Protein Engineering: Principles and Practice, Cleland et al. (eds.), Chapter 4, starting at page 101 (John Wiley & Sons, Inc. 1996), and Rudolph, “Successful Refolding on an Industrial Scale”, Chapter 10).

Standard methods for introducing expression vectors into bacterial, yeast, insect, and plant cells are provided, for example, by Ausubel (1995).

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et al. (eds.), pages 59-92 (Oxford University Press 1995). Established methods for isolating recombinant proteins from a baculovirus system are described by Richardson (ed.), Baculovirus Expression Protocols (The Humana Press, Inc. 1995).

As an alternative, polypeptides of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), and Severinov and Muir, J. Biol. Chem. 273:16205 (1998)).

Peptides and proteins that contain multiple disulfide bonds can be oxidized in the presence of mixtures of low molecular weight disulfides and the corresponding free thiols by thiol-disulfide exchange which facilitates the reshuffling of incorrectly paired disulfides to the native state. The most commonly used systems are mixtures of oxidized and reduced glutathione, cysteine, cysteamine, or 2-mercaptoethanol. As with all solution oxidations, yields are improved under high dilution conditions that reduce the potential levels of oligomerization. Zsig98 proteins were refolded as shown in Example 7.

The refolded protein as shown in Example 7 contained multimers and dimers. Because the heterogeneity of forms is believed to be a result of multiple intramolecular disulfide bonding patterns, specific embodiments of the present invention include mutations to the cysteine residues within the wildtype Zsig98 sequences. Thus, the present invention provides polynucleotide molecules, including DNA and RNA molecules, that encode Cysteine mutants of Zsig98 that result in expression of a recombinant Zsig98 preparation that is a homogeneous preparation. For the purposes of this invention, a homogeneous preparation of Zsig98 is a preparation which comprises at least 98% of a single intramolecular disulfide bonding pattern in the purified polypeptide. In other embodiments, the single disulfide conformation in a preparation of purified polypeptide is at 99% homogeneous. In general, these Cysteine mutants will maintain some biological activity of the wildtype Zsig98, as described herein. For example, the molecules of the present invention can bind to the Zsig98 receptor with some specificity. Generally, a ligand binding to its cognate receptor is specific when the KD falls within the range of 100 nM to 100 pM. Specific binding in the range of 100 mM to 10 nM KD is low affinity binding. Specific binding in the range of 2.5 pM to 100 pM KD is high affinity binding. In another example, biological activity of Zsig98 Cysteine mutants is present when the molecules are capable of some level of activity associated with wildtype Zsig98 as described in detail herein.

The Zsig98 Cysteine mutants as described herein include the polypeptide where the cysteine at position 69 of SEQ ID NO: 2 has been mutated to another amino acid with similar properties, but that does not form a disulfide bond with other cyteines. For example, the cyteine at position 69 of SEQ ID NO: 2 or postion 59 of SEQ ID NO: 5 cn be mutated to a serine, threonine, alanine, isoleucine, leucine, or valine. In addition, when human Zsig98 is expressed in E. coli, an N-terminal or amino-terminal Methionine may be needed for expression.

Peptides and polypeptides of the present invention comprise at least six, at least nine, or at least 15 contiguous amino acid residues of SEQ ID NOs:2 and 5. Illustrative polypeptides of Zsig98, for example, include 15 contiguous amino acid residues of amino acids 35 to 95 of SEQ ID NO:2. Within certain embodiments of the invention, the polypeptides comprise 20, 30, 40, 50, 75, or more contiguous residues of SEQ ID NOs:2 or 5. Nucleic acid molecules encoding such peptides and polypeptides are useful as polymerase chain reaction primers and probes.

The present invention contemplates compositions comprising a peptide or polypeptide described herein. Such compositions can further comprise a carrier. The carrier can be a conventional organic or inorganic carrier. Examples of carriers include water, buffer solution, alcohol, propylene glycol, macrogol, sesame oil, corn oil, and the like.

An expression vector containing a GLU-GLU tag can be designed and prepared to express Zsig98cee polypeptides in insect cells.

### 7. Isolation of Zsig98 Polypeptides

The polypeptides of the present invention can be purified to at least about 80% purity, to at least about 90% purity, to at least about 95% purity, or even greater than 95% purity with respect to contaminating macromolecules, particularly other proteins and nucleic acids, and free of infectious and pyrogenic agents. The polypeptides of the present invention can also be purified to a pharmaceutically pure state, which is greater than 99.9% pure. In certain preparations, a purified polypeptide is substantially free of other polypeptides, particularly other polypeptides of animal origin.

Fractionation and/or conventional purification methods can be used to obtain preparations of Zsig98 purified from natural sources, and recombinant Zsig98 polypeptides and fusion Zsig98 polypeptides purified from recombinant host cells. In general, ammonium sulfate precipitation and acid or chaotrope extraction may be used for fractionation of samples. Exemplary purification steps may include hydroxyapatite, size exclusion, FPLC a n d reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method for polypeptide isolation and purification is a matter of routine design and is determined in part by the properties of the chosen support. See, for example, Affinity Chromatography: Principles & Methods (Pharmacia LKB Biotechnology 1988), and Doonan, Protein Purification Protocols (The Humana Press 1996).

Additional variations in Zsig98 isolation and purification can be devised by those of skill in the art. For example, anti-Zsig98 antibodies, obtained as described below, can be used to isolate large quantities of protein by immunoaffinity purification. Moreover, methods for binding receptors to ligand polypeptides, such as Zsig98 bound to support media are well known in the art.

The polypeptides of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher, (ed.), Meth. Enzymol. 182:529 (1990)). Within additional embodiments of the invention, a fusion of the polypeptide of interest and an affinity tag (e.g., maltose-binding protein, an immunoglobulin domain) may be constructed to facilitate purification.

Zsig98 polypeptides or fragments thereof may also be prepared through chemical synthesis, as described above. Zsig98 polypeptides may be monomers or multimers; glycosylated or non-glycosylated; pegylated or non-pegylated; and may or may not include an initial methionine amino acid residue.

### 8. Zsig98 Analogs

As described above, the disclosed polypeptides can be used to construct Zsig98 variants. These polypeptides can be used to identify Zsig98 analogs. One type of Zsig98 analog mimics Zsig98 by binding with a Zsig98 receptor. Such an analog is considered to be a Zsig98 agonist if the binding of the analog with a Zsig98 receptor stimulates a response by a cell that expresses the receptor. On the other hand, a Zsig98 analog that binds with a Zsig98 receptor, but does not stimulate a cellular response, may be a Zsig98 antagonist. Such an antagonist may diminish Zsig98 or Zsig98 agonist activity, for example, by a competitive or non-competitive binding of the antagonist to the Zsig98 receptor.

One general class of Zsig98 analogs are agonists or antagonists having an amino acid sequence that has at least one mutation, deletion (amino- or carboxyl-terminus), or substitution of the amino acid sequences disclosed herein. Another general class of Zsig98 analogs is provided by anti-idiotype antibodies, and fragments thereof, as described below. Moreover, recombinant antibodies comprising anti-idiotype variable domains can be used as analogs (see, for example, Monfardini et al., Proc. Assoc. Am. Physicians 108:420 (1996)). Since the variable domains of anti-idiotype Zsig98 antibodies mimic Zsig98, these domains can provide either Zsig98 agonist or antagonist activity. As an illustration, Lim and Langer, J. Interferon Res. 13:295 (1993), describe anti-idiotypic interferon-α antibodies that have the properties of either interferon-α agonists or antagonists.

A third approach to identifying Zsig98 analogs is provided by the use of combinatorial libraries. Methods for constructing and screening phage display and other combinatorial libraries are provided, for example, by Kay et al., Phage Display of Peptides and Proteins (Academic Press 1996), Verdine, U.S. Pat. No. 5,783,384, Kay, et. al., U.S. Pat. No. 5,747,334, and Kauffman et al., U.S. Pat. No. 5,723,323.

The activity of a Zsig98 polypeptide, agonist, or antagonist can be determined using a standard cell proliferation or differentiation assay. For example, assays measuring proliferation include such assays as chemosensitivity to neutral red dye, incorporation of radiolabeled nucleotides, incorporation of 5-bromo-2′-deoxyuridine in the DNA of proliferating cells, and use of tetrazolium salts (Mosmann, J. Immunol. Methods 65:55 (1983); Porstmann et al., J. Immunol. Methods 82:169 (1985); Alley et al., Cancer Res. 48:589 (1988); Cook et al., Analytical Biochem. 179:1 (1989); Marshall et al., Growth Reg. 5:69 (1995); Scudiero et al., Cancer Res. 48:4827 (1988); Cavanaugh et al., Investigational New Drugs 8:347 (1990)). Assays measuring differentiation include, for example, measuring cell-surface markers associated with stage-specific expression of a tissue, enzymatic activity, functional activity or morphological changes (Raes, Adv. Anim. Cell Biol. Technol. Bioprocesses, pages 161-171 (1989; Watt, FASEB, 5:281 (1991); Francis, Differentiation 57:63 (1994)). Assays can be used to measure other cellular responses, that include, chemotaxis, adhesion, changes in ion channel influx, regulation of second messenger levels and neurotransmitter release. Such assays are well known in the art (see, for example, Chayen and Bitensky, Cytochemical Bioassays: Techniques & Applications (Marcel Dekker 1983)).

The effect of a variant Zsig98 polypeptide, such as Zsig98, as well as agonists, fragments, variants and/or chimeras thereof, can also be determined by observing contractility of tissues, including gastrointestinal tissues, with a tensiometer that measures contractility and relaxation in tissues (see, for example, Dainty et al., J. Pharmacol. 100:767 (1990); Rhee et al., Neurotox. 16:179 (1995); Anderson, Endocrinol. 114:364 (1984); Downing, and Sherwood, Endocrinol. 116:1206 (1985)). For example, methods for measuring vasodilatation of aortic rings are well known in the art. As an illustration, aortic rings are removed from four-month old Sprague Dawley rats and placed in a buffer solution, such as modified Krebs solution (118.5 mM NaCl, 4.6 mM KCl, 1.2 mM MgSO₄.7H₂O, 1.2 mM KH₂PO₄, 2.5 mM CaCl₂.2H₂O, 24.8 mM NaHCO₃ and 10 mM glucose). One of skill in the art would recognize that this method can be used with other animals, such as rabbits, other rat strains, Guinea pigs, and the like. The rings are then attached to an isometric force transducer (Radnoti Inc., Monrovia, Calif.) and the data are recorded with a Ponemah physiology platform (Gould Instrument systems, Inc., Valley View, Ohio) and placed in an oxygenated (95% O₂, 5% CO₂) tissue bath containing the buffer solution. The tissues are adjusted to one gram resting tension and allowed to stabilize for about one hour before testing. The integrity of the rings can be tested with norepinepherin (Sigma Co.; St. Louis, Mo.) and carbachol, a muscarinic acetylcholine agonist (Sigma Co.). After integrity is checked, the rings are washed three times with fresh buffer and allowed to rest for about one hour. To test a sample for vasodilatation, or relaxation of the aortic ring tissue, the rings are contracted to two grams tension and allowed to stabilize for fifteen minutes. A Zsig98 polypeptide sample is then added to one, two, or three of the four baths, without flushing, and tension on the rings recorded and compared to the control rings containing buffer only. Enhancement or relaxation of contractility by Zsig98 polypeptides, their agonists and antagonists is directly measured by this method, and it can be applied to other contractile tissues such as gastrointestinal tissues.

As another example, the effects of Zsig98 can be tested in a standard guinea pig ileum organ bath. The organ bath system is a standard method used to measure contractility in isolated tissue, and the guinea pig ileum is routinely used for recording contractile responses in the intestine ex vivo (Thomas E., et al., Mol Pharmacol 44:102-10, 1993). Because the components of the enteric nervous system are located entirely within the gut, it may be removed from the brain and the spinal cord and its reflex behaviors studied. The classical response observed in gastrointestinal tissue from guinea pig intestinal ileum is longitudinal contraction by smooth muscle fibers orientated along the long axis of the gut.

The effect of a variant Zsig98 polypeptide, such as Zsig98, as well as agonists, fragments, variants and/or chimeras thereof, on gastric motility would typically be measured in the clinical setting as the time required for gastric emptying and subsequent transit time through the gastrointestinal tract. Gastric emptying scans are well known to those skilled in the art, and briefly comprise use of an oral contrast agent, such as barium, or a radiolabeled meal. Solids and liquids can be measured independently. Generally, a test food or liquid is radiolabeled with an isotope (e.g., ^{99m}Tc), and after ingestion or administration, transit time through the gastrointestinal tract and gastric emptying are measured by visualization using gamma cameras (Meyer et al., Am. J. Dig. Dis. 21:296 (1976); Collins et al., Gut 24:1117 (1983); Maughan et al., Diabet. Med. 13:S6 (1996), and Horowitz et al., Arch. Intern. Med. 145:1467 (1985)). The oral administration of phenol red (test meal) to measure gastric emptying and intestinal transit in rodents is a well-documented model (Martinez V, Cuttitta F, Tache Y 1997 Endocrinology 138:3749-3755). Briefly, animals are deprived of food for 18 hours but allowed free access to water. Animals receive oral administration of 0.15 ml of test meal, consisting of a 1.5% aqueous methylcellulose solution containing a non-absorbable dye, 0.05% phenol red (50 mg/100 ml Sigma Chemical Company Catalogue # P4758). The effects of Zsig98 on gastric emptying in an in vivo mouse model are shown in Examples 4, 8, 21, and 22. Additional studies can be performed before and after the administration of a promotility agent to quantify the efficacy of the Zsig98 polypeptide.

Radiolabeled or affinity labeled Zsig98 polypeptides can also be used to identify or to localize Zsig98 receptors in a biological sample (see, for example, Deutscher (ed.), Methods in Enzymol., vol. 182, pages 721-37 (Academic Press 1990); Brunner et al., Ann. Rev. Biochem. 62:483 (1993); Fedan et al., Biochem. Pharmacol. 33:1167 (1984)). Also see, Varthakavi and Minocha, J. Gen. Virol. 77:1875 (1996), who describe the use of anti-idiotype antibodies for receptor identification.

### 9. Production of Antibodies to Zsig98 Proteins

Antibodies to a Zsig98 polypeptide can be obtained, for example, using the product of a Zsig98 expression vector or Zsig98 isolated from a natural source as an antigen. Particularly useful anti-Zsig98 antibodies “bind specifically” with Zsig98, respectively. Antibodies are considered to be specifically binding if the antibodies exhibit at least one of the following two properties: (1) antibodies bind to Zsig98 with a threshold level of binding activity, and (2) antibodies do not significantly cross-react with polypeptides related to Zsig98.

With regard to the first characteristic, antibodies specifically bind if they bind to a Zsig98 polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660 (1949)). With regard to the second characteristic, antibodies do not significantly cross-react with related polypeptide molecules, for example, if they detect Zsig98, but not known polypeptides (e.g., known Wnt inhibitors) using a standard Western blot analysis.

Anti-Zsig98 antibodies can be produced using antigenic Zsig98 epitope-bearing peptides and polypeptides. Antigenic epitope-bearing peptides and polypeptides of the present invention contain a sequence of at least four, or between 15 to about 30 amino acids contained within SEQ ID NOs:2 or 5. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that bind with Zsig98. It is desirable that the amino acid sequence of the epitope-bearing peptide is selected to provide substantial solubility in aqueous solvents (i.e., the sequence includes relatively hydrophilic residues, while hydrophobic residues are preferably avoided). Moreover, amino acid sequences containing proline residues may be also be desirable for antibody production.

As an illustration, potential antigenic sites in Zsig98 were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

The results of this analysis indicated that suitable antigenic peptides of Zsig98 include the following segments of the amino acid sequence of SEQ ID NO:2: amino acids 1 to 6 (“antigenic peptide 1”), amino acids 20 to 31 (“antigenic peptide 2”), amino acids 33 to 40 (“antigenic peptide 3”), amino acids 50 to 66 (“antigenic peptide 4”), and amino acids 71 to 77 (“antigenic peptide 5”). The present invention contemplates the use of any one of antigenic peptides 1 to 6 to generate antibodies to Zsig98. The present invention also contemplates polypeptides comprising at least one of antigenic peptides 1 to 5.

Polyclonal antibodies to recombinant Zsig98 protein or to Zsig98 isolated from natural sources can be prepared using methods well-known to those of skill in the art. See, for example, Green et al., “Production of Polyclonal Antisera,” in Immunochemical Protocols (Manson, ed.), pages 1-5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a Zsig98 polypeptide can be increased through the use of an adjuvant, such as alum (aluminum hydroxide) or Freund's complete or incomplete adjuvant. Polypeptides useful for immunization also include fusion polypeptides, such as fusions of Zsig98 or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an anti-Zsig98 antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990). An exemplary polyclonal antibody can be made to the amino acid sequence of the peptide from amino acid 69 to amino acid 95 of SEQ ID NO: 2.

Alternatively, monoclonal anti-Zsig98 antibodies can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1-2.6.7 (John Wiley & Sons 1991) [“Coligan”], Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a Zsig98 gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an anti-Zsig98 antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-Zsig98 antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al., in Methods in Enzymology Vol. 1, page 422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991) (also see, Bird et al., Science 242:423 (1988), Ladner et al., U.S. Pat. No. 4,946,778, Pack et al., Bio/Technology 11:1271 (1993), and Sandhu, supra).

As an illustration, a scFV can be obtained by exposing lymphocytes to Zsig98 polypeptide in vitro, and selecting antibody display libraries in phage or similar vectors (for instance, through use of immobilized or labeled Zsig98 protein or peptide). Genes encoding polypeptides having potential Zsig98 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides, which interact with a known target that can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the Zsig98 sequences disclosed herein to identify proteins which bind to Zsig98.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-Zsig98 antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399-434 (John Wiley & Sons, Inc. 1996), and by Queen et al., U.S. Pat. No. 5,693,762 (1997).

Polyclonal anti-idiotype antibodies can be prepared by immunizing animals with anti-Zsig98 antibodies or antibody fragments, using standard techniques. See, for example, Green et al., “Production of Polyclonal Antisera,” in Methods In Molecular Biology: Immunochemical Protocols, Manson (ed.), pages 1-12 (Humana Press 1992). Also, see Coligan at pages 2.4.1-2.4.7. Alternatively, monoclonal anti-idiotype antibodies can be prepared using anti-Zsig98 antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875 (1996).

### 10. Therapeutic Uses of Zsig98 Polypeptides

The present invention includes the use of proteins, polypeptides, and peptides having Zsig98 activity (such as Zsig98 polypeptides, Zsig98 analogs, active Zsig98 anti-idiotype antibodies, and Zsig98 fusion proteins) to a subject, which lacks an adequate amount of this polypeptide. The present invention contemplates both veterinary and human therapeutic uses. Illustrative subjects include mammalian subjects, such as farm animals, domestic animals, and human patients.

Zsig98 polypeptides, such as Zsig98, as well as agonists, fragments, variants and/or chimeras thereof, and antagonists thereof are useful in diseases characterized by dysfunction of the gastrointestinal tract due to limited contractility, gastric emptying, and/or increased contractility.

Dysfunction of the gastrointestinal tract due to limited contractility and/or gastric emptying is a characteristic of diseases and disorders including, but not limited to, post-operative ileus, post-partum ileus, chronic constipation, dyspepsia, intestinal pseudo-obstruction, gastroparesis, diabetic gastroparesis, gastroesophageal reflux, emesis, use or consumption of opiods and/or narcotics, muscular dystrophy, progressive systemic sclerosis, infectious diarrhea, and paralytic gastroparesis.

Postoperative inhibition of gastrointestinal motility (postoperative ileus) is induced by laparotomy and intra-abdominal procedures. The transient inhibition of gastrointestinal motility occurring in humans mainly in the stomach and the colon may last for several days and can considerably contribute to a patient's postoperative discomfort. Oral food intake may be delayed until post operative ileus has been resolved, and prolonged nasogastric suction or, in rare cases, even relaparatomy becomes necessary (Livingston, E. et al., Post operataive ileu. Dig. Dis Sci 35:121-132, 1990). Zsig98 polypeptides, such as Zsig98, as well as agonists, fragments, variants and/or chimeras thereof, can be used to stimulate gastrointestinal contractility in patients after surgery. For example, patients who have disorders such as, post-surgical gastroparesis, including post-operative ileus, are good candidates for administration of Zsig98 polypeptides, such as Zsig98, as well as agonists, fragments, variants and/or chimeras thereof.

Post-operative ileus (POI) is a condition of reduced intestinal tract motility, including delayed gastric emptying, that occurs as a result of disrupted muscle tone following surgery. It is especially problematic following abdominal surgery. The problem may arise from the surgery itself, from the residual effects of anesthetic agents, and particularly, from pain-relieving narcotic and opiate drugs used during and after surgery.

Post-operative ileus reduces gastrointestinal motility, which also may delay the absorption of drugs administered orally. Reduced intestinal motility following surgery is a major cause of extended hospital stays, which are extremely expensive and result in an increased chance of developing other complications. Extended durations of POI may require the use of parenteral nutrition, which also is expensive. With the increasing cost of medical care, the expenses associated with hospital stays and parenteral feeding are expected to increase even further.

The acute nature of this condition provides an opportunity to treat with Zsig98 polypeptides, such as Zsig98, as well as agonists, fragments, variants and/or chimeras thereof. As a protein expressed from lactatig breast tissue, Zsig98 may be active if administered orally. Moreover, since most oral drugs would be counter-indicated during POI, a drug administered subcutaneously, intramuscularly, or intravenously, such as Zsig98 would be beneficial. “Prokinetics” have been found to alleviate the symptoms associated with POI. Currently there are very few drugs that can effectively treat POI, and those that are available have side effects, cannot be taken with other medications, and/or are administered orally. Zsig98 may be effective treating POI.

The effect of a Zsig98 polypeptide, such as Zsig98, as well as agonists, fragments, variants and/or chimeras thereof, on POI can be measured in an in vivo model by administering it orally (p.o.), intraperitoneally (i.p.), intraveneously (i.v.), subcutaneously (s.c.), or intramuscularly (i.m.) to fasted animals at an appropriate time prior to or following a laparotomy and cecal manipulation performed under anesthesia. One of the models listed below may then be used to assess extent of gastric emptying and/or intestinal transit at times ranging from 10 to 180 minutes after removal of the anesthetic. In dog models, this time may be greater (up to 50 h). Using several post-surgical time points allows an estimate of the effects of surgery on gastric emptying and transit along much of the gastrointestinal tract. This model has been used extensively to evaluate the efficacy of prokinetic drugs on gastric emptying and/or intestinal transit as a result of POI (e.g. Martinez, Rivier, and Tache. J. Pharmacol. Experimental. Therap. 290:629 (1999) and Furuta et al. Biol. Pharm. Bull. 25:103-1071 (2002)).

Additionally, Zsig98 polypeptides, such as Zsig98, as well as agonists, fragments, variants and/or chimeras thereof, can be used to prevent POI. In this scenario, the Zsig98 polypeptides, such as Zsig98, as well as agonists, fragments, variants and/or chimeras thereof, are administered to the patient pre-operatively.

Diabetic gastroparesis is paralysis of the stomach brought about by a motor abnormality in the stomach, as a complication of both type I and type II diabetes. It is characterized by delayed gastric emptying, post-prandial distention, nausea and vomiting. In diabetes, it is thought to be due to a neuropathy, though it is also associated with loss of interstitial cells of Cajal (ICC), which are the “pacemaker cells” of the gut.

Patients who have diabetes mellitus may also have disorders related to gastric emptying. For example, a patient who has had diabetes mellitus for at least five years may have a prevalence of significant delay in gastric emptying of >50% (Horowitz, M, et al., J. Gastoenterology Hepatology: 1:97-113, 1986). Gastric neuromuscular dysfunction occurs in up to 30-50% of patients after 10 years of type I or type 2 diabetes (Koch K., et al., Dig Dis Sci 44:1061-1075, 1999). Zsig98, and/or their agonists may also be used as treatment for diabetic patients.

The often-acute nature of the episodes of diabetic gastroparesis provides an opportunity to treat with Zsig98 polypeptides, such as Zsig98, as well as agonists, fragments, variants and/or chimeras thereof. “Prokinetics” have been found to alleviate the delayed gastric emptying associated with diabetic gastroparesis. Currently there are very few drugs that can effectively treat diabetic gastroparesis, and those that are available have side effects and/or cannot be taken with other medications. Oral drugs may not be tolerated during severe episodes, and thus, would require intravenous administration of a prokinetic.

The spontaneously diabetic NOD/LtJ mouse (available from Jackson Laboratories) develops delayed gastric emptying, impaired electrical pacemaking, and reduced motor neurotransmission. This is described in Watkins et al. J. Clin. Invest. 106:373-384 (2000). This strain also appears to have defects in interstitial cells of Cajal (ICC) networks that are associated with impaired motility. Streptozotocin treatment of rats and mice is a well-recognized and acceptable method to induce diabetes; these animals are characterized by impaired gastric emptying and intestinal transit, and thus, show symptoms of diabetic gastroparesis (Yamano et al. Naunyn-Schmiedeberg's Arch. Pharmacol. 356: 145-150 (1997) and Watkins et al. J. Clin. Invest. 106:373-384 (2000)). The ability of Zsig98 polypeptide, such as Zsig98, as well as agonists, fragments, variants and/or chimeras thereof, (administered via p.o., i.v., i.p., s.c., or i.m.) to improve the impaired gastric emptying and intestinal transit associated with the diabetes, can also be measured by one of the models described below.

Exocrine cells of the pancreas are important for the production of necessary enzymes involved in digestion. Persons defective in the Zsig98 gene may be unable to properly digest food and and nutrients. Polynucleotides of Zsig98 may be useful in treating a defective pancreatic specific gene by gene therapy. Likewise, polypeptides of the present invention could be administered to a mammal as replacement therapy for a defective digestive enzyme.

Zsig98 gene may be useful to as a probe to identify humans who have a defective pancreatic or colonic Zsig98 gene. The strong expression of Zsig98 in pancreas suggests that Zsig98 polynucleotides or polypeptides are down-regulated in tumor, malignant, or immune-responding tissues. Thus, polynucleotides and polypeptides of Zsig98, and mutations to them, can be used a indicators of pancreatic cancer, and disease, in diagnosis.

As a protein showing strong expression in the pancreas additional applications are to modulate gastric secretion in the treatment of acute pancreatitis and gastrointestinal disorders. Zsig98 expression was observed in both alpha and beta like cell lines by PCR, and was most highly expressed in NIT-1 cells. The NIT-1 cell line is a pancreatic beta cell line associated with insulioma. See Hamaguchi, K. et al. Diabetes 40:842-849, 1991; ATCC catalog number CRL-2055™. In addition, Zsig98 was abserved by PCR in an OC10b cell line. As such, the Zsig98 molecules of the present invention can be used to modulate blood sugar or metabolism. For example, Zsig98 polypeptides can be used to stimulate secretion of insulin in response to an increase in blood glucose. Similarly, Zsig98 polypeptide antagonists may be used to inhibit the secretion of insulin in conditions of hyperinsulinemia.

The expression of Zsig98 was deterimined to be mostly in tissues of the digestive tract by PCR. In addition, Zsig98 was expressed in hypothalamic cell line. The hypothalamus is critical for neuro-endocrine control of feeding, metabolism and islet cell function. See Table 1.

TABLE 1
Zsig98 Expression by PCR

| | | Sample | zsig98 PCR | | |
|---|---|---|---|---|---|
| | | array 121.01 | Results | type | biological system |
| A | 1 | Y-1 | −/− | adrenal tumor cell line | Endocrine |
| C | 1 | P53 hypo #3 | ++ | P53 −/− hypothalamus cell line, | Nervous |
| | | | | growth condition #3 | |
| E | 1 | alpha TC1.9 | ++ | pancreas cell line | Digestive (and Endocrine) |
| G | 1 | pid24 | −/− | pancreas cell line | Digestive (and Endocrine) |
| A | 2 | Y1-K26 | −/− | adrenal tumor cell line | Endocrine |
| C | 2 | P53 hypo #4 | ++ | P53 −/− #4 hypothalamus cell line, | Nervous |
| | | | | growth condition #4 | |
| E | 2 | NIT-1 | +++ | pancreas beta-cell line | Digestive (and Endocrine) |
| G | 2 | pid6 | + | pancreas cell line | Digestive (and Endocrine) |
| A | 3 | 229 | + | osteoblast cell line | Skeletal |
| C | 3 | TCMK-1 | + | kidney epithelial cell line | Urinary (and Endocrine) |
| E | 3 | P53DN1 | + | pancreas cell line | Digestive (and Endocrine) |
| G | 3 | pik10 | + | pancreas cell line | Digestive (and Endocrine) |
| A | 4 | 245 | +/− | osteoblast cell line | Skeletal |
| C | 4 | AML | −/− | acute myeloid leukaemia cell line | Digestive |
| E | 4 | P53DN1-15 | −/− | pancreas cell line | Digestive (and Endocrine) |
| G | 4 | pik15 | + | pancreas cell line | Digestive (and Endocrine) |
| A | 5 | 7F2 | −/− | Mesenchymal Bone Adipocyte cell | Skeletal |
| | | | | line | |
| C | 5 | EI-4 | −/− | T-lymphocyte cell line | |
| E | 5 | P53DN1-5 | −/− | pancreas cell line | Digestive (and Endocrine) |
| G | 5 | pik18 | + | pancreas cell line | Digestive (and Endocrine) |
| A | 6 | CCC4 | −/− | CCC4 | Skeletal |
| C | 6 | C2C12 | −/− | skeletal muscle myoblast | Muscular |
| E | 6 | pid14 | −/− | pancreas cell line | Digestive (and Endocrine) |
| G | 6 | pik31 | +/− | pancreas cell line | Digestive (and Endocrine) |
| A | 7 | OC10A*1 | −/− | bone cell line (pre-bone?) | Skeletal |
| C | 7 | 5FU-17 | +/− | pancreas cell line | Digestive (and Endocrine) |
| E | 7 | pid2 | −/− | pancreas cell line | Digestive (and Endocrine) |
| G | 7 | pik34 | +/− | pancreas cell line | Digestive (and Endocrine) |
| A | 8 | OC10B | ++ | osteoblast cell line | Skeletal |
| E | 8 | pid20 | −/− | pancreas cell line | Digestive (and Endocrine) |
| G | 8 | pik35 | + | pancreas cell line | Digestive (and Endocrine) |
| A | 9 | S194/5.xxo-1 | −/− | Myeloma Cell Line | Lymphatic (and Skeletal) |
| E | 9 | pid21 | −/− | pancreas cell line | Digestive (and Endocrine) |
| A | 10 | P53 hypo #2 | ++ | P53 −/− hypothalamus cell line, | Nervous |
| | | | | growth condition #2 | |
| C | 10 | 5FU-22 | + | pancreas cell line | Digestive (and Endocrine) |
| E | 10 | pid22 | + | pancreas cell line | Digestive (and Endocrine) |
| | | positive control | ++ | | |
| | | negative control | −/− | | |
| | | array 121.02 | | | |
| A | 1 | Paris | ++ | prostate cell line | Male Reproductive |
| C | 1 | GC-1 spg | +++ | testis cell line | Male Reproductive (and |
| | | | | | Endocrine) |
| E | 1 | NB41A3 | +++ | | |
| A | 2 | alpha mem 5/22-3 | +++ | salivary gland cell line | Digestive |
| C | 2 | TM3 | +++ | testis cell line | Male Reproductive (and |
| | | | | | Endocrine) |
| E | 2 | NIH3T3 | +++ | embryonic fibroblast cell line | |
| A | 3 | alpha mem 6/6-1 | +++ | salivary gland cell line | Digestive |
| C | 3 | TM4 | +++ | testis cell line | Male Reproductive (and |
| | | | | | Endocrine) |
| E | 3 | P388D1 | −/− | macrophage cell line | |
| A | 4 | DTH-2m3 | ++ | salivary gland cell line | Digestive |
| C | 4 | R1.1 | −/− | T lymphocyte cell line | Lymphatic (and Endocrine) |
| E | 4 | RAW 264.7 | −/− | monocyte cell line | |
| A | 5 | EGF-2m1 | ++ | salivary gland cell line | Digestive |
| C | 5 | WeHI 7.1 | −/− | thymus lymphoma T-cell line | Lymphatic (and |
| | | | | | Endocrine)Lymphatic (and |
| | | | | | Endocrine) |
| E | 5 | RAW 264.7 +gIFN | −/− | monocyte cell line | |
| A | 6 | EGF-2m3 | ++ | salivary gland cell line | Digestive |
| C | 6 | 3T3 F442A diff. | ++ | 3T3 F442A diff. into adipocytes | |
| E | 6 | STO | ++ | | |
| A | 7 | SAG 5/22-6 | +++ | salivary gland cell line | Digestive |
| C | 7 | 970428 | −/− | 970428 treated day 14 with IL3 and | |
| | | | | stem cell factor | |
| E | 7 | TGFalpha 5H6 | +++ | bone marrow cell line | |
| A | 8 | SAG 5/23-6 | +++ | salivary gland cell line | Digestive |
| C | 8 | ID9 | +++ | | |
| E | 8 | TGFalpha5G1 | +++ | | |
| A | 9 | STD-7m | +++ | salivary gland cell line | Digestive |
| C | 9 | Jaws | + | | |
| A | 10 | A20 | −/− | B lymphocyte reticulum cell sarcoma | Integumentary |
| | | | | cell line | |
| C | 10 | Jaws + gamma interferon | ++ | | |
| | | positive control | ++ | | |
| | | negative control | −/− | | |
| | | library plate | | | |
| A | 1 | Placenta | +/− | | |
| A | 2 | Placenta | −/− | | |
| A | 3 | EB5 | +/− | embroid bodies, day 5 | |
| A | 4 | Testis | +/− | | |
| A | 5 | Ovary | −/− | | |
| A | 6 | Salivary gland | +/− | | |
| A | 7 | Adipocyte | + | | |
| B | 1 | neonatal skin | + | | |
| B | 2 | neonatal skin | +/− | | |
| B | 3 | spleen CD90+ | −/− | | |
| B | 4 | spleen CD90+ | −/− | | |
| | | PMA/Ionomycin stim. | | | |
| B | 5 | MEWT#2 | −/− | | |
| B | 6 | HT-2 | −/− | T-lymphocyte cell line | |
| B | 7 | HT-2, stim. | −/− | T-lymphocyte cell line, stimulated | |
| | | | | with IL4, IL12, gIFN, TNFa, PMA and | |
| | | | | ionomycin | |
| | | positive control | n/a | | |
| | | negative control | −/− | | |

Glucose absorption can be measured in ob/ob mice as follows. Eight female ob/ob mice, approximately 6 weeks old (Jackson Labs, Bar Harbor, Me.) are adapted to a 4 hour daily feeding schedule for two weeks. After two weeks on the feeding schedule, the mice are give 100 μg of separate preparations of the Zsig98 peptides (for example, the polypeptide from amino acid number 25 to amino acid number 85 of SEQ ID NO: 5) in 100 μl sterile 0.1% BSA by oral gavage, immediately after their eating period (post-prandially). Thirty minutes later, the mice are challenged orally with a 0.5 ml volume of 25% glucose. Retro-orbital bleeds are done to determine serum glucose levels. Blood is drawn prior to peptide dosing, prior to oral glucose challenge, and at 1, 2, 4, and 20 hours following the glucose challenge. Post-prandial glucose absorption is measured when peptides are given orally at 100 μg, 30 minutes prior to an oral glucose challenge.

Body weight and glucose clearance can be measured as follows. Sixteen female ob/ob mice, 8 weeks old, (Jackson Labs, Bar Harbor, Me.) are adapted to a special 4 hour daily feeding schedule for two weeks. They are fed ad libitum from 7:30-11:30 am daily. After two weeks on the feeding schedule, the mice are divided into two groups of 8. One group is given 1.0 μg/mouse of a preparation of a Zsig98 polypeptide (for example, the polypeptide as shown in SEQ ID NO: 5 from amino acid 25 to amino acid 85). The other group is given a vehicle (i.e., a scrambled sequence peptide) in 100 μl sterile 0.1% BSQA by oral gavage just prior to receiving food, and at the end of the 4 hour feeding period. The mice are injected twice daily for fourteen days, during which time food intake and body weight is measured daily. On day 14, immediately after the second oral gavage of the SGIP peptides, the mice are challenged orally with an 0.5 ml volume of 25% glucose. Retro-orbital bleeds are done to determine serum glucose levels immediately prior to administration of the Zsig98 polypeptides or vehicle (t=30 min.), and also at 0, 1, 2, and 4 hours following the glucose challenge.

The exocrine or neuroendocrine properties of the Zsig98 polypeptides, agonists, and antagonists of the present invention can be determined by their effect in the AR42J cell line, which is a pancreatic exocrine tumor cell line. (ATCC catalog number CRL-1492™. When stimulated with both activin A and betacellulin, these cells can differentiate into more exocrine-like insulin producing cell. See Mashima, H. et al., J. Clin. Invest. 97:1647-1654, 1996.

The polypeptides, nucleic acid, and/or antibodies of the present invention may be used in treatment of disorders associated with pancreas, diabetes, hypoglycemia; digestive systems including pancreas, colon, and small intestine; neuronal tissues, bone marrow, and peripheral leukocytes; and in disorders associated with glycoprotein synthesis. The molecules of the present invention may used to modulate or to treat or prevent development of pathological conditions in such diverse tissue as pancreas, colon, spinal cord, heart and bone marrow. In particular, certain syndromes or diseases may be amenable to such diagnosis, treatment or prevention.

The Zsig98 polypeptide is expressed in the pancreas. Thus, Zsig98 polypeptide pharmaceutical compositions of the present invention may be useful in prevention or treatment of pancreatic disorders associated with pathological regulation of the expansion of neuroendocrine and exocrine cells in the pancreas, such as IDDM, pancreatic cancer, pathological regulation of blood glucose levels, insulin expression, insulin resistance or digestive function.

As a polypeptide whose expression is over-represented in pancreas islets, the Zsig98 polypeptide of the present invention may act in the neuroendocrine/exocrine cell fate decision pathway and may therefore be capable of regulating the expansion of neuroendocrine and exocrine cells in the pancreas. One such regulatory use is that of islet cell regeneration. Also, it has been hypothesized that the autoimmunity that triggers IDDM starts in utero, and Zsig98 polypeptide is a developmental gene involved in cell partitioning. Assays and animal models are known in the art for monitoring the exocrine/neuroendocrine cell lineage decision, for observing pancreatic cell balance and for evaluating Zsig98 polypeptide, fragment, fusion protein, antibody, agonist or antagonist in the prevention or treatment of the conditions set forth above.

The effect of Zsig98 on insulin and glucagon secretion in the pancreas can be tested using a variety models. Hormone assays, including pancreas perfusion assays, are well known in the art. Basically, the test agent is infused at various fixed concentrations via a cannulated artery and the perfusaate is collected and analyzed fro hormone content. A model for pancreas perfusion is described by Grodsky, G. and Fanska, R., Methods in Enzymology, Vol. 39:364-372 (1975). Briefly, male Sprague-Dawley rats are anesthetized and the pancreas is surgically isolated from surrounding tissues such that the pancreas is connected only at the celiac artery and at the portal vein. The celiac artery is then cannulated and the pancreas is quickly filled with medium. The portal vein is then cannulated, allowing perfusate fractions to be collected. Perfusion is conducted with peristaltic pumps using perfusion medium consisting of oxygenated Krebs-Ringer buffer containing 0.2% BSA, Dextran T-70, and various glucose concentrations. The perfusion is performed at 37° C. in a special chamber. See also Frankel, B. et al., Clin. Res. 21: 273, (1973); and Gerich, J., et al., J. Clin. Endocrinol. Metab. 35:823, (1972).

Increased insulin sensitivity, and beta cell mass in Diabetics is a critical need for many diabetic patients. Novel growth factors, such as Zsig98, that stimulate beta cell function and/or proliferation represent potential therapeutic molecules. Blood glucose homeostasis is controlled by the endocrine cells of the pancreas, located in the islets of Langerhans. Beta cells are the most numerous islet cells. The islet cells monitor the concentration of glucose in the blood, secreting hormones having opposite effects. After a meal the blood glucose concentration increases causing the beta cells to secrete the hormone insulin, thereby reducing blood glucose. Insulin stimulates the uptake of glucose by cells of the body as well as stimulates the conversion of glucose to glycogen in the liver. If the glucose level falls too far, islet alpha cells secrete the hormone glucagon. Glucagon stimulates the breakdown of glycogen to glucose in the liver, increasing blood glucose between meals. Blood glucose levels can be best controlled by minor changes in insulin production and secretion by the pancreatic beta cells and on the capacity of these cells for a large increase of secretion after meals, requiring large stores of insulin. The need for the beta-cell mass to be closely regulated by glucose and hormonal effects on beta-cell replication, size, apoptotic elimination and, under certain conditions, neogenesis from progenitor cells is extremely important. Changes in body mass, pregnancy, insulin sensitivity of peripheral tissues, or tissue injury, if not successfully adapted may lead to the development of chronically elevated blood glucose, or diabetes. The prevalence of diabetes on a global lever has stimulated efforts to develop new therapeutic strategies like beta-cell replacement or regenerative medicine. Existing therapies with exogenous insulin or hypoglycemic agents for type 1 and type 2 diabetes are unsatisfactory, and do not offer a cure and are mostly insufficient for preventing the secondary complications associated with diabetes. See Bouwens, L. et al., Physiol Rev 85:1255-1270, 2005.

Endocrine hormones and growth factors affect the distribution and numbers of specialized cells in endocrine glands that are crucial for survival. Zsig98 is highly expressed from day 11 in mouse embryo to adult. It is predominantly expressed in the adrenals and pancreas, and brain. Like other small, secreted proteins produced specifically by the endocrine glands, GI tract, and brain, zsig98 could be useful in modulating endocrine or exocrine cell activation in these or other gut-brain axis organs. In addition, like other hormones such as insulin, whose primary function is modulation of glucose balance, zsig98 is envisioned as having growth factor effects in the endocrine system. By effecting cells of these organs, Zsig98 may affect diet, metabolism, or production of other endocrine gland hormones and growth factors.

Zsig98 polynucleotides were identified from a mouse lactating gland tissue library. Thus, the Zsig98 polypeptides of the present invention, including agonists, fragments, variants and/or chimeras thereof, can also be used as a supplement to food as well as aids in improving gastrointestinal contractility, improved metabolism, and weight gain. As a protein that may be administered orally, Zsig98, or a combination of agonists, variants, and/or fragments, can be useful as a supplement or adjuvant to a feeding program wherein the mammalian subject suffers from a lack of appetite and/or weight gain. Such conditions are known, for example, as failure to thrive, cachexia, and wasting syndromes. The polypeptides of the present invention may also be useful adapting an infant mammal to digesting more conventional types of food.

As a protein that is expressed in the pancreas and pituitary, the Zsig98 polypeptides, including fragments, will be useful as small peptide growth factors or hormones produced by endocrine tissues, and may be useful in regulating diet, metabolism, or endocrine gland maintenance and function. Assays for measuring this activity include, but are not limited to: 1) Assasys measuring insulin-secretion, such as the INS-1 beta-cell-line and isolated native islets; 2) Assays measuring viability, such as viability of the INS-1 beta-cell-line; 3) Assays measuring insulin receptor phosphorylation, such as on hepatocytes; and 4) Assays measuring insulin action in adipocytes. Commercially available kits that can be used to detect insulin levels in conditioned media, or serum. For example, cells that are capable of producing insulin in response to glucose stimulation, eg. the INS-1 cell line, could be stimulated with zsig98+/−glucose and then the Kit could detect the insulin response. See for example the commercially available kit from Linco Research, Inc., (Human Insulin ELISA Kit, Catalog # EZHI-14K; Linco Research, Inc., St. Charles, Mo.).

Inflammatory reactions cause various clinical manifestations frequently associated with abnormal motility of the gastrointestinal tract, such as nausea, vomiting, ileus or diarrhea. Bacterial lipopolysaccharide (LPS) exposure, for example, induces such an inflammatory condition, which is observed in both humans and experimental animals, and is characterized by biphasic changes in gastrointestinal motility: increased transit in earlier phases and delayed transit in later phases.

Zsig98 polypeptides, such as Zsig98, as well as agonists, fragments, variants and/or chimeras thereof, can also be used to treat gastrointestinal related sepsis. Experimental “sepsis”/endotoxemia is produced in rodents using methods described in Ceregrzyn et al. Neurogastroenterol. Mot. 13:605-613 (2001). These animals develop biphasic alterations in gastrointestinal transit. A Zsig98 polypeptide, such as Zsig98, as well as agonists, fragments, variants and/or chimeras thereof, can be administered (via p.o., i.v., i.p., s.c., or i.m.) at either low (prokinetic) or high (inhibitory) concentrations, depending on the phase of the disease. Gastric emptying and/or intestinal transit would then be measured using one of the models described below.

Morphine and other opioid analgesics are some of the most common pain relievers used, especially following surgery. Because they inhibit the release of acetylcholine from the mesenteric plexus and thereby reduce the propulsive activity in the gastrointestinal tract, individuals taking opioid analgesics often suffer from reduced gastric emptying and intestinal transit. Since Zsig98 simulates intestinal contractility and has gastrointestinal prokinetic activity, Zsig98 may be beneficial in the treatment of opioid-induced motility disorder(s).

The effect of Zsig98 on opioid-induced gastroparesis in experimental rodents can be measured by a well-known model. See Suchitra et al. World J. Gastroenterol. 9:779-783 (2003) and Asai, Arzneim.-Forsch./Drug Res. 48:802-805 (1998). Mice or rats are administered a drug from the opioid class (e.g. morphine) via the appropriate route of administration (p.o., i.p., i.v., s.c., i.m.) at a dose known to inhibit gastric emptying and intestinal transit (e.g. 1-5 mg/kg BW) at a set time prior to administration of the test agent or method used to monitor gastric emptying and intestinal transit (by use of one of the models listed below). The Zsig98 polypeptide, such as Zsig98, as well as agonists, fragments, variants and/or chimeras thereof, is administered at the appropriate time point (via p.o., i.v., i.p., s.c., or i.m.) in relation to the opioid and test meal or method to assess its efficacy in relieving opioid-induced gastroparesis/ileus.

Individuals with neuropathies (e.g. as seen with diabetes) often suffer from gastroparesis and reduced intestinal motility, as a result of a malfunctioning nervous system. Since Zsig98 appears to induce intestinal contractility independently from nervous input, this would suggest that Zsig98 would be beneficial in individuals suffering from neuropathy-induced gastrointestinal disorders.

The effect of Zsig98 on vagotomy-induced gastroparesis in experimental mammals can be measured in an animal model. Thoracic vagotomy is performed in experimental mammals as described, for example, in Takeda et al. Jpn. J. Pharmacol. 81:292-297 (1999) and Hatanaka et al Neurogastroenterol. Motil. 8:227-233 (1996). These animals are characterized by reduced gastric emptying and intestinal transit. The Zsig98 polypeptide, such as Zsig98, as well as agonists, fragments, variants and/or chimeras thereof, is administered (via p.o., i.p., i.v., s.c., or i.m.) as a means to alleviate this vagotomy-induced gastroparesis/ileus, which is monitored using one of the models listed below.

Another indication where Zsig98 can be used to treat gastric dysfunction is gastreoesophageal reflux disease, which is characterized by the backward flow of the stomach contents into the esophagus, often as a result of a reduction in the pressure barrier due to the failure of the lower esophageal sphincter. Prokinetics, such as bethanechol (Urecholine) and metoclopramide (Reglan) have been shown to help strengthen the sphincter and make the stomach empty faster. Metoclopramide also improves muscle action in the digestive tract, but these drugs have frequent side effects that limit their usefulness. Thus a biologic prokinetic, such as a Zsig98 polypeptide, including Zsig98, as well as agonists, fragments, variants and/or chimeras thereof, that improves contraction in the stomach and gastrointestinal tract, with or without improved stability of the esophageal sphincter will be useful to treat gastroesophageal reflux disease.

Methods to investigate effects of atropine in vivo in experimental rodents are well known in the art. See Chadhuri et al. Life Sciences 66:847-854 (2000) and Kaneko et al. Digest. Dis. Sci. 40:2043-2051 (1995). Mice or rats are administered atropine via the appropriate route of administration (p.o., i.p., i.v., s.c., i.m.) at a dose known to inhibit gastric emptying and intestinal transit (e.g. 0.1-2.0 mg/kg BW) at a set time prior to administration of the test agent or method used to monitor gastric emptying and intestinal transit (by use of one of the models listed below). Zsig98 polypeptides, such as Zsig98, as well as agonists, fragments, variants and/or chimeras thereof, is administered at the appropriate time point (via p.o., i.v., i.p., s.c., or i.m.) in relation to the atropine and test meal or method to assess its efficacy in the presence of atropine.

Additional indications where Zsig98 polypeptides, such as Zsig98, as well as agonists, fragments, variants and/or chimeras thereof, can be used to treat gastric dysfunction are gastroparesis, a paralysis of the stomach brought about by a motor abnormality in the stomach or as a complication of diseases such as diabetes, progressive systemic sclerosis, anorexia nervosa or myotonic dystrophy. Diabetic gastroparesis results in delayed gastric emptying, followed by post-prandial distention and vomiting, which can result in poor glycemic control. It is often associated with loss of interstitial cells of Cajal (ICC). Zsig98 polypeptides, such as Zsig98, as well as agonists, fragments, variants and/or chimeras thereof, can also be used to treat gastric dysfunction observed or associated with chronic constipation which can be characterized by intestinal hypomotility, often due to lack of intestinal muscle tone or intestinal spasticity. Another indication where Zsig98 polypeptides, such as Zsig98, as well as agonists, fragments, variants and/or chimeras thereof, can be used to treat gastric dysfunction is dyspepsia, which is defined as an impairment of the power or function of digestion. It can be a symptom of a primary gastrointestinal dysfunction, or a complication of appendicitis, gall bladder disease or malnutrition. Zsig98 polypeptides, such as Zsig98, as well as agonists, fragments, variants and/or chimeras thereof, can also be used to treat gastric dysfunction from emesis which is characterized by symptoms of nausea and vomiting, induced spontaneously, as a result of delayed gastric emptying, or associated with emetogenic cancer chemotherapy or irradiation therapy. In still another indication where Zsig98 polypeptides, such as Zsig98, as well as agonists, fragments, variants and/or chimeras thereof, can be used to treat gastric dysfunction associated with paralytic gastroparesis. This is a paralysis of the stomach brought about by a motor abnormality in the stomach or as a complication of diseases (other than diabetes) such as progressive systemic sclerosis, anorexia nervosa or myotonic dystrophy. It results in delayed gastric emptying, followed by post-prandial distention and vomiting.

For disorders related to deficient gastrointestinal function, clinical signs of improved function include, but are not limited to, increased intestinal transit, increased gastric emptying, flatus, and borborygmi, ability to consume liquids and solids, and/or a reduction in nausea and/or emesis.

Additionally, since Zsig98 reduce contractility when administered at high doses, Zsig98 polypeptides, such as Zsig98, as well as agonists, fragments, variants and/or chimeras thereof, can be used to reduce or inhibit contractility when such effect is desired. This effect may be desired as a solo therapy to treat, for example, diarrhea, including chronic diarrhea and traveler's diarrhea.

For disorders related to hyperactive gastrointestinal contractility, clinincal signs of improved gastrointestinal function include, but are not limited to, slowed gastric emptying, slowed intestinal transit, and/or a reduction in cramps associated with diarrhea.

Zsig98 polypeptides, such as Zsig98, as well as agonists, fragments, variants and/or chimeras thereof, can be used to stimulate chemokine production. Chemokines are small pro-inflammatory proteins that have a broad range of activities involved in the recruitment and function of leukocytes. Rat CINC-1, murine KC, and human GROα are members of the CXC subfamily of chemokines. Chemokines, in general, can be divided into groups that are chemotactic predominatly for neutrophils, and also have angiogenic activity, and those that primarily attract T lymphocytes and monocytes. See Banks, C. et al, J. Pathology 199: 28-35, 2002. Chemokines in the first group display an ELR (Glu-Leu-Arg) amino acid motif at the NH₂ terminus. GROα, for example, contains this motif. GROα also has mitogenic and angiogenic properties and is involved in wound healing and blood vessel formation. (See, for example, Li and Thornhill, Cytokine 12:1409 (2000)). Zsig98 polypeptides, such as Zsig98, as well as agonists, fragments, variants and/or chimeras thereof, can be used to stimulate the production chemokines in vivo. The chemokines can be purified from culture media and used in research or clinical settings. Zsig98 variants can also be identified by the ability to stimulate production of chemokines in vitro or in vivo.

Upregulated chemokine expression correlates with increasing activity of IBD. See Banks, C. et al, J. Pathology 199: 28-35, 2002. Chemokines are able to attract inflammatory cells and are involved in their activation. Similarly, MIP-2 expression has been found to be associated with neutrophil influx in various inflammatory conditions. Zsig98, as well as agonists, fragments, variants and/or chimeras thereof, may be useful in treating Inflammatory Bowel Disease by reducing, inhibiting or preventing chemokine influx in the intestinal tract.

As a protein that can stimulate the production of chemokines, Zsig98 polypeptides, such as Zsig98, as well as agonists, fragments, variants and/or chimeras thereof, may be useful in treating infections, including fungal, bacterial, viral and parasitic infections. Thus, the administration of a Zsig98 polypeptide, such as Zsig98, as well as an agonist, fragment, variant and/or a chimera thereof, may be used as an immune booster to a specific tissue site. For example, Zsig98 administered to gastrointestinal tissue, or to lung tissue, may be useful alone, or in combination therapy to treat infections.

### 11. Models Used to Measure Gastric Emptying and Intestinal Transit

As described above, there are a number of in vivo models to measure gastric function. A few of these models are represented below.

Model 1: Method to Measure Rate and Extent of Gastric Emptying and Intestinal Transit Using Phenol Red/Methyl Cellulose in Experimental Mammals

Fasted animals are given Zsig98 (or other Zsig98 agent, Zsig98 polypeptides, such as Zsig98, as well as agonists, fragments, variants and/or chimeras thereof) by the appropriate route (p.o., i.p., i.v., s.c., i.m.). At the appropriate time point, a non-nutritive semi-solid meal consisting of methylcellulose and phenol red is administered by gavage, and animals are sacrificed at a set time following this meal administration. Transit is assessed by the recovery and spectrophotometric determination of phenol red from designated regions along the gastrointestinal tract. The period of dye recovery in the gastrointestinal tract may be from 10 to 180 minutes, depending on the indication and intestinal site of interest. This model has been used extensively to evaluate the efficacy of other prokinetic drugs on gastric emptying and/or intestinal transit.

In one model measuring gastric emptying, mice receive an intraperitoneal injection of approximately 200 μg of Zsig98 (10 μg/g body weight) or vehicle control followed by 7.5 mg phenol red. Gastric function is measured by monitoring phenol red transport through the gut after twenty minutes. The general behavior of Zsig98 treated animals is observed and compare to the behavior of the control animals.

In another model, male Hartley Guinea pigs at six weeks of age weighing approximately 0.5 kg are euthanized by carbon monoxide. Intestinal tissue is harvested as follows: 2-3 cm longitudinal sections of ileum 10 cm rostral of the cecum, and 2-3 cm longitudinal sections of duodenum, jejunum, and proximal and distal colon.

Tissue is washed in Krebs Ringer's Bicarbonate buffer containing 118.2 mM NaCl, 4.6 mM KCl, 1.2 mm MgS0₄, 24.8 mM NaHC0₃, 1.2 mM KH₂P0₄, 2.5 mM CaCl₂ and 10 mM glucose. Following a thorough wash, the tissue is mounted longitudinally in a Radnoti organ bath perfusion system (SDR Clinical Technology, Sydney Australia) containing oxygenated Krebs buffer warmed and maintained at 37° C. A one gram pre-load is applied and the tissue strips are allowed to incubate for approximately 30 minutes. Baseline contractions are then obtained. Isometric contractions are measured with a force displacement transducer and recorded on a chart recorder using Po-ne-mah Physiology Platform Software. The neurotransmitter 5 Hydroxytryptophane (5HT) (Sigma) at 130 μm, and atropine at 5-10 mM are used as controls. Atropine blocks the muscarinic effect of acetylcholine.

Varying doses of Zsig98 from 1-400 ng/ml are tested for activity on strips of ileum. Muscle contractions are detected immediately after adding Zsig98 protein and are recorded at concentrations as low as 1 ng/ml or 100 picomolar. The EC 50 of this response is measured. Zsig98 is tested for activity in the presence of 5HT, and a secondary contraction is observed. Zsig98 is tested for activity in the presence of 0.1 μM tetrodotoxin (TTX), the nerve action potential antagonist. Zsig98 is also tested for activity in the presence of 100 nM Verapamil, the L-type calcium channel blocker.

In another model, eight-week old female C57Bl/6 mice are fed a test meal consisting of a methylcellulose solution or a control, and both gastric emptying and intestinal transit is measured by determining the amount of phenol red recovered in different sections of the intestine. The test meal consists of a 1.5% aqueous methylcellulose solution containing a non-absorbable dye, 0.05% phenol red (50 mg/100 ml Sigma Chemical Company Catalogue # P4758). Medium viscosity carboxy methylcellulose from Sigma (Catalogue #C4888) with a final viscosity of 400-800 centipoises is used. One group of animals is sacrificed immediately following administration of test meal. These animals represent the standard group, 100% phenol red in stomach or Group VIII. The remaining animals are sacrificed 20 minutes post administration of test meal. Following sacrifice, the stomach is removed and the small intestine is sectioned into proximal, mid and distal gut sections. All tissues are solubilized in 10 mls of 0.1 N NaOH using a tissue homogenizer. Spectrophotometric analysis is used to determine the OD and hence the level of gastric emptying and gut transit.

Each treatment group consists of 10 animals, except for the animals being used as a standard group and the caerulein control group where the n=5. The study is broken down into two days, such that one half of all treatment groups are done on two consecutive days. The animals are fasted for 18 hrs in elevated cages, allowing access to water. The average weight of the mice is measured.

Zsig98 protein with a C-terminal Glu-Glu tag formulated in 20 mM MES buffer, 20 mM NaCl, pH 6.5 is diluted into 0.9% NaCl+0.1% BSA using siliconized tubes. (Sigma sodium chloride solution 0.9%, and Sigma BSA 30% sterile TC tested solution, Sigma Chemical Co, St Louis, Mo.). The protein concentration is adjusted so as to be contained in a 0.2 ml volume per mouse. Vehicle animals receive an equivalent dose of Zsig98 formulation buffer based on the highest (775 ng/g) treatment group.

Treatments are administered in a 0.2 ml volume via IP (intraperitoneal) injection two minutes prior to receiving 0.15 ml phenol red test meal as an oral gavage. Twenty minutes post administration of phenol red, animals are euthanized and stomach and intestinal segments removed. The intestine is measured and divided into three equal segments: proximal, mid and distal gut. The amount of phenol red in each sample is determined by spectrophotometric analysis and expressed as the percent of total phenol red in the stomach (Group VIII). These values are used to determine the amount of gastric emptying and gut transit per tissue collected. The CCK analogue caerulein at 40 ng/gram is used as a positive control and was administered five minutes prior to gavage, at which concentration it inhibits gastric emptying. Colormetric analysis of phenol red recovered from each gut segment and stomach is performed as follows. After euthanization, the stomach and intestinal segments are placed into 10 mls of 0.1 N NaOH and homogenized using a polytron tissue homogenizer. The homogenate is incubated for 1 hour at room temperature then pelleted by centrifugation on a table top centrifuge at 150×g for 20 minutes at 4 degrees C. Proteins are precipitated from 5.0 mls of the homogenate by the addition of 0.5 ml of 20% trichloracetic acid. Following centrifugation, 4 mls of supernatant is added to 4 mls of 0.5 N NaOH. A 200 μl sample was read at 560 nm using Molecular Devices Spectra Max 190 spectrophotometer. The amount of gastric emptying is calculated using the following formula: percent gastric emptying=(1−amount phenol red recovered from test stomach/average amount of phenol red recovered from Group VII stomach)×100. The amount of gastric transit is expressed as the percent of total phenol red recovered.

Model 2: Method to Measure Rate and Extent of Intestinal Transit Using Arabic Gum/Charcoal Meal in Experimental Mammals

Fasted animals would be given Zsig98 (or other Zsig98 agent, Zsig98 polypeptides, such as Zsig98, as well as agonists, fragments, variants and/or chimeras thereof) by the appropriate route (i.p., i.v., s.c., i.m., p.o.). At the appropriate time point, a semi-solid meal consisting of gum arabic and charcoal is administered by gavage, and animals are sacrificed at a set time following this meal administration (Puig and Pol. J. Pharmacol. Experiment. Therap. 287:1068 (1998)). Transit is assessed by the distance that the charcoal meal traveled as a fraction of the total distance of the intestine. The period of transit measurement in the gastrointestinal tract may be from 10 to 180 minutes, depending on the indication and intestinal site of interest. This model has been used extensively to evaluate the efficacy of prokinetic drugs on intestinal transit.

Model 3: Method to Measure Rate and Extent of Gastric Emptying Using Polystyrene Beads (Undigestible Solids) in Experimental Rodents

Gastric emptying is evaluated by determining the emptying of polystyrene beads of a specific diameter (e.g. 1 mm for rats) from the stomach of fasted (24 h) male or female experimental rodents in response to Zsig98 (or another Zsig98 agent, Zsig98 polypeptides, such as Zsig98, as well as agonists, fragments, variants and/or chimeras thereof) via p.o., i.p., s.c., i.v. or i.m. route of administration. Polystyrene beads are administered by gavage and assessed for emptying as previously described (Takeuchi et al. Digest. Dis. Sci. 42; 251-258 (1997)). Animals are sacrificed at a specified time after pellet administration (e.g. 20-180 min), and the stomachs are removed. The number of the pellets remaining in the stomach are counted. In control studies, 90% of pellets would be expected to remain in the stomach after 30 min, and fewer than 10% in the stomach after 3 h. This model has been used extensively to evaluate the gastric emptying efficacy of prokinetic drugs in experimental rodents.

Model 4: Method to Measure Rate and Extent of Gastric Emptying of a Liquid or Solid Test Meal in Experimental Mammals Using Acetaminophen as the Tracer

Fasted animals are given a liquid or solid test meal containing acetominophen as the tracer. The test compound (e.g. Zsig98) is administered p.o., i.v., i.p., s.c., or i.m. either before or after test meal administration. Blood samples are obtained at intervals between 0 and 120 min, and the plasma concentration of acetaminophen (which is a measure of gastric emptying) is measured by HPLC. This is described, for example, in Trudel et al Peptides 24:531-534 (2003).

Model 5: Method to Measure Gastric Emptying of a Solid Meal in Experimental Rodents

Mice or rats (“rodents”) are separated into four groups (Zsig98-; positive control—[erythromycin, metoclopramide, or cisapride]; negative control—[caerulein]; and vehicle-treated groups). Each group contains approximately 10 animals. They are deprived of food for 24 hours, but have free access to water during fast period. Animals are housed one per cage, with floor grids placed in the cages to prevent contact with the bedding or feces. The fasted animals are treated with one of the above agents via one of the following routes of administrations: oral; i.p., i.v., s.c., or i.m.). Animals are introduced to pre-weighed Purina chow individually for a set period of time (e.g. 1 hr) in their home cages (with bedding removed) at the appropriate time point following or prior to administration of test agent. At the end of the feeding period, animals are housed in their home cages without food and water for an additional set period of time. They are then euthanized, the abdominal cavity opened, and stomach removed after clamping the pylorus and cardia. The stomach is weighed, opened, and washed of the gastric content by tap water. The gastric wall is wiped dry, and the empty stomach is weighed again. Gastric contents are collected, dried, and weighed. The amount of food contained in the stomach (as measured in grams) is calculated as the difference between the total weight of the stomach with content and the weight of the stomach wall after the contents are removed. The weight of the pellet and spill in the cage is also measured at the end of the feeding period. The solid food ingested by the animals is determined by the difference between the weight of the Purina chow before feeding and the weight of the pellet and spill at the end of the feeding period. The gastric emptying for the designated period is calculated according to the equation: % of gastric emptying=(1−gastric content/food intake)×100. This model has been used extensively in the literature to assess gastric emptying of a solid meal (Martinez et al. J. Pharmacol. Experiment. Ther. 301: 611-617 (2002)).

Model 6: Method to Measure Rate and Extent of Gastric Emptying of a Solid Test Meal in Experimental Mammals

Fasted animals receive barium sulfate spheroids with a standard meal, followed by administration of the test compound such as Zsig98 polypeptides, such as Zsig98, as well as agonists, fragments, variants and/or chimeras thereof (via p.o., i.v., i.p., s.c., or i.m) either before or after the meal. Gastric emptying is measured by means of X-ray location, with passage being monitored at least every 15 min-2 h. This method is described, for example, in Takeda et al. Jpn. J. Pharmacol. 81:292-297 (1999).

Model 7: Method to Measure Colonic Propulsive Motility in Experimental Rodents.

This is used to demonstrate and characterize the pharmacological effects of compounds on colonic propulsive motility in experimental rodents as described (Martinez et al. J. Pharmacol. Experiment. Ther. 301:611-617 (2002)). The test is based on the reflex expulsion of a glass bead from the distal colon, which is indicative of drug effects on the reflex arc. This test is useful in evaluating whether diarrhea is a side effect. Mice or rats are fasted for one hour prior to administrations of the test compound, such Zsig98 polypeptides, such as Zsig98, as well as agonists, fragments, variants and/or chimeras thereof, or vehicle by the appropriate route (i.p., s.c., i.m., p.o., i.v.), followed 30 minutes (or other appropriate time) later by the insertion of a glass bead into the distal colon. Rodents are marked for identification and placed in large glass beakers (or other) for observation. The time required for expulsion of the bead is noted for each rodent.

Model 8: Model to Measure Gastrointestinal Motor Activity in Dogs.

Dogs are anesthetized and the abdominal cavity opened. Extraluminal force transducers (sensor to measure contraction) are sutured onto five (5) sites, i.e., the gastric antrum, 3 cm proximal to the pyloric ring, the duodenum, 5 cm distal to the pyloric ring, the jejunum, 70 cm distal to the pyloric ring, the ileum, 5 cm proximal to the ileum-colon junction, and the colon, 5 cm distal to the ileum-colon junction. The lead wires of these force transducers are taken out of the abdominal cavity and then brought out through a skin incision made between the scapulae, at which a connector is connected. After the operation, a jacket protector is placed on the dog to protect the connector. Measurement of the gastrointestinal motor activity is started two weeks after the operation. For ad libitum measurement, a telemeter (electrowave data transmitter) is connected with the connector to determine the contractive motility at each site of the gastrointestinal tract. The data is stored in a computer via a telemeter for analysis. A test compound, such as Zsig98 polypeptides, such as Zsig98, as well as agonists, fragments, variants and/or chimeras thereof, is administered via the appropriate route (p.o., i.v., i.p., s.c., i.m.) at the appropriate time point to assess its ability to affect gastrointestinal motor activity. This can be performed in normal dogs or dogs in which gastroparesis/ileus has been induced. The above method is a modification of those in Yoshida. and Ito. J. Pharmacol. Experiment. Therap. 257, 781-787 (1991) and Furuta et al. Biol. Pharm. Bull. 25:103-1071 (2002).

### 12. Additional Methods to Measure Gastric Function

Model of Pain Assessment Associated with Gut Distention (in Rats; Rabbits; Dogs)

Indication: Inflammatory Bowel Disease (IBD), Irritable Bowl Syndrome (IBS), gastroparesis, ileus, dyspepsia.

Animals are surgically prepared with electrodes implanted on the proximal colon and striated muscles, and catheters implanted in lateral ventricles of the brain. Rectal distension is performed by inflation of a balloon rectally inserted, and the pressure eliciting a characteristic visceromotor response is measured. A test compound, such as Zsig98 polypeptides, such as Zsig98, as well as agonists, fragments, variants and/or chimeras thereof, is administered via the appropriate route (p.o., i.p., s.c., i.v., or i.m.) and at the appropriate time (i.e. ˜20 min, if i.p. or i.c.v.) prior to distention. Test compound is evaluated for its ability to affect colonic motility, abdominal contractions, and visceral pain.

Model to Assess Emesis (in Ferrets).

Indication: emesis (primary or as a result of gastroparesis)

The anti-emetic activity of a test compound is tested by its ability to inhibit cisplatin- or syrup of ipecac-induced emesis in the ferret (since mice and rats can not vomit). In this model the onset of retching and vomiting occurs approximately 1 h after the administration of cisplatin (200 mg/m.sup.2 i.p.). At the first retch in response to cisplatin, the test compound, Zsig98 polypeptides, such as Zsig98, as well as agonists, fragments, variants and/or chimeras thereof, is administered (e.g. i.p., p.o., i.v., s.c., i.c.v.) and its effect on emesis determined by comparison with appropriate controls (e.g. water). If using ipecac to induce emesis, the test compound may be given at appropriate time points prior to the ipecac. Latency to the first retch, the first vomit and the number of retching and vomiting episodes are recorded over 60 min. Data are expressed as the mean latency (in min) to first retch or vomit; the mean number of emetic episodes per ferret based on animals that did not exhibit emesis as well as those that did, and the mean number of retches/vomits exhibited by animals that remained responsive to ipecac (“responders”). Ferrets that fail to exhibit emesis are omitted from the latter calculation.

[i.e. (.+−.) cis-3-(2-methoxybenzylamino)-2-phenyl piperidine exhibited anti-emetic activity when administered at a dose of 3 mg/kg i.p.]

Models of (Interstitial Cells of Cajal) ICC Loss.

Loss of ICC results in serious gastrointestinal motor dysfunction, and is seen in many diseases associated with altered gastrointestinal function. Antibodies to Kit provide the opportunity to evaluate ICC networks in gastrointestinal muscles in motility disorders.

Indications: diabetic gastroparesis; IBD; pseudo-obstruction, chronic constipation.

Inducible Example: BALB/c mouse pups are treated with a monoclonal antibody (ACK2) to Kit for 4 d postnatally This suppresses the development of c-kit, resulting in a severe disorder of gut motility. A test compound, Zsig98 polypeptides, such as Zsig98, as well as agonists, fragments, variants and/or chimeras thereof, is administered to assess its affect on gut motility. Isolated segments of the intestine from the Kit-treated mice may also be tested for rhythmic contraction and relaxation in vitro, in response to the test compound.

Spontaneous Example: The spontaneously diabetic NOD/LtJ mouse (Jackson Labs) develop delayed gastric emptying, impaired electrical pacemaking, and reduced motor neurotransmission. A test compound, Zsig98 polypeptides, such as Zsig98, as well as agonists, fragments, variants and/or chimeras thereof, administered to assess its affect on gastric emptying (i.e. via phenol red/methyl cellulose) and gut motility. Isolated segments of the intestine from these mice may also be tested for rhythmic contraction and relaxation in vitro, in response to the test compound administration.

Furthermore, prolonged gastrointestinal stasis often complicates the course of patients with sepsis (Hemann G, et al., Am. J. Phys. Regul. Integr. Compr. Physiol. 276:R59-R68, 1999). Activation of a systemic immune response by injury, infection, radiation, or chemotherapy, is often accompanied by gastric stasis which is perceived as nausea, loss of appetite and vomiting (Emch G., et al., Am. J. Physiol. Gastrointest. Liver Physiol. 279: G5582-G586, 2000). Thus, Zsig98 and it agonists may be useful in treating sepsis related to gastrointestinal stasis or ileus.

Additionally, such as Zsig98, as well as agonists, fragments, variants and/or chimeras thereof, may be useful in treating patients with nausea and vomiting, especially where the nausea and vomiting are related to, or a result of ileus or other gastrointestinal motility disorders. These include when the vomiting is related to treatment for cancer, such as a prophylaxis, or post-administered, for chemotherapy.

The Zsig98 polypeptides of the present invention, such as Zsig98, as well as agonists, fragments, variants and/or chimeras thereof, can also be used as a supplement to food. Zsig98 polypeptides have been purified from bovine milk. Additionally, increased gastrointestinal contractility can be conducive to improved metabolism and weight gain. As a protein that can be administered orally, Zsig98 or a combination of agonists, variants, and/or fragments, can be useful as a supplement or adjuvant to a feeding program wherein the mammalian subject suffers from a lack of appetite and/or weight gain. Such conditions are known, for example, as failure to thrive, cachexia, and wasting syndromes. The polypeptides of the present invention may also be useful adapting an infant mammal to digesting more conventional types of food.

Generally, the dosage of administered polypeptide, protein or peptide will vary depending upon such factors as the patient's age, weight, height, sex, general medical condition and previous medical history. Typically, it is desirable to provide the recipient with a dosage of a molecule having Zsig98 activity, which is in the range of from about 1 pg/kg to 10 mg/kg (amount of agent/body weight of patient), although a lower or higher dosage also may be administered as circumstances dictate.

Administration of a molecule having Zsig98 activity to a subject can be intravenous, intraarterial, intraperitoneal, intramuscular, subcutaneous, intrapleural, intrathecal, by perfusion through a regional catheter, inhalation, as a suppository, or by direct intralesional injection. When administering therapeutic proteins by injection, the administration may be by continuous infusion or by single or multiple boluses. Alternatively, Zsig98 polypeptides, such as Zsig98, as well as agonists, fragments, variants and/or chimeras thereof, can be administered as a controlled release formulation.

Additional routes of administration include oral, dermal, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising such as Zsig98, as well as agonists, fragments, variants and/or chimeras thereof, can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer such as Zsig98, as well as agonists, fragments, variants and/or chimeras thereof, (Potts et al., Pharm. Biotechnol. 10:213 (1997)).

Zsig98 proteins can also be applied topically as, for example, liposomal preparations, gels, salves, as a component of a glue, prosthesis, or bandage, and the like.

A pharmaceutical composition comprising molecules having Zsig98 activity can be furnished in liquid form, in an aerosol, or in solid form. Proteins having Zsig98 activity can be administered as a conjugate with a pharmaceutically acceptable water-soluble polymer moiety. As an illustration, a Zsig98-polyethylene glycol conjugate is useful to increase the circulating half-life of the interferon, and to reduce the immunogenicity of the polypeptide. Liquid forms, including liposome-encapsulated formulations, are illustrated by injectable solutions and oral suspensions. Exemplary solid forms include capsules, tablets, and controlled-release forms, such as a miniosmotic pump or an implant. Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

A pharmaceutical composition comprising a protein, polypeptide, or peptide having Zsig98 activity can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, molecules having Zsig98 activity and a pharmaceutically acceptable carrier are administered to a patient in a therapeutically effective amount. A combination of a protein, polypeptide, or peptide having Zsig98 activity and a pharmaceutically acceptable carrier is said to be administered in a “therapeutically effective amount” if the amount administered is physiologically significant. An agent is physiologically significant if its presence results in a detectable change in the physiology of a recipient patient.

For example, the present invention includes methods of increasing or decreasing gastrointestinal contractility, gastric emptying and/or intestinal transit, comprising the step of administering a composition comprising a Zsig98 polypeptide, such as Zsig98, as well as agonists, fragments, variants and/or chimeras thereof, to the patient. In an in vivo approach, the composition is a pharmaceutical composition, administered in a therapeutically effective amount to a mammalian subject.

A pharmaceutical composition comprising molecules having Zsig98 activity can be furnished in liquid form, or in solid form. Liquid forms, including liposome-encapsulated formulations, are illustrated by injectable solutions and oral suspensions. Exemplary solid forms include capsules, tablets, and controlled-release forms, such as a miniosmotic pump or an implant. Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

Zsig98 pharmaceutical compositions may be supplied as a kit comprising a container that comprises Zsig98, a Zsig98 agonist, or a Zsig98 antagonist (e.g., an anti-Zsig98 antibody or antibody fragment). For example, Zsig98 can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise written information on indications and usage of the pharmaceutical composition. Moreover, such information may include a statement that the Zsig98 composition is contraindicated in patients with known hypersensitivity to Zsig98.

The effect of Zsig98 on gastric emptying can be measured in mice as follows. The mice receive an intraperitoneal injection of approximately 200 μg of Zsig98 (10 μg/g body weight) or vehicle control followed by 7.5 mg phenol red. Gastric function is measured by monitoring phenol red transport through the gut after twenty minutes. The general behavior of Zsig98 treated animals is observed and compared against the behavior of the control animals. A reduction of gastric transit time is indicative of improved gastric emptying.

In another model, male Hartley Guinea pigs at six weeks of age weighing approximately 0.5 kg are euthanized by carbon monoxide. Intestinal tissue is harvested as follows: 2-3 cm longitudinal sections of ileum 10 cm rostral of the cecum, and 2-3 cm longitudinal sections of duodenum, jejunum, and proximal and distal colon. Tissue is washed in Krebs Ringer's Bicarbonate buffer containing 118.2 mM NaCl, 4.6 mM KCl, 1.2 mm MgS0₄, 24.8 mM NaHC0₃, 1.2 mM KH₂P0₄, 2.5 mM CaCl₂ and 10 mM glucose. Following a thorough wash, the tissue is mounted longitudinally in a Radnoti organ bath perfusion system (SDR Clinical Technology, Sydney Australia) containing oxygenated Krebs buffer warmed and maintained at 37° C. A one gram pre-load is applied and the tissue strips are allowed to incubate for approximately 30 minutes. Baseline contractions are then obtained. Isometric contractions are measured with a force displacement transducer and recorded on a chart recorder using Po-ne-mah Physiology Platform Software. The neurotransmitter 5 Hydroxytryptophane (5HT) (Sigma) at 130 μm, and atropine at 5-10 mM are used as controls. Atropine blocks the muscarinic effect of acetylcholine. Varying doses of Zsig98 from 1-400 ng/ml are tested for activity on strips of ileum. Muscle contractions are detected immediately after adding Zsig98 protein and are recorded at concentrations as low as 1 ng/ml or 100 picomolar. The EC 50 of this response is measured. Zsig98 is tested for activity in the presence of 5HT, and a secondary contraction is observed. Zsig98 is tested for activity in the presence of 0.1 μM tetrodotoxin (TTX), the nerve action potential antagonist. Zsig98 is also tested for activity in the presence of 100 nM Verapamil, the L-type calcium channel blocker. A significant reduction in the amplitude of the contractile response is indicative of gastric contractility.

The effect of Zsig98 on gastrointestinal contractility can also be measured in a guinea pig ileum organ bath assay. Longitudinal strips of guinea pig ileum are mounted in the organ bath and allowed to stabilize for approximately 20 minutes. Acetylcholine (ACH) at a concentration of 10 μg/ml is added to tissue to confirm contractile activity. Two flush and fill cycles are run to wash ACH from the intestinal tissue. Baseline activity is confirmed for approximately 25 minutes. Zsig98 is added to the organ bath at a final concentration of 1.0 ng/ml and the amount of deflection is recorded. The 1.0 ng/ml Zsig98 dose is left on the tissue for 5 minutes to allow the tissue to return to baseline levels, and then a 10 ng/ml dose is added. Contractile response is measured by the deflection. The 10 ng/ml dose is left on for another 5 minutes before dosing the tissue with a 20 ng/ml dose of Zsig98.

Organ bath testing can also be performed with Zsig98 using at a variety of tissues obtained from guinea pigs. A force transducer is used to record the mechanical contraction using IOX software (EMKa technologies, Falls Church, Va.) and Datanalyst software (EMKa technologies, Falls Church, Va.). Tissues can include duodenum, jejunum, ileum, trachea, esophagus, aorta, stomach, gall bladder, bladder and uterus among others. Two month old male guinea pigs (Hartley, Charles River Labs) weighing ˜250 to 300 g are fasted with access to drinking water for ˜18 hours then euthanized by CO₂ asphyxiation. All tissues are rinsed with Krebs buffer (1.2 mM MgSO₄, 115 mM NaCl, 11.5 mM glucose, 23.4 mM NaHCO₃, 4.7 mM KCl, 1.2 mM NaH₂PO₄, and 2.4 mM CaCl₂, oxygenated with 95% O₂-5% CO₂, pH 7.4, temperature 37° C.) then suspended in the 5 ml organ bath and pre-tensioned. All tissues are tested with positive controls to establish their viability prior to running. Positive controls are CCK-8, acetylcholine (ACH), histamine, or 5HT (purchased from Sigma (Saint Louis, Mo.)). All tissues are treated with a vehicle control, phosphate buffered saline (PBS), to rule out the possibility of vehicle effects.

In another model, eight-week old female C57Bl/6 mice are fed a test meal consisting of a methylcellulose solution or a control, and both gastric emptying and intestinal transit is measured by determining the amount of phenol red recovered in different sections of the intestine. The test meal consists of a 1.5% aqueous methylcellulose solution containing a non-absorbable dye, 0.05% phenol red (50 mg/100 ml Sigma Chemical Company Catalogue # P4758). Medium viscosity carboxy methylcellulose from Sigma (Catalogue #C4888) with a final viscosity of 400-800 centipoises is used. One group of animals is sacrificed immediately following administration of test meal. These animals represent the standard group, 100% phenol red in stomach or Group VIII. The remaining animals are sacrificed 20 minutes post administration of test meal. Following sacrifice, the stomach is removed and the small intestine is sectioned into proximal, mid and distal gut sections. All tissues are solubilized in 10 mls of 0.1 N NaOH using a tissue homogenizer. Spectrophotometric analysis is used to determine the OD and hence the level of gastric emptying and gut transit. Each treatment group consists of 10 animals, except for the animals being used as a standard group and the caerulein control group where the n=5. The study is broken down into two days, such that one half of all treatment groups are done on two consecutive days. The animals are fasted for 18 hrs in elevated cages, allowing access to water. The average weight of the mice is measured. Zsig98 protein with a C-terminal Glu-Glu tag formulated in 20 mM MES buffer, 20 mM NaCl, pH 6.5 is diluted into 0.9% NaCl+0.1% BSA using siliconized tubes. (Sigma sodium chloride solution 0.9%, and Sigma BSA 30% sterile TC tested solution, Sigma Chemical Co, St Louis, Mo.). The protein concentration is adjusted so as to be contained in a 0.2 ml volume per mouse. Vehicle animals received an equivalent dose of Zsig98 formulation buffer based on the highest (775 ng/g) treatment group. Treatments are administered in a 0.2 ml volume via IP (intraperitoneal) injection two minutes prior to receiving 0.15 ml phenol red test meal as an oral gavage. Twenty minutes post administration of phenol red, animals are euthanized and stomach and intestinal segments removed. The intestine is measured and divided into three equal segments: proximal, mid and distal gut. The amount of phenol red in each sample is determined by spectrophotometric analysis and expressed as the percent of total phenol red in the stomach (Group VIII). These values are used to determine the amount of gastric emptying and gut transit per tissue collected. The CCK analogue caerulein at 40 ng/gram is used as a positive control and is administered five minutes prior to gavage, at which concentration it inhibits gastric emptying. Colormetric analysis of phenol red recovered from each gut segment and stomach is performed as follows. After euthanization, the stomach and intestinal segments are placed into 10 mls of 0.1 N NaOH and homogenized using a polytron tissue homogenizer. The homogenate is incubated for 1 hour at room temperature then pelleted by centrifugation on a table top centrifuge at 150×g for 20 minutes at 4 degrees C. Proteins are precipitated from 5.0 mls of the homogenate by the addition of 0.5 ml of 20% trichloracetic acid. Following centrifugation, 4 mls of supernatant is added to 4 mls of 0.5 N NaOH. A 200 μl sample is read at 560 nm using Molecular Devices Spectra Max 190 spectrophotometer. The amount of gastric emptying is calculated using the following formula: percent gastric emptying=(1−amount phenol red recovered from test stomach/average amount of phenol red recovered from Group VII stomach)×100. The amount of gastric transit is expressed as the percent of total phenol red recovered.

The activity of Zsig98, it agonists, antagonist, and fragments, can be measured in a reporter assay. Rat2 fibroblast cells (ATCC #CRL-1764, American Type Culture Collection, Manassass, Va.) are transfected with a SRE luciferase reporter construct and selected for stable clones. These are then transfected with constructs for the Zsgi98 receptor. Cells are trypsinized and seeded in Corning 96-well white plates at 3,000 cells/well in media containing 1% serum and incubated overnight at 37° C. and 5% CO₂. Media is removed and samples are added in triplicate to cells in media containing 0.5% BSA and incubated for four hours at 37° C. and 5% C0₂. After media is removed the cells are lysed and luciferase substrate is added according to the Promega luciferase assay system (Promega Corp., Madison, Wis.). All data are reported as fold-induction of the RLU (relative light units) from the luminometer divided by the basal signal (media only).

Polyclonal antibodies can be prepared by immunizing 2 female New Zealand white rabbits with the purified recombinant protein or with fragments thereof such as CSLDARTETLLLQAERRALCACWPAGH. The rabbits are each given an initial intraperitoneal (ip) injection of 200 μg of purified protein in Complete Freund's Adjuvant followed by booster ip injections of 100 μg peptide in Incomplete Freund's Adjuvant every three weeks. Seven to ten days after the administration of the second booster injection (3 total injections), the animals are bled and the serum is collected. The animals are then boosted and bled every three weeks. Polyclonal antibodies are purified from the immunized rabbit serum using a 5 ml. Protein A sepharose column (Pharmacia LKB). Following purification, the polyclonal antibodies are dialyzed with several changes of 20 times the antibody volume of PBS over a time period of at least 8 hours. The antibodies are characterized by ELISA using the purified recombinant protein or fragment as the antibody target. The lower limit of detection (LLD) of the rabbit anti-huZsig98 purified antibody is determined.

The purified polyclonal Zsig98 antibodies are characterized for their ability to bind recombinant human Zsig98 polypeptides using the ORIGEN(®) Immunoassay System (IGEN Inc, Gaithersburg, Md.). In this assay, the antibodies are used to quantitatively determine the level of recombinant huZsig98 in rat serum samples. An immunoassay format is designed that consisted of a biotinylated capture antibody and a detector antibody, which is labeled with ruthenium (II) tris-bipyridal chelate, thereby sandwiching the antigen in solution and forming an immunocomplex. Streptavidin-coated paramagnetic beads are then bound to the immunocomplex. In the presence of tripropylamine, the ruthenylated Ab gives off light, which is measured by the ORIGEN analyzer. Concentration curves of 0.1-50 ng/ml huZsig98 made quantitation possible using 50 microliters of sample.

### 13. Therapeutic Uses of Zsig98 Nucleotide Sequences

The present invention includes the use of Zsig98 nucleotide sequences to provide Zsig98 amino acid sequences to a subject in need of proteins, polypeptides, or peptides having Zsig98 activity, as discussed in the previous section. In addition, a therapeutic expression vector can be provided that inhibits Zsig98 gene expression, such as an anti-sense molecule, a ribozyme, or an external guide sequence molecule.

There are numerous approaches to introduce a Zsig98 gene to a subject, including the use of recombinant host cells that express Zsig98, delivery of naked nucleic acid encoding Zsig98, use of a cationic lipid carrier with a nucleic acid molecule that encodes Zsig98, and the use of viruses that express Zsig98, such as recombinant retroviruses, recombinant adeno-associated viruses, recombinant adenoviruses, and recombinant Herpes simplex viruses [HSV] (see, for example, Mulligan, Science 260:926 (1993), Rosenberg et al., Science 242:1575 (1988), LaSalle et al., Science 259:988 (1993), Wolff et al., Science 247:1465 (1990), Breakfield and Deluca, The New Biologist 3:203 (1991)). In an ex vivo approach, for example, cells are isolated from a subject, transfected with a vector that expresses a Zsig98 gene, and then transplanted into the subject.

In order to effect expression of a Zsig98 gene, an expression vector is constructed in which a nucleotide sequence encoding a Zsig98 gene is operably linked to a core promoter, and optionally a regulatory element, to control gene transcription. The general requirements of an expression vector are described above.

Alternatively, a Zsig98 gene can be delivered using recombinant viral vectors, including for example, adenoviral vectors (e.g., Kass-Eisler et al., Proc. Nat'l Acad. Sci. USA 90:11498 (1993), Kolls et al., Proc. Nat'l Acad. Sci. USA 91:215 (1994), Li et al., Hum. Gene Ther. 4:403 (1993), Vincent et al., Nat. Genet. 5:130 (1993), and Zabner et al., Cell 75:207 (1993)), adenovirus-associated viral vectors (Flotte et al., Proc. Nat'l Acad. Sci. USA 90:10613 (1993)), alphaviruses such as Semliki Forest Virus and Sindbis Virus (Hertz and Huang, J. Vir. 66:857 (1992), Raju and Huang, J. Vir. 65:2501 (1991), and Xiong et al., Science 243:1188 (1989)), herpes viral vectors (e.g., U.S. Pat. Nos. 4,769,331, 4,859,587, 5,288,641 and 5,328,688), parvovirus vectors (Koering et al., Hum. Gene Therap. 5:457 (1994)), pox virus vectors (Ozaki et al., Biochem. Biophys. Res. Comm. 193:653 (1993), Panicali and Paoletti, Proc. Nat'l Acad. Sci. USA 79:4927 (1982)), pox viruses, such as canary pox virus or vaccinia virus (Fisher-Hoch et al., Proc. Nat'l Acad. Sci. USA 86:317 (1989), and Flexner et al., Ann. N.Y. Acad. Sci. 569:86 (1989)), and retroviruses (e.g., Baba et al., J. Neurosurg 79:729 (1993), Ram et al., Cancer Res. 53:83 (1993), Takamiya et al., J. Neurosci. Res 33:493 (1992), Vile and Hart, Cancer Res. 53:962 (1993), Vile and Hart, Cancer Res. 53:3860 (1993), and Anderson et al., U.S. Pat. No. 5,399,346). Within various embodiments, either the viral vector itself, or a viral particle which contains the viral vector may be utilized in the methods and compositions described below.

As an illustration of one system, adenovirus, a double-stranded DNA virus, is a well-characterized gene transfer vector for delivery of a heterologous nucleic acid molecule (for a review, see Becker et al., Meth. Cell Biol. 43:161 (1994); Douglas and Curiel, Science & Medicine 4:44 (1997)). The adenovirus system offers several advantages including: (i) the ability to accommodate relatively large DNA inserts, (ii) the ability to be grown to high-titer, (iii) the ability to infect a broad range of mammalian cell types, and (iv) the ability to be used with many different promoters including ubiquitous, tissue specific, and regulatable promoters. In addition, adenoviruses can be administered by intravenous injection, because the viruses are stable in the bloodstream.

Using adenovirus vectors where portions of the adenovirus genome are deleted, inserts are incorporated into the viral DNA by direct ligation or by homologous recombination with a co-transfected plasmid. In an exemplary system, the essential E1 gene is deleted from the viral vector, and the virus will not replicate unless the E1 gene is provided by the host cell. When intravenously administered to intact animals, adenovirus primarily targets the liver. Although an adenoviral delivery system with an E1 gene deletion cannot replicate in the host cells, the host's tissue will express and process an encoded heterologous protein. Host cells will also secrete the heterologous protein if the corresponding gene includes a secretory signal sequence. Secreted proteins will enter the circulation from tissue that expresses the heterologous gene (e.g., the highly vascularized liver).

Moreover, adenoviral vectors containing various deletions of viral genes can be used to reduce or eliminate immune responses to the vector. Such adenoviruses are E1-deleted, and in addition, contain deletions of E2A or E4 (Lusky et al., J. Virol. 72:2022 (1998); Raper et al., Human Gene Therapy 9:671 (1998)). The deletion of E2b has also been reported to reduce immune responses (Amalfitano et al., J. Virol. 72:926 (1998)). By deleting the entire adenovirus genome, very large inserts of heterologous DNA can be accommodated. Generation of so called “gutless” adenoviruses, where all viral genes are deleted, are particularly advantageous for insertion of large inserts of heterologous DNA (for a review, see Yeh. and Perricaudet, FASEB J. 11:615 (1997)).

High titer stocks of recombinant viruses capable of expressing a therapeutic gene can be obtained from infected mammalian cells using standard methods. For example, recombinant HSV can be prepared in Vero cells, as described by Brandt et al., J. Gen. Virol. 72:2043 (1991), Herold et al., J. Gen. Virol. 75:1211 (1994), Visalli and Brandt, Virology 185:419 (1991), Grau et al., Invest. Ophthalmol. Vis. Sci. 30:2474 (1989), Brandt et al., J. Virol. Meth. 36:209 (1992), and by Brown and MacLean (eds.), HSV Virus Protocols (Humana Press 1997).

Alternatively, an expression vector comprising a Zsig98 gene can be introduced into a subject's cells by lipofection in vivo using liposomes. Synthetic cationic lipids can be used to prepare liposomes for in vivo transfection of a gene encoding a marker (Felgner et al., Proc. Nat'l Acad. Sci. USA 84:7413 (1987); Mackey et al., Proc. Nat'l Acad. Sci. USA 85:8027 (1988)). The use of lipofection to introduce exogenous genes into specific organs in vivo has certain practical advantages. Liposomes can be used to direct transfection to particular cell types, which is particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Electroporation is another alternative mode of administration of Zsig98 nucleic acid molecules. For example, Aihara and Miyazaki, Nature Biotechnology 16:867 (1998), have demonstrated the use of in vivo electroporation for gene transfer into muscle.

In an alternative approach to gene therapy, a therapeutic gene may encode a Zsig98 anti-sense RNA that inhibits the expression of Zsig98. Suitable sequences for Zsig98 anti-sense molecules can be derived from the nucleotide sequences of Zsig98 disclosed herein.

Alternatively, an expression vector can be constructed in which a regulatory element is operably linked to a nucleotide sequence that encodes a ribozyme. Ribozymes can be designed to express endonuclease activity that is directed to a certain target sequence in a mRNA molecule (see, for example, Draper and Macejak, U.S. Pat. No. 5,496,698, McSwiggen, U.S. Pat. No. 5,525,468, Chowrira and McSwiggen, U.S. Pat. No. 5,631,359, and Robertson and Goldberg, U.S. Pat. No. 5,225,337). In the context of the present invention, ribozymes include nucleotide sequences that bind with Zsig98 mRNA.

In another approach, expression vectors can be constructed in which a regulatory element directs the production of RNA transcripts capable of promoting RNase P-mediated cleavage of mRNA molecules that encode a Zsig98 gene. According to this approach, an external guide sequence can be constructed for directing the endogenous ribozyme, RNase P, to a particular species of intracellular mRNA, which is subsequently cleaved by the cellular ribozyme (see, for example, Altman et al., U.S. Pat. No. 5,168,053, Yuan et al., Science 263:1269 (1994), Pace et al., international publication No. WO 96/18733, George et al., international publication No. WO 96/21731, and Werner et al., international publication No. WO 97/33991). Preferably, the external guide sequence comprises a ten to fifteen nucleotide sequence complementary to Zsig98 mRNA, and a 3′-NCCA nucleotide sequence, wherein N is preferably a purine. The external guide sequence transcripts bind to the targeted mRNA species by the formation of base pairs between the mRNA and the complementary external guide sequences, thus promoting cleavage of mRNA by RNase P at the nucleotide located at the 5′-side of the base-paired region.

In general, the dosage of a composition comprising a therapeutic vector having a Zsig98 nucleotide acid sequence, such as a recombinant virus, will vary depending upon such factors as the subject's age, weight, height, sex, general medical condition and previous medical history. Suitable routes of administration of therapeutic vectors include intravenous injection, intraarterial injection, intraperitoneal injection, intramuscular injection, intratumoral injection, and injection into a cavity that contains a tumor.

A composition comprising viral vectors, non-viral vectors, or a combination of viral and non-viral vectors of the present invention can be formulated according to known methods to prepare pharmaceutically useful compositions, whereby vectors or viruses are combined in a mixture with a pharmaceutically acceptable carrier. As noted above, a composition, such as phosphate-buffered saline is said to be a “pharmaceutically acceptable carrier” if its administration can be tolerated by a recipient subject. Other suitable carriers are well-known to those in the art (see, for example, Remington's Pharmaceutical Sciences, 19th Ed. (Mack Publishing Co. 1995), and Gilman's the Pharmacological Basis of Therapeutics, 7th Ed. (MacMillan Publishing Co. 1985)).

For purposes of therapy, a therapeutic gene expression vector, or a recombinant virus comprising such a vector, and a pharmaceutically acceptable carrier are administered to a subject in a therapeutically effective amount. A combination of an expression vector (or virus) and a pharmaceutically acceptable carrier is said to be administered in a “therapeutically effective amount” if the amount administered is physiologically significant. An agent is physiologically significant if its presence results in a detectable change in the physiology of a recipient subject.

When the subject treated with a therapeutic gene expression vector or a recombinant virus is a human, then the therapy is preferably somatic cell gene therapy. That is, the preferred treatment of a human with a therapeutic gene expression vector or a recombinant virus does not entail introducing into cells a nucleic acid molecule that can form part of a human germ line and be passed onto successive generations (i.e., human germ line gene therapy).

### 14. Detection of Zsig98 Gene Expression with Nucleic Acid Probes

Nucleic acid molecules can be used to detect the expression of a Zsig98 gene in a biological sample. Such probe molecules include double-stranded nucleic acid molecules comprising the nucleotide sequence of SEQ ID NO:1, or a fragment thereof, as well as single-stranded nucleic acid molecules having the complement of the nucleotide sequence of SEQ ID NO:1, or a fragment thereof. Probe molecules may be DNA, RNA, oligonucleotides, and the like.

Illustrative probes comprise a portion of the nucleotide sequence of nucleotides 66 to 161 of SEQ ID NO:1, the nucleotide sequence of nucleotides 288 to 389 of SEQ ID NO:1, or the complement of such nucleotide sequences. An additional example of a suitable probe is a probe consisting of nucleotides 354 to 382 of SEQ ID NO:1, or a portion thereof. As used herein, the term “portion” refers to at least eight nucleotides to at least 20 or more nucleotides.

For example, nucleic acid molecules comprising a portion of the nucleotide sequence of SEQ ID NO:1 can be used to detect activated neutrophils. Such molecules can also be used to identity therapeutic or prophylactic agents that modulate the response of a neutrophil to a pathogen.

In a basic detection assay, a single-stranded probe molecule is incubated with RNA, isolated from a biological sample, under conditions of temperature and ionic strength that promote base pairing between the probe and target Zsig98 RNA species. After separating unbound probe from hybridized molecules, the amount of hybrids is detected.

Well-established hybridization methods of RNA detection include northern analysis and dot/slot blot hybridization (see, for example, Ausubel (1995) at pages 4-1 to 4-27, and Wu et al. (eds.), “Analysis of Gene Expression at the RNA Level,” in Methods in Gene Biotechnology, pages 225-239 (CRC Press, Inc. 1997)). Nucleic acid probes can be detectably labeled with radioisotopes such as ³²P or ³⁵S. Alternatively, Zsig98 RNA can be detected with a nonradioactive hybridization method (see, for example, Isaac (ed.), Protocols for Nucleic Acid Analysis by Nonradioactive Probes (Humana Press, Inc. 1993)). Typically, nonradioactive detection is achieved by enzymatic conversion of chromogenic or chemiluminescent substrates. Illustrative nonradioactive moieties include biotin, fluorescein, and digoxigenin.

Zsig98 oligonucleotide probes are also useful for in vivo diagnosis. As an illustration, ¹⁸F-labeled oligonucleotides can be administered to a subject and visualized by positron emission tomography (Tavitian et al., Nature Medicine 4:467 (1998)).

Numerous diagnostic procedures take advantage of the polymerase chain reaction (PCR) to increase sensitivity of detection methods. Standard techniques for performing PCR are well-known (see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

One variation of PCR for diagnostic assays is reverse transcriptase-PCR (RT-PCR). In the RT-PCR technique, RNA is isolated from a biological sample, reverse transcribed to cDNA, and the cDNA is incubated with Zsig98 primers (see, for example, Wu et al. (eds.), “Rapid Isolation of Specific cDNAs or Genes by PCR,” in Methods in Gene Biotechnology, pages 15-28 (CRC Press, Inc. 1997)). PCR is then performed and the products are analyzed using standard techniques.

As an illustration, RNA is isolated from biological sample using, for example, the guanidinium-thiocyanate cell lysis procedure described above. Alternatively, a solid-phase technique can be used to isolate mRNA from a cell lysate. A reverse transcription reaction can be primed with the isolated RNA using random oligonucleotides, short homopolymers of dT, or Zsig98 anti-sense oligomers. Oligo-dT primers offer the advantage that various mRNA nucleotide sequences are amplified that can provide control target sequences. Zsig98 sequences are amplified by the polymerase chain reaction using two flanking oligonucleotide primers that are typically 20 bases in length.

PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled Zsig98 probe, and examined by autoradiography. Additional alternative approaches include the use of digoxigenin-labeled deoxyribonucleic acid triphosphates to provide chemiluminescence detection, and the C-TRAK colorimetric assay.

Another approach for detection of Zsig98 expression is cycling probe technology (CPT), in which a single-stranded DNA target binds with an excess of DNA-RNA-DNA chimeric probe to form a complex, the RNA portion is cleaved with RNAase H, and the presence of cleaved chimeric probe is detected (see, for example, Beggs et al., J. Clin. Microbiol. 34:2985 (1996), Bekkaoui et al., Biotechniques 20:240 (1996)). Alternative methods for detection of Zsig98 sequences can utilize approaches such as nucleic acid sequence-based amplification (NASBA), cooperative amplification of templates by cross-hybridization (CATCH), and the ligase chain reaction (LCR) (see, for example, Marshall et al., U.S. Pat. No. 5,686,272 (1997), Dyer et al., J. Virol. Methods 60:161 (1996), Ehricht et al., Eur. J. Biochem. 243:358 (1997), and Chadwick et al., J. Virol. Methods 70:59 (1998)). Other standard methods are known to those of skill in the art.

Zsig98 probes and primers can also be used to detect and to localize Zsig98 gene expression in tissue samples. Methods for such in situ hybridization are well-known to those of skill in the art (see, for example, Choo (ed.), In Situ Hybridization Protocols (Humana Press, Inc. 1994), Wu et al. (eds.), “Analysis of Cellular DNA or Abundance of mRNA by Radioactive In Situ Hybridization (RISH),” in Methods in Gene Biotechnology, pages 259-278 (CRC Press, Inc. 1997), and Wu et al. (eds.), “Localization of DNA or Abundance of mRNA by Fluorescence In Situ Hybridization (RISH),” in Methods in Gene Biotechnology, pages 279-289 (CRC Press, Inc. 1997)). Various additional diagnostic approaches are well-known to those of skill in the art (see, for example, Mathew (ed.), Protocols in Human Molecular Genetics (Humana Press, Inc. 1991), Coleman and Tsongalis, Molecular Diagnostics (Humana Press, Inc. 1996), and Elles, Molecular Diagnosis of Genetic Diseases (Humana Press, Inc., 1996)).

### 15. Detection of Zsig98 Protein with Anti-Zsig98 Antibodies

The present invention contemplates the use of anti-Zsig98 antibodies to screen biological samples in vitro for the presence of Zsig98, and particularly for the upregulation of Zsig98. In one type of in vitro assay, anti-Zsig98 antibodies are used in liquid phase. For example, the presence of Zsig98 in a biological sample can be tested by mixing the biological sample with a trace amount of labeled Zsig98 and an anti-Zsig98 antibody under conditions that promote binding between Zsig98 and its antibody. Complexes of Zsig98 and anti-Zsig98 in the sample can be separated from the reaction mixture by contacting the complex with an immobilized protein which binds with the antibody, such as an Fc antibody or Staphylococcus protein A. The concentration of Zsig98 in the biological sample will be inversely proportional to the amount of labeled Zsig98 bound to the antibody and directly related to the amount of free-labeled Zsig98.

Alternatively, in vitro assays can be performed in which anti-Zsig98 antibody is bound to a solid-phase carrier. For example, antibody can be attached to a polymer, such as aminodextran, in order to link the antibody to an insoluble support such as a polymer-coated bead, a plate or a tube. Other suitable in vitro assays will be readily apparent to those of skill in the art.

In another approach, anti-Zsig98 antibodies can be used to detect Zsig98 in tissue sections prepared from a biopsy specimen. Such immunochemical detection can be used to determine the relative abundance of Zsig98 and to determine the distribution of Zsig98 in the examined tissue. General immunochemistry techniques are well established (see, for example, Ponder, “Cell Marking Techniques and Their Application,” in Mammalian Development: A Practical Approach, Monk (ed.), pages 115-38 (IRL Press 1987), Coligan at pages 5.8.1-5.8.8, Ausubel (1995) at pages 14.6.1 to 14.6.13 (Wiley Interscience 1990), and Manson (ed.), Methods In Molecular Biology, Vol. 10: Immunochemical Protocols (The Humana Press, Inc. 1992)).

Immunochemical detection can be performed by contacting a biological sample with an anti-Zsig98 antibody, and then contacting the biological sample with a detectably labeled molecule that binds to the antibody. For example, the detectably labeled molecule can comprise an antibody moiety that binds to anti-Zsig98 antibody. Alternatively, the anti-Zsig98 antibody can be conjugated with avidin/streptavidin (or biotin) and the detectably labeled molecule can comprise biotin (or avidin/streptavidin). Numerous variations of this basic technique are well-known to those of skill in the art.

Alternatively, an anti-Zsig98 antibody can be conjugated with a detectable label to form an anti-Zsig98 immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below.

The detectable label can be a radioisotope that is detected by autoradiography. Isotopes that are particularly useful for the purpose of the present invention are ³H, ¹²⁵I, ¹³¹I, ³⁵S and ¹⁴C.

Anti-Zsig98 immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-Zsig98 immunoconjugates can be detectably labeled by coupling an antibody component to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-Zsig98 immunoconjugates of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-Zsig98 immunoconjugates can be detectably labeled by linking an anti-Zsig98 antibody component to an enzyme. When the anti-Zsig98-enzyme conjugate is incubated in the presence of the appropriate substrate, the enzyme moiety reacts with the substrate to produce a chemical moiety, which can be detected, for example; by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels, which can be employed in accordance with the present invention. The binding of marker moieties to anti-Zsig98 antibodies can be accomplished using standard techniques known to the art. Typical methodology in this regard is described by Kennedy et al., Clin. Chim. Acta 70:1 (1976), Schurs et al., Clin. Chim. Acta 81:1 (1977), Shih et al., Int'l J. Cancer 46:1101 (1990), Stein et al., Cancer Res. 50:1330 (1990), and Coligan, supra.

Moreover, the convenience and versatility of immunochemical detection can be enhanced by using anti-Zsig98 antibodies that have been conjugated with avidin, streptavidin, and biotin (see, for example, Wilchek et al. (eds.), “Avidin-Biotin Technology,” Methods In Enzymology, Vol. 184 (Academic Press 1990), and Bayer et al., “Immunochemical Applications of Avidin-Biotin Technology,” in Methods In Molecular Biology, Vol. 10, Manson (ed.), pages 149-162 (The Humana Press, Inc. 1992).

Methods for performing immunoassays are well-established. See, for example, Cook and Self, “Monoclonal Antibodies in Diagnostic Immunoassays,” in Monoclonal Antibodies: Production, Engineering, and Clinical Application, Ritter and Ladyman (eds.), pages 180-208, (Cambridge University Press, 1995), Perry, “The Role of Monoclonal Antibodies in the Advancement of Immunoassay Technology,” in Monoclonal Antibodies: Principles and Applications, Birch and Lennox (eds.), pages 107-120 (Wiley-Liss, Inc. 1995), and Diamandis, Immunoassay (Academic Press, Inc. 1996).

In a related approach, biotin- or FITC-labeled Zsig98 can be used to identify cells that bind Zsig98. Such can binding can be detected, for example, using flow cytometry.

The present invention also contemplates kits for performing an immunological diagnostic assay for Zsig98 gene expression. Such kits comprise at least one container comprising an anti-Zsig98 antibody, or antibody fragment. A kit may also comprise a second container comprising one or more reagents capable of indicating the presence of Zsig98 antibody or antibody fragments. Examples of such indicator reagents include detectable labels such as a radioactive label, a fluorescent label, a chemiluminescent label, an enzyme label, a bioluminescent label, colloidal gold, and the like. A kit may also comprise a means for conveying to the user that Zsig98 antibodies or antibody fragments are used to detect Zsig98 protein. For example, written instructions may state that the enclosed antibody or antibody fragment can be used to detect Zsig98. The written material can be applied directly to a container, or the written material can be provided in the form of a packaging insert.

The present invention, thus generally described, will be understood more readily by reference to the following examples, which are provided by way of illustration and are not intended to be limiting of the present invention.

### 16. Examples

### Example 1

### Nothern Blot Expression of zsig98

Sense primer zc39486 (SEQ ID NO: 9) and antisense primer zc39487 (, SEQ ID NO: 10) were used in a 50 ul PCR reaction to generate a 120 bp fragment for use in northern blots as follows: 10 ul 10× Advantage 2 buffer and 2 ul Advantage 2 polymerase mix (BD Biosciences, Clontech, Palo Alto, Calif.), 10 ul Redi-Load (Invitrogen, Carlsbad, Calif.), 8 ul 2.5 mM dNTPs (Applied Biosystems, Foster City, Calif.) 2 ul 20 pm/ul each zc39486 and zc39487, 0.5 ul 0.293 ug/ul zsig98 full length cDNA, image clone ID #4650644, and H2O to 100 ul. Cycling conditions were 1 cycle at 94° C. 2′, 35 cycles at 94° C. 20″, 54° C. 20″, 72° C. 30″, followed by one cycle at 72° C. 7′, and a hold at 4° C. The reaction was run in an agarose gel and the fragment was purified using a Qiagen gel purification column (Qiagen, Valencia, Calif.) according to the manufacturer's instructions. The fragment was quantitated by a spectrophotometer reading. 50 ng of fragment was labeled using Prime-It II reagents (Stratagene, LaJolla, Calif.) according to the manufacturer's instructions, and separated from unincorporated nucleotides using an S-200 microspin column (Amersham, Piscataway, N.J.) according to the manufacturer's protocol. Blots to be probed with zsig98 (Multiple Tissue Northern Blots I, II, and III, Multiple Tissue Expression Array, and the Multiple Fetal Tissue Northern Blot, all from BD Biosciences, Clontech, Palo Alto, Calif.) were prehybridized overnight at 55° C. in ExpressHyb (BD Biosciences, Clontech Palo Alto, Calif.) in the presence of 100 ug/ml salmon sperm DNA (Stratagene, La Jolla, Calif.) and 6 ug/ml cot I DNA (Invitrogen, Carlsbad, Calif.) which were boiled and snap-chilled prior to adding to the blots. Radiolabelled zsig98, salmon sperm DNA and cot1 DNA were mixed together and boiled 5′, followed by a snap chilling on ice. Final concentrations of the salmon sperm DNA and cot1 DNA were as in the prehybridization step and the final concentration of radiolabelled zsig98 was 1×106 cpm/ml. Blots were hybridized overnight in a roller oven at 55° C., then washed copiously at RT in 2×SSC, 0.1% SDS, with several buffer changes, then at 65° C. The final wash was at 65° C. in 0.1×SSC, 0.1% SDS. Blots were then exposed to film with intensifying screens for 1 day followed by 7 days. The Multiple Tissue Northern and Fetal Multiple Tissue Northern Blots were then probed with a transferrin receptor probe, generated as follows: sense primer zc10565 (SEQ ID NO: 11) and antisense primer zc10651 (SEQ ID NO: 12) were used in a 50 ul PCR reaction with 5 ul 10× Advantage 2 buffer, 1 ul Advantage 2 cDNA polymerase mix (BD Biosciences, Clontech, Palo Alto, Calif.), 5 ul 10× Redi-Load (Invitrogen, Carlsbad Calif.), 4 μl 2.5 mM dNTPs (Applied Biosystems, Foster City, Calif.), 1 ul each zc10565 and zc10651, and 5 ul placenta marathon™ cDNA (BD Biosciences, Clontech, Palo Alto, Calif.). Cycling conditions were one cycle at 94° C., 2′, 35 cycles of 94° C. 20″ 57° C. 20″ 72° C. 45″, one cycle at 72° C. 7′, followed by a 4° C. hold. The reaction was run in an agarose gel and the fragment was purified using Qiagen gel purification columns (Qiagen, Valencia, Calif.) according to the manufacturer's instructions. The fragment was quantitated by a spectrophotometer reading. The transferrin receptor fragment was labeled and used to probe the Multiple Tissue Northern Blots and the Fetal Tissue Northern blot as described above. Blots were exposed to film with intensifying screens for 8 days. The transferrin receptor control probing experiment shows the blots were of good quality and a low to moderately expressed control gene could be observed with an 8-day exposure.

Zsig98 mRNA, is clearly observed in several tissues from the Multiple Tissue Northern blots, the highest expression being in pancreas, followed by brain and testis. The transcript size is ˜800-900 bp. All other tissues appear to contain at least some zsig98 mRNA, with higher levels seen in spinal cord, heart, placenta, lung, liver, kidney, and adrenal gland. The Fetal Multiple Tissue Northern Blot shows zsig98 expression is highest in brain, but visible in fetal liver, lung and kidney. The results from the Multiple Tissue Expression Array Dot Blot show that zisg98 mRNA is observed in several brain tissues, the highest being the substantia nigra, left cerebellum, corpus callosum, right cerebellum, whole brain, cerebral cortex, accumbens nucleus, and thalamus. Other tissues where zsig98 can be seen on this dot blot are fetal brain, spinal cord, pituitary, stomach, and testis.

### Example 2

### Construction of Mammalian Expression Vectors that Expresses Human Zsig98 Polypeptide

An expression vector was prepared for the expression of human zsig98 polypeptide, zsig98CHIS, wherein the construct was designed to express a zsig98 polypeptide comprised of the initiating methionine to the last amino acid minus the stop codon (SEQ ID NO: 22) and with a C-terminal HIS tag, (SEQ ID NO:13).

A 285 bp PCR-generated zsig98 DNA fragment was created using ZC48711, (SEQ ID NO:14) and ZC48196 (SEQ ID NO:15) as PCR primers (to add Esp3I restriction sites) and Advantage II reagents (Becton Dickinson, Franklin Lakes, N.J.) with 10% DMSO (Sigma, ST. Louis, Mo.). A plasmid containing the zsig98 cDNA was used as a template. PCR amplification of the zsig98 fragment was performed as follows: PCR amplification of the zsig98 fragment was performed as follows: One cycle of 94 C for 2 minutes; then thirty cycles at 94° C. for 30 seconds, 55° C. for 30 seconds, 72° C. for 1.5 minutes, followed by one cycle of 72° C. for 7 minutes and then a 4° C. hold. The reaction was purified using QIAquick PCR purification kit (Qiagen, Santa Clarita, Calif.) and digested with Esp3I (Fermentas, Hanover, Md.) following manufacturer's protocol. The reaction was purified using QIAquick PCR purification kit (Qiagen, Santa Clarita, Calif.) according the manufacturer's instructions.

The excised DNA was subcloned into an expression vector which had been cut with Eco31I (Fermentas, Hanover, Md.). The expression vector used the native zsig98 signal peptide and attached the HIS tag to the Zsig98 polypeptide-encoding polynucleotide sequence. About 10 μl of the restriction digested zsig98 insert and about 75 ng of the digested vector were ligated using the Fast link ligation kit (EPICENTRE technologies (Madison, Wis.). Two microliter of the ligation reaction was transformed into ElectroMax DH10B competent cells (Invitrogen, Carlsbad, Calif.) according to manufacturer's direction and plated onto LB plates containing 25 μg/ml Kanamycin, and incubated overnight. Colonies were submitted for sequencing in 5 ml liquid cultures of individual colonies. The insert sequence of clones was verified by sequence analysis.

A LR reaction was set up using LR reaction kit (Invitrogen, Carlsbad, Calif.), about 300 ng of another expression vector and about 100-300 ng of zsig98/expression vector entry clone. The reaction contained 4 μl 5×LR reaction buffer, 1 μl of Topoisomerase, 4 μl of LR Clonase enzyme mix and TE buffer for a final volume of 20. Incubated for 1 hour at 25° C., then 2 μl proteinase K added and incubated at 37° C. for 10 minutes. One microliter of the LR reaction was transformed into ElectroMax DH10B competent cells (Invitrogen, Carlsbad, Calif.) according to manufacturer's direction and plated onto LB plates containing 50 μg/ml Kanamycin, and incubated overnight. Colonies were screened by PCR and simultaneously inoculating 100 μl of LB broth.

The PCR was set up using the following: Advantage 2 reagents (BD Biosciences Clontech, Palo Alto, Calif.) and ZC5020 (SEQ ID NO: 16) and ZC14063 (SEQ ID NO: 17) as PCR primers. PCR amplification of the zsi98 was performed as follows: One cycle of 94 C for 2 minutes; then 35 cycles at 94° C. for 30 seconds, 62° C. for 30 seconds, 72° C. for 2 minute, followed by one cycle of 72° C. for 5 minutes and then a 4° C. hold. A band of the predicted size 564 bp was visualized by agarose gel electrophoresis. 5 ml liquid culture was inoculated with the 100 μl LB clone mix and left overnight at 37° C. with shaking. Glycerol stock archieved at −80° C. Plate was struck with glycerol stock and left overnight at 37° C. A 5 ml liquid culture was inoculated with clone and left overnight at 37° C. with shaking. 5 ml overnight culture used to inoculate 500 ml of liquid culture, left overnight at 37° C. with shaking.

A mega prep was done using a QIAfilter plasmid mega kit (Qiagen, Santa Clarita, Calif.) according to an optimized protocols based on manufacturer's instructions.

### Example 3

### Expression and Purification of the Zsig98 Construct

A. Expression of Zsig98 in 293T Cells

Zsig98 can be expressed transiently in 293T cells (Stanford University School of Medicine, Stanford, Calif., ATCC (SD-3515)) to generate initial purified protein. The day before the transfection, 293T cells are seeded at 6.5×10⁴ cells/cm² in 30 T162 culture flasks with a total volume of 30 ml of culture media (SL7V4+5% FBS+1% Pen/Strep) per flask. The cells are allowed to incubate for 24 hours at 37° C.

A DNA/Liposome mixture is prepared as follows: Two 50 ml conical tubes are filled with 25 mLs of transfection media (SL7V4+1% Pen/Strep) and an amount of an expression vector containing the Zsig98 gene is added to each. A separate set of two 50 ml conical tubes are filled with 22 ml of transfection media (above) and 3 ml of liposomes (Lipofectamine, Gibco) is added to each. For each set of tubes, one tube of DNA is added to one tube of liposomes and the DNA/liposome mix is incubated for 30 minutes. The two 50 ml conical tubes containing the DNA/liposome mixtures are pooled (about 100 ml) and 300 ml of transfection media is added.

The 30 flasks of the 293T cells are decanted, washed 1× with about 15 ml of PBS; and 12.5 ml of the diluted DNA/liposome mixture is added to each flask. The flasks are incubated for 3 hours at 37° C. After the incubation period, 25 ml of culture media (above) are added to each T162 flask. The transfection media is harvested after approximately 96 hours and was used for protein purification.

B. Expression of Zsig98 in BHK Cells

Zsig98 protein can be produced in BHK cells transfected with a vector containing the Zsig98 gene. BHK 570 cells (ATCC CRL-10314) are plated in T75 tissue culture flasks and allowed to grow to approximately 50 to 70% confluence at 37° C., 5% CO₂, in growth media (SL7V4, 5% FBS, 1% pen/strep). The cells are then transfected with zsig98-CEE/pZMP21 by liposome-mediated transfection (using Lipofectamine™; Life Technologies), in serum free (SF) media (SL7V4). The plasmid (16 μg) is diluted into 1.5 ml tubes to a total final volume of 640 μl with SF media. Thirty-five microliters the lipid mixture is mixed with 605 μl of SF medium, and the resulting mixture is allowed to incubate approximately 15 minutes at room temperature. Five milliliters of SF media is then added to the DNA:lipid mixture. The cells are rinsed once with 10 ml of PBS, the PBS is decanted, and the DNA:lipid mixture is added. The cells are incubated at 37° C. for five hours, then 15 ml of media (SL7V4, 5% FBS, 1% pen/strep) is added to each plate. The plates are incubated at 37° C. overnight, and the DNA:lipid media mixture is replaced with selection media (SL7V4, 5% FBS, 1% pen/strep, 1M methotrexate) the next day. Approximately 10 days post-transfection, methotrexate-resistant colonies from the T75 transfection flask are trypsinized, and the cells are pooled and plated into a T-162 flask and transferred to large-scale culture.

C. Purification of Zsig98-CEE from 293T Cells

Unless otherwise noted, all operations are carried out at 4° C. The following procedure is used for purifying Zsig98 containing C-terminal Glu-Glu (EE) tags. Conditioned media from 293T cells expressing Zsig98-CEE is purified. Total target protein concentrations of the conditioned media are determined via SDS-PAGE and Western blot analysis with the anti-EE antibody.

A 5.5 ml column of anti-EE Poros 50 A (PE BioSystems, Framingham, Mass.) (prepared as described below) is poured in a Waters AP-1, 1 cm×7 cm glass column (Waters, Milford, Mass.). The column is flow packed and equilibrated on a BioCad Sprint (PE BioSystems, Framingham, Mass.) with phosphate buffered saline (PBS) pH 7.4. The conditioned media is adjusted with NaCl to 0.3 M and the pH adjusted to 7.2. The conditioned media is then loaded on the column overnight with about 3 ml/minute flow rate. The column is washed with 10 column volumes (CVs) of PBS pH 7.4, and again washed with 3CVs 5× Sigma PBS pH 7.4. It is step eluted with 0.5 M Acetate, 0.5 M NaCl, pH 2.5 at 3 ml/minute. The fraction tubes contain 1 ml Tris base (no pH adjustment) to neutralize the elution immediately. The column is again washed for 2CVs with 5× Sigma PBS, pH 7.4 to neutralize the column and then equilibrated in PBS (pH 7.4). Two ml fractions are collected over the entire elution chromatography and absorbance at 280 and 215 nM are monitored; the pass through and wash pools are also saved and analyzed. The 5×PBS and the acid elution peak fractions are analyzed for the target protein via SDS-PAGE Silver staining and Western Blotting with the primary antibody anti-EE and secondary antibody, anti mouse-HRP conjugated. The acid elution fractions of interest are pooled and concentrated from 38 ml to 0.8 ml using a 5000 Dalton molecular weight cutoff membrane spin concentrator (Millipore, Bedford, Mass.) according to the manufacturer's instructions.

To separate Zsig98-CEE from aggregated material and any other contaminating co-purifying proteins, the pooled concentrated fractions are subjected to size exclusion chromatography on a 1.6×60 cm (120 ml) Superdex 75 (Pharmacia, Piscataway, N.J.) column equilibrated and loaded in PBS at a flow rate of 1.0 ml/min using a BioCad Sprint. Three ml fractions are collected across the entire chromatography and the absorbance at 280 and 215 nM are monitored. The peak fractions are characterized via SDS-PAGE Silver staining, and only the most pure fractions are pooled.

On Western blotted, Coomassie Blue and Silver stained SDS-PAGE gels, the Zsig98-CEE band is identified. The protein concentration of the purified material is performed by BCA analysis (Pierce, Rockford, Ill.) and the protein is aliquoted, and stored at −80° C. according to standard procedures.

To prepare PorosA50 anti-EE, a 65 ml bed volume of Poros A50 (PE Biosystems) is washed with 100 ml of water and then 0.1 M triethanolamine, pH 8.2 (TEA, ICN, Aurora, Ohio), 1 M Na₂SO₄, pH 8.8 containing 0.02% sodium azide using a vacuum flask filter unit. The EE monoclonal antibody solution, at a concentration of 2 mg/ml in a volume of 300 ml, is mixed with the washed resin in a volume of 250 ml. After an overnight incubation at room temperature, the unbound antibody is removed by washing the resin with 5 volumes of 200 mM TEA, 1 M Na₂S0₄, pH 8.8 containing 0.02% sodium azide as described above. The resin is resuspended in 2 volumes of TEA, 1 M Na₂S0₄, pH 8.8 containing 0.02% sodium azide and transferred to a suitable container. Three ml of 25 mg/ml (68 mM) Disuccinimidyl suberate (in DMSO supplied by Pierce, Rockford, Ill.) is added and the solution is incubated for three hours at room temperature. Nonspecific sites on the resin are then blocked by incubating for 10 min at room temperature with 5 volumes of 20 mM ethanolamine (Sigma, St. Louis, Mo.) in 200 mM TEA, pH 8.8 using the vacuum flask filter unit. The resin is washed with PBS, pH 7.4, followed by 0.1 M Glycine, pH 3 and then neutralized with 10×PBS. After washing with distilled water, the final coupled anti-EE Poros-A 50 resin is stored at 4° C. in 20% Ethanol.

### Example 4

### Determination of N-Terminal Sequence and Mature Protein

A protein sample was supplied in 50 mM NaPO4, 109 mM NaCl, 0.1% BSA pH 7.3 at 0.01 mg/ml (Quantitative Western). The protein was lyophilized a whole 100 ul vial to dryness and resuspended in 38 ul 1× reducing NuPAGE sample buffer and heated on boiling water bath for 5 minutes. The sample was run on 4-12% bis tris MES NuPAGE, transferred to PVDF, and coomassie blue stained. The upper band associated with the triplet bands at approximately 10 kDa was excised for sequencing. The first 20 amino acids were sequenced and the N-terminal of the expressed mature protein was identified as Glutamine 35. Thus, the mature Zsig98 protein comprises or consists of the protein as shown in SEQ ID NO: 18. N-term sequencing indicated a modification on S5 of SEQ ID NO: 18.

### Example 5

### Determination of Modifications of Mature Zsig98 Protein

Modifications of the Zsig98 protein from the 293 cell expression product were determined as follow: expressed protein was run on a gel, and bands with a molecular weight less than 14 kDa were separated. The protein was reduced, alkylated and digested with trypsin.

One hundred uL of expressed protein was dried down in a speed vac to ˜20 uL. The entire volume was loaded onto lane 1 on a gel and separated. Five bands were targeted for digestion, all with a molecular weight less than 14 kDa. Band 1 contained the heaviest protein, band 5 the lightest, and band 2 contained the most protein.

TABLE 5

Expected tryptic peptides of zsig98

| | | | | | | | |
|---|---|---|---|---|---|---|---|
| Num | From | To | Mass | MH+ | M2H+ | M3H+ | Sequence |

| | | | | | | | |
|---|---|---|---|---|---|---|---|
| T1 | 1 | 25 | 2844.25 | 2845.26 | 1423.13 | 949.09 | EPAGSAVPAQSRPCVDCHAFEFMQR |
| T2 | 26 | 31 | 714.4 | 715.41 | 358.21 | 239.14 | ALQDLR |
| T3 | 32 | 32 | 146.11 | 147.11 | 74.06 | 49.71 | K |
| T4 | 33 | 40 | 891.4 | 892.41 | 446.71 | 298.14 | TACSLDAR |
| T5 | 41 | 50 | 1172.64 | 1173.65 | 587.33 | 391.89 | TETLLLQAER |
| T6 | 51 | 51 | 174.11 | 175.12 | 88.06 | 59.05 | R |
| T7 | 52 | 74 | 2420.99 | 2422 | 1211.5 | 808 | ALCACWPAGHGSGSGGGHHHHHH |

LC-MS analysis identified that the modified residue is contained in band 2. LC-MS/MS analysis of the peptide containing the modified residue demonstrated that zsig98 is O-glycosylated at S5 of SEQ ID NO: 18. The glycosylation contains two sialic acid, one hexose and one HexNAc unit. Multiple bands were identified indicating that the Zsig98 forms dimmers and/or multimers.

### Example 6

### Native Ligation of Mature Zsig98 Polypeptide

Native Ligation is a well published technique, and involves the ligation of peptide fragments through a C-terminal thioester and a N-terminal cysteine under near neutral pH with fully deprotected peptides and the option of guanidine hydrochloride in the ligation buffer. See Novabiochem. 2002. Reagents for Peptide Ligation and Labeling; Hilvert D. Quaderer R. Org. Let. 3 (20): 3181-3184, 2001; Shin et al. J. Am. Chem. Soc. 121: 11684-11689, 1999; Pessi et al. J. Am. Chem. Soc.: 121 11369-11374, 1999; Bulaj G. Biotech. Advances.: 20 87-92, 2005; and Fox et al. Protein Science. 14:1818-1826, 2005.

C terminal zsig98 peptides [PEP05008 and PEP05011, both having the amino acid sequence of SEQ ID NO: 15, C69-H95) were synthesized on an ABI 433A Peptide Synthesizer using the FastMoc 0.25 mmole synthesis, with standard Fmoc chemistry. It was synthesized on Fmoc-His(trt)-Wang resin [Anaspec Inc.] Cleavage of the peptide from the resin, as well as the side chain protecting groups was performed by the addition of 0.5 mL H2O, 0.5 mL Ethyl Methyl Sulfide, 0.25 mL Ethane dithiol, 0.5 mL triisopropyl silane, 0.75 mL phenol (heated to 60° C.) and 12.0 mL Trifluoroacetic acid. These chemistries were allowed to react under Ar gas for 4 hours, resin was then filtered and washed with TFA and DCM. TFA and DCM were then dried off in vacuo. Room temperature Diethyl ether was then added to precipitate the peptide. Peptide was filtered (Whatman 2V 32 cm), and resuspended in 20% ACN 1% Acetic acid and filtered through a 20 μm ZapCap. Crude product was then subjected to analytical RP HPLC and MS. It was then purified on a Waters DeltaPrep equipped with a 50×250 mm Vydac C18 column (A=0.050% TFA in H2O, B=0.040% TFA in ACN 10-80% B over 40 min, room temp) and collected in fractions. Selected fractions were screened through analytical RP HPLC on 4.6×250 mm Vydac C18 column at 40° C. (A=0.050% TFA in H2O, B=0.040% TFA in ACN, 2-82% B over 20 min). Fractions that met screening criteria (peptide >95% pure) were then pooled, and lyophilized to dryness, resuspended in 20% ACN 1% Acetic acid into a pre weighed 50 mL falcon tube and again lyophilized to dryness. Final mass of peptide was determined by differences of the 2 masses of the falcon tube.

N terminal zsig98 peptides [PEP05009 and PEP05013, both having the amino acid sequence of SEQ ID NO: 16, E35-A68] were synthesized on an ABI 433A Peptide synthesizer using the FastMoc 0.25 mmole synthesis, with standard Fmoc chemistry. The peptides were synthesized on a H-Ala-Sulfamylbutyryl NovaSyn TG resin [Novabiochem]. Activation of the specialized ‘safety catch’ resin lasted for 2 hours at room temperature under Ar (gas) and was performed with 1 M trimethylsilyl-diazomethane in THF. Several washes were performed with both THF and DMF by syringe. The activated resin was then cleaved from the peptide by and adding a catalytic amount of sodium benzenethiol salt (1× mole over peptide), and ethyl-3-mercaptopropionate (50× moles over peptide) in DMF (14 mL). After a 24 hour room temperature cleavage under Ar (gas) the resin was filtered away from the peptide (Whatman Glass fiber filter GF/D), the DMF was rid of by lyophilization, and the gel like peptide was treated with the standard deprotection protocol as in the section above. The peptide was rid of scavengers and TFA in vacuo and was precipitated with diethyl ether. It was then resuspended in 10% ACN 1% acetic acid and was purified on a Waters DeltaPrep equipped with a 50×250 mm Vydac C18 column (A=0.050% TFA in H2O, B=0.040% TFA in ACN 10-80% B over 40 min, room temp) and collected in fractions. Selected fractions were screened through analytical RP HPLC on 4.6×250 mm Vydac C18 column at 40° C. Fractions that met screening criteria (as above) were then pooled, and lyophilized to dryness, resuspended in 10% ACN 1% Acetic Acid into a pre weighed 50 mL falcon tube and lyophilized to dryness. Final mass of peptide was determined by the differences of the two masses of the falcon tube.

Mature Zsig98 [PEP05014 and PEP05015, both having the amino acid sequence of SEQ ID NO: 17) was created by the ligation of the C terminal peptide, and the N terminal Peptide thioester. The peptides were suspended in a solution composed of 6 M Gdn HCl, 100 mM TRIS, 50 mM TCEP and 1% (v/v) benzenethiol. Peptides themselves had buffering characteristic, thus the Solution was not put to a preset pH, the addition of the peptides changed the pH to a desired level (˜8.5) which was above the pI of both peptides. Each peptide was suspended independently, so that the overall molarity of the limiting peptide when mixed would be ˜1 mM, and non limiting anywhere from 1.2-2× over the limiting reagent concentration. The reaction was stopped at time points by adding a half volume of the reaction aliquot of 4% BME and allowing to reduce for 5 minutes, then the addition of a half volume of 40% HAc. The reaction was monitored by C18 RP-HPLC (A=0.050% TFA in H2O, B=0.040% TFA in ACN, 30 min isocratic at 20% B then 20-55% B over 35 min at 40° C.), and when limiting reagent peak was diminished to insignificance, the reaction was stopped by using the afore mentioned BME/HAc method. The entirety of the stopped reaction was then loaded onto a Waters DeltaPrep equipped with a 50×250 mm Vydac C4 RP column (A=0.050% TFA in H2O, B=0.040% TFA in ACN, 20 min isocratic at 30% B at 120 mL/min then 30-95% B over 32.5 at 50 mL/min, room temp). Collected fractions were screened with analytical C18 RP HPLC, and those fractions that passed the screening criteria (as above) were pooled, analyzed by MS and HPLC and lyophilized to dryness. The dry product was suspended in 10% ACN 1% HAc into a pre weighed 50 mL falcon tube and lyophilized to dryness. Product was weighed and entered into the peptide synthesis database.

### Example 7

### Refolding of Synthetically Produced Mature Zsig98 Polypeptide

The protein produced synthetically in Example 6 above was refolded as follows.

The Refold Buffer contained 55 mM Tris, pH 8.2 (cold); 0.55M Arginine; 10.56 mM NaCl; 0.44 mM KCl; 2 mM CaCl2; 2 mM MgCl2; 0.055% PEG 3350; 1.0 mM Reduced Glutathione; and 0.1 mM Oxidized Gllutathione. Two hundred milliliters of the refold buffer was prepared and 13 mg of the zsig98 peptide was added to glutathione redox buffer to a final concentration of 0.065 mg/ml. Incubate the solution with stirring at 4° C. O/N. The peptide should be added slowly in increments. Progress of the reaction was ronitored by analytical RP HPLC. The reaction was quenched by acidification, and the peptide was concentrated in 1K centrifugal devices to formulate the product by SEC (Superdex Peptide.) Concentration of the protein was very slow.

An aliquot of the resulting refolded protein was run on a SDS PAGE gel and multimers, including dimers, were observed.

In a separate refolding procedure, 200 ml refold buffer containing 0.2M Tris; 1 mM EDTA; 1 mM GSH; and 1 mM GSSG pH 8.2 was prepared and 10 mg of the zsig98 peptide was added slowly to glutathione redox buffer to a final concentration of 0.05 mg/ml. The solution was incubated with stirring at 4° C. O/N and the progress of the reaction was monitored by analytical HPLC. The pepide was lyophilized, then redissolved in pH 5.2 30 mM Sodium Acetate and purified the product by SEC (Superdex Peptide).

### Example 8

Transfection of 293 Cells with Zsig 98

C-terminal His-tagged zsig98 was produced transiently in 293F cells (Invitrogen, Carlsbad, Calif. Cat# R790-07). Briefly, 293F suspension cells were cultured in 293 Freestyle medium (Invitrogen, Carlsbad, Calif. Cat# 12338-018) at 37 C, 6% CO2 in a 3 L spinner at 95 RPM. Fresh medium was added immediately prior to transfection to obtain a 1.5 liter working volume at a final density of 1×10E6 cells/ml. 2.0 ml of Lipofectamine 2000 (Invitrogen, Carlsbad, Calif. Cat# 11668-019) was added to 20 ml Opti-MEM medium (Invitrogen, Carlsbad, Calif. Cat# 31985-070) and 1.5 mg of construct DNA was diluted in a separate tube of 20 ml Opti-MEM. Each tube was incubated separately at room temperature for 5 minutes, then combined and incubated together for an additional 30 minutes at room temperature with occasional gentle mixing. The lipid-DNA mixture was then added to the spinner of 293F cells which were returned to 37 C, 6% CO2 at 75 RPM. After approximately 96 hours, the conditioned medium was harvested and 0.2 μM filtered.

### Example 9

### Enrichment of C-Terminally 6×His Tagged Protein from 293 Transient System

Fourteen hundred milliliters of 0.22 um filtered 293 media expressing Zsig98 can be concentrated to <50 mL using a peristaltic pump system outfitted with 1×0.1 m2 5 kD MWCO regenerated cellulose membrane (Millipore). Concentrated media is adjusted to 0.5M NaCl via addition of 0.4M solid (JT Baker) and 25 mM Imidazole via dilution of 2.5M stock solution. The pH is adjusted to 7.5 using HCl. The adjusted media is combined with 1 mL of Ni NTA His Bind Superflow resin (Novagen) and allowed to batch bind overnight at 4° C. while gently rocking.

The following morning the slurry is transferred to an gravity flow econo-column (BioRAD), and the flow through is collected. The resin is washed with at least 30 CV of 50 mM NaPO4, 500 mM NaCl, 25 mM Imidazole, pH 7.5, and the wash fraction is collected by gravity flow. Bound protein is eluted via competition using the Histidine analog Imidazole. Gravity flow is stopped, and 15CV of 50 mM NaPO4, 500 mM NaCl, 500 mM Imidazole pH 7.5 is added, making sure the resin is fully suspended. The slurry is incubated for five minutes, and then re-suspended using a transfer pipette. This process is repeated 3×, and then gravity flow resumed and the elution fraction is collected.

One mg of bovine albumin is added to the his bind elution fraction, using a 7.5% stock solution (Gibco). The elution fraction is then concentrated to 1 mL using 5 kD MWCO Ultracel centrifugal concentrator (Millipore). The concentrate is then dialyzed into 50 mM NaPO4, 109 mM NaCl pH 7.3 via 3×1:500 exchanges using a Medi Tube-O-Dialyzer (GenoTech). The dialyzed product is 0.22 um filtered using a Spin-X centrifuge tube filter (Costar/Corning), aliquotted, and stored at −80° C.

### Example 10

In Vivo Models for Measuring Effects of Zsig98 on Metabolism

In one in vivo model each member of a group (containing six female, one year old BALB/c mice) is injected with cells transfected with an expression vector containing the Zsig98 gene. Blood is drawn at days 12 and 15 (non-fasted), and at day 19 (fasted). Serum glucose levels (days 12, 15 and 19) and serum insulin levels (days 12 and 15) are determined, as well as cell counts, complete blood chemistries and complete blood counts (CBCs).

In a second in vivo model female BALB/c mice (female, 9 weeks old) are intraperitoneally injected at day 0 with cells transfected with an expression vector containing the Zsig98 gene. All of the animals are fasted prior to being bled on days −3, 8, 12 and 27. For fasting, food is removed at the end of the previous day's light cycle. The animals experience a dark cycle without food, and then the animals are bled after the beginning of the next light cycle. Thereafter, food is restored. Serum glucose is measured using serum obtained from whole blood collected in non-heparinized tubes. The blood is centrifuged immediately and the serum is analyzed for glucose concentration. Serum triglyceride levels are alsom measured

In a third in vivo model 8 month old db/db mice (very obese, severely diabetic) are injected with cells transfected with an expression vector containing the Zsig98 gene. Non-fasted animals are bled on days −4, 7, 13 and 17 and blood urea nitrogen levels (an indicator of kidney function) are measured.

From the mice in the models described above, the pancreas and spleen, a portion of the small intestine, omentum and any omental fat that might include pancreas are collected. The tissues are fixed in 10% NBF (neutral buffered formalin; Surgipath, Richmond, Ill.) overnight. The pancreatic lobes are pressed together slightly to expose the largest pancreatic area to make every lobe of the pancreas flatten. The tissue is dehydrated with a graded series of ethyl alcohols, cleared with xylene, and infiltrated with Paraplast X-tra (Fisher Scientific, Pittsburgh, Pa.) using a Tissue-Tek VIP2000 (Miles, Inc., Elkhart, Ill.). The flattened pancreas is removed from the biopsy bag using forceps and embedded longitudinally with Paraplast X-tra. All pancreata are oriented the same way in the block, with the head of the pancreas placed in one corner of the embedding mold, the tail of the pancreas in the opposite corner, and the body in the middle of the mold. Each section is trimmed with a Jung Biocut 2035 microtome (Bartels and Stout, Inc., Bellevue, Wash.) until the largest pancreatic profile area is exposed. Sections are cut at 3 um in thickness.

The sections are stained with Harris hematoxylin (Sigma, St. Louis, Mo.) and Eosin histology staining (Surgipath, Richmond, Ill.). The number and size of islets per longitudinal section of the pancreas are counted and measured by using a camara-lucida attached to a light microscope (10× objective, Olympus, BH-2), interfaced to a BioQuant System IV image analysis system (B&M Biometric, Inc., Nashville, Tenn.). After calibration, the electronic pen of the digitizer is used to carefully trace the outline of each islet profile by screening the whole section of the pancreas. Simultaneously, the data is computed and stored. Data analyses are performed by using ANOVA (GraphPad Software, San Diego, Calif.) followed by unpaired t test. Increases in the number of islets present, as well as the size of the islets, in samples taken from animals cells transfected with an expression vector containing the Zsig98 gene compared to the control animals is measured.

In another model, purified Zsig98 that is produced by co-expressing the protein with PC3, is administered to normal mice to evaluate the effects on blood glucose and pancreatic islet histomorphometry. The duration of the study is 27 days with dosing for 20 days. Female Balb/c mice, approximately nine weeks old are divided into the following treatment groups as follows: Group 1: Vehicle (0.1% BSA/PBS), ip, n=10; Group 2: 1 μg zins1/PC3 per mouse (50 g/kg), ip, n=10; Group 3: 5 μg zins1/PC3 per mouse (250 μg/kg), ip, n=10; and Group 4: Untreated, n=10

On day 0, mice are weighed, ear tagged and injected with 0.1 ml of the appropriate treatment solution. Animals are checked daily for behavioral and grooming changes, and body weights are determined weekly.

Labeling with BrdU (Zymed Laboratories, South San Francisco, Calif.), according the manufacturer's specifications is done from days 8-11 and from days 17-19 to label islet cells that are dividing in response to zins1.

Animals are bled on day 8 (a non-fasting sample) under ether anesthesia for clinical chemistry.

Mice are weighed and bled for serum on day 28. At necropsy, on day 28, the pancreas and a piece of gut for BrdU control are collected. The pancreas is processed for histomorphometric analysis of islet size and number as described in A.ii., above. In addition, total cells and islets are analyzed for BrdU incorporation as described in Ellwart et al., Cytometry 6:513 -520, 1985.

### Example 11

In Vitro Assays for Measuring Effects of Zsig98 on Metabolism

A. Isolation of Positive Control for Islet Proliferation Assay

To establish an assay to measure proliferation in islets in vitro, a positive control is isolated and characterized as fetal antigen 1 (FA1) as follows:

Pancreata from four 8-11 week old, p53 −/− male mice (Taconic Farms, Germantown, N.Y.) are excised. The dissected pancreata are placed in a sterile 30 mm petri dish containing 7 ml of HBSS (Table 5)+5 mM CaCl2, and the tissue is minced for exactly 2 minutes. Using a 10 ml pipet, the tissue is transferred to a sterile 25 ml screw-capped, round-bottom centrifuge tube, and 20 ml HBSS+5 mM CaCl2. was added. After settling (about 2 minutes), the supernatant (containing fat and connective tissue) is removed. This procedure is repeated twice.

24 mg collagenase (Collagenase Type XI, Sigma Chemical Co., St. Louis, Mo.) is dissolved in 12 ml HBSS+5 mM CaCl₂ just prior to use, and kept on ice. The collagenase solution (6 ml) is added to the minced tissue to a final concentration of 2 mg/ml. The cell mixture is placed on a shaker (300 rpm at. 37° C.) for 15 minutes, and then quickly centrifuged for ˜2 minute at 800 rpm in a Beckman CS-6R centrifuge with a swinging bucket rotor (Beckman Instruments, Palo Alto, Calif.). The supernatant is discarded.

Six ml fresh collagenase solution and 800 *l DNAse are added, and the cell mixture returned to the shaker for up to 20 minutes. 50 *l of cell mixture sample is added to 150 *l DTZ, and examined using a dissecting microscope to ascertain when the islet cells are isolated, but not over-digested.

When the islet cells are isolated, the collagenase digestion is stopped by adding 15 ml HBSS+10% FBS to the mixture, and the mixture is then centrifuged in a Beckman CS-6R centrifuge with a swinging bucket rotor (Beckman Instruments, Palo Alto, Calif.) ˜2 minutes at 800 rpm (the “wash step”). The supernatant is removed and discarded. The wash step is repeated two more times.

After washing, the cell pellet is resuspended in 2 ml HBSS, and the resuspended preparation is placed on two PERCOLL gradients (3 ml 40% PERCOLL and 3 ml 60% PERCOLL per 50 ml tube). One ml of this cell suspension is added to each tube. An additional 2 ml of HBSS is used to sequentially rinse the tubes from which the cell pellets are previously removed. This 2 ml of rinse suspension is added in 1 ml aliquots to each of the two gradients. Thus, each 50 ml tube has 2 ml of cell suspension on the top, then 3 ml of 40% PERCOLL, and finally 3 ml of 60% PERCOLL. The tubes are centrifuged in a Beckman CS-6R centrifuge with a swinging bucket rotor (Beckman Instruments) at 1850 rpm for 20 minutes, without the brake on.

After centrifugation, the top and bottom gradient interfaces are removed with a sterile transfer pipet, and each interface is transferred to a separate 50 ml tube. HBSS+10% FBS is added to the interface and washed by centrifugation in a Beckman CS-6R centrifuge with a swinging bucket rotor (Beckman Instruments) for 10 minutes at 925 rpm.

The top and bottom interfaces are filtered through a 70 *m nylon cell strainer (Becton Dickinson & Co., San Jose, Calif.). The islet cells remain on the filter, and exocrine tissue passes through. The filter is flipped upside-down in a 60 mm petri dish, and the islet cells are washed into the dish. To ensure their isolation from other tissue, the islet cells are plucked into a clean 60 mm non-tissue culture-treated dish containing RPMI growth medium (Table 8)+10% FBS. The islets are incubated at 37° C., 5% CO2 and the medium is changed at 24 and 48 hours.

Islets, obtained as described above, are placed in a 60 mm petri dish in RPMI+10% FBS, and nine days later the whole islets are removed from the petri dish and replated in another 60 mm petri dish. Twenty one days later, the first dish is confluent, and the cells are removed with trypsin and passed into a T25 flask.

Conditioned culture medium removed from these islet cells is added to cultures of normal BALB/c islets are isolated in Matrigel Basement Membrane Matrix (Collaborative Biomedical Products, Bedford, Mass.). Mouse islet phenotype is observed for changes

A BrdU incorporation study using BALB/c islets incubated with IDC53.1 conditioned medium (CM) is performed, to test whether there are cells within the islets that were proliferating. Briefly, one group of four T12.5 flasks (Becton Dickinson) is inoculated with 100 islets each, and 5×IDC53.1 CM+0.5% FBS is added to each flask. Another group of three T12.5 flasks is inoculated with 100 islets each, and SFEF medium (serum free/insulin free medium; Becton Dickinson)+0.5% FBS is added.

BrdU (Becton Dickinson) is added to the islet cell cultures daily, to a final concentration of 10 □M. A flask from each group is harvested on days 4, 8 and 12. On day 8, two of the four flasks in the IDC53.1 CM test group are harvested. One of these flasks is used for an isotype control. The protocol and reagents for BrdU assay are available from Becton Dickinson Immunocytometry Systems, San Jose, Calif., and are used according to the manufacturer's specifications.

For each harvested flask, the islets are harvested, washed twice in 1% BSA/PBS, and centrifuged at 800 rpm for 10 minutes. The pellet is resuspended in 200 *l of 1×PBS on ice. Islets are slowly added to 2.5 ml cold 70% ethanol in a siliconized glass tube while maintaining a vortex. The islets are incubated on ice for 30 minutes, and the result is fixed islet cells. The fixed islets are centrifuged at 1000 rpm for 10 minutes at 10° C., and the ethanol is carefully removed.

One ml of 2 N HCl/Triton X-100 is slowly added to the cells, a few drops at a time, while maintaining a vortex. The mixture is incubated at room temperature for 30 minutes, to denature the DNA and produce single-stranded molecules. The preparation is centrifuged at 1000 rpm for 10 minutes, and then the supernatant is removed and the pellet resuspended in 1 ml of 0.1 M Na2B407.10H20, pH 8.5, to neutralize the acid. The resultant cells may be stored at this point by centrifuging, resuspending in cold 70% ethanol, and storing at −20° C.

The cells are then centrifuged at 1000 rpm for 10 minutes, washed with 1 ml of 0.5% TWEEN 20 in 1% BSA/PBS (TWEEN/BSA/PBS), and resuspended in 100 □l TWEEN/BSA/PBS. To this resuspended preparation is added 20 □l of FITC-labeled anti-BrdU antibody or isotype control. The mixture is incubated overnight on a shaker at 4° C. for whole islets. Thereafter, the cells are washed 3 times using 1 ml TWEEN/BSA/PBS, where each wash is performed for at least 2 hours on the shaker. Preferable, the final wash is left overnight.

The islet preparation is then mounted on glass slides with depressions to prevent the islets from losing their shape. FluoroGuard Antifade Reagent (BioRad, Hercules, Calif.) is the mounting medium used. All positive BrdU cells per islet are counted for each of the three harvest days. Islets are prepared as described above for a BrdU assay; but after incubation with the BrdU, the islets are harvested, fixed, embedded, sliced and stained for anti-Brdu, anti-insulin, anti-glucagon and anti-somatastatin using standard immunohistochemistry techniques.

B. Zsig98 Testing in In Vitro Islet Assay

Normal BALB/c islets are isolated from 8.5 week old male mice. The islets are plated into a 96-well flat bottom plate, with approximately 15 islets/well in serum-free/insulin-free+0.5% FCS medium, in duplicate. Zsig98 diluted serum-free/insulin-free+0.5% FCS medium is added at concentrations of 1-20 ng/ml, along with a negative control of serum-free/insulin-free+0.5% FCS medium, and a positive control of conditioned medium as described in A, above.

The ability of Zsig98 to maintain islets in a viable condition as well as to further stimulate expansion of specific cell types by outgrowth from the islets is measured.

### Example 12

Serum Titer from Rabbit Anti-Human Zsig98

Conjugation of synthetic zsig98 to cBSA for Polyclonal antibody production was performed as follows. Conjugation was performed with the Pharmalink Kit (Pierce, prod number 77158): 2.2 mg zsig98 peptide (dry, PEP05014, MW. 6698, PI 7.08) was solubilized in 0.5 ml water. 0.2 ml Pierce Pharmalink Conjugation buffer (0.1M MES, 0.15M NaCl, pH 4.7; product no. 77167, Lot No. FG72995) was added and the concentrated antigen was transferred to a fresh tube. 2 mg cationic BSA (Pierce Immject, No. 77168) (˜15 nmol) was added and dissolved in 150 ul coupling buffer and mix well. 50 ul of coupling reagent (Pierce No. 77168, Lot no. FL93752) (37% formaldehyde) was added to the carrier/hapten solution to a final concentration=˜3.6%. The solution was mixed well and rocked overnight at 37° C. The next day, the conjugate was transferred to an Ultrafree0-4 cartridge (5000 MWCO) and concentrated to <0.5 ml. Greater than 2 ml of PBS was added and concentrate again. The concentrated antigen was resuspended in PBS to the desired concentration. ˜4 mg total protein/1.5 ml, 2.6 mg/ml; ˜50% zsig98. The antigen was used to immunize rabbits and sequential bleeds were taken for titer determinations. One rabbit had a good titer response.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. An isolated polypeptide consisting of the amino acid sequence as shown in SEQ ID NO: 18 from amino acid 1 to amino acid 61.

2. A composition for improving nutrient absorption wherein the composition comprises the polypeptide according to claim 1 in combination with a nutrient mixture.

3. The composition according to claim 2 wherein the nutrient mixture is milk or a milk substitute.

4. A method of modulating secretion of hormones in vitro or in vivo comprising administering the polypeptide according to claim 1, whereby the polypeptide forms a peptide-receptor complex, and wherein the formation of the peptide-receptor complex modulates the secretion of hormones in the cells.

5. The method according to claim 4, wherein the hormone is insulin or glucagon.

6. A method of inducing secretion of enzymes and hormones secreted from endocrine, exocrine, or gastrointestinal cells comprising administering the polypeptide consisting of residues 1 to 61 of SEQ ID NO: 18, whereby the administration results in secretion of enzymes and hormones.

7. The method according to claim 6, wherein the hormone secreted is glucagon or insulin.

8. The method according to claim 6, wherein the cells are alpha, beta, or acinar cells.

9. A method of treating a mammal having a having a metabolic disorder, wherein the metabolic disorder is selected from the group consisting of:
a) satiety regulation;
b) glucose absorption;
c) glucose metabolism; and
d) neuropathy-associated gastrointestinal disorders
and wherein the method comprises, administering the polypeptide consisting of residues 1 to 61 of SEQ ID NO: 18, and whereby the metabolic state of mammal having the metabolic disorder is improved.

10. A method of stimulating glucose-induced insulin release in a mammal comprising administering the polypeptide consisting of residues 1 to 61 of SEQ ID NO: 18, whereby the administration induces insulin release in the mammal.

11. An isolated polypeptide consisting of the amino acid sequence as shown in SEQ ID NO: 23.

12. A method of inducing proliferation of cells in the pancreas comprising exposing the cells to a polypeptide consisting of residues 1 to 61 of SEQ ID NO: 18, whereby the exposure of the cells to the polypeptide causes the pancreas cells to increase in number

13. The method according to claim 12, wherein the cells are endocrine cells.

14. The method according to claim 13 wherein the cells are selected from:
a) acinar cells;
b) alpha cells; and
c) beta cells.

15. A method of increasing pancreatic cell mass comprising exposing the pancreas cells to a polypeptide consisting of residues 1 to 61 of SEQ ID NO: 18, whereby the exposure of the cells to the polypeptide causes the pancreatic cell mass to increase.

16. The method according to claim 15, wherein the cells are endocrine cells

17. The method according to claim 16, wherein the cells are selected from:
a) acinar cells;
b) alpha cells; and
c) beta cells.

18. A method of producing multimers of the polypeptide consisting of residues 1 to 61 of SEQ ID NO: 18, comprising:
a) introducing the polynucleotide encoding the polypeptide consisting of residues 1 to 61 if SEQ ID NO: 18 into an expression vector;
b) transfecting a host cell with the expression vector whereby the host cell expresses the polypeptide encoded by the polynucleotide; and
c) recovering the polypeptide.

19. The method according the claim 18, wherein the host cell is eukaryotic.

20. The method according to claim 19, wherein the host cell is prokaryotic,

21. The method according to claim 18, wherein the multimers are dimers.

22. A method of producing multimers of the polypeptide consisting of residues 1 to 61 of SEQ ID NO: 18, comprising producing the multimers by chemical synthesis.

23. The method according to claim 22, wherein the multimers are dimers.

24. An isolated polypeptide complex comprising multiple copies of the polypeptide consisting of residues 1 to 61 of SEQ ID NO: 18.

25. The isolated polypeptide complex according to claim 24, wherein the number of copies is two.

26. The isolated polypeptide complex according to claim 25, wherein the complex is formed by disulfide bonds.

27. An isolated antibody, wherein the antibody specifically bind to monomers, dimers, or multimers of the Zsig98 polypeptides.
